(19) United States
(12) Patent Application Publication
(10) Pub. No.: US 2010/0016889 A1
(43) Pub. Date: Jan. 21, 2010

(54) METHODS AND APPARATUS FOR ANULUS REPAIR

(75) Inventor: Bret A. Ferree, Cincinnati, OH (US)

Correspondence Address: GIFFORD, KRASS, SPRINKLE,ANDERSON & CITKOWSKI, P.C, PO BOX 7021, TROY, MI, 48007-7021, US

(73) Assignee: Anova Corporation, Summit, NJ (US)

(21) Appl. No.: 12/263,753

(22) Filed: Nov. 3, 2008

Related U.S. Application Data
(63) Continuation-in-part of application No. 11/811,751, filed on Jun. 12, 2007.
(60) Provisional application No. 60/813,232, filed on Jun. 13, 2006.
(60) Provisional application No. 60/847,649, filed on Sep. 26, 2006.
(60) Provisional application No. 60/984,657, filed on Nov. 1, 2007.

(51) Int. Cl. A61B 17/04

(52) U.S. Cl. 606/228

(57) ABSTRACT

Apparatus and methods facilitates reconstruction of the anulus fibrosus (AF) and/or the nucleus pulposus (NP) to prevent recurrent herniation following microlumbar discectomy. The invention may also be used in the treatment of herniated discs, anular tears of the disc, or disc degeneration, while enabling surgeons to preserve the contained nucleus pulposus. A spinal repair system according to the invention comprises flexible longitudinal fixation components adapted for placement through portions of the AF with intact fibers, a porous mesh reinforcement component adapted for placement over a region of the AF with damaged fibers, and an anti-adhesion component for placement over flexible longitudinal fixation components and the porous mesh component. Preferred embodiments of the invention include an intra-aperture component dimensioned for positioning within a defect in the AF, with one or more components being used to maintain the intra-aperture component in position. One or more lengthwise passageways through the intra-aperture component, one or more lengthwise grooves on the outer surface of the intra-aperture component, or a combination thereof, intentionally facilitate the escape of nucleus pulposus tissue through or around the intra-aperture component in response to pressure applied by the upper and lower vertebral bodies.

### REFERENCE TO RELATED APPLICATIONS

This application claims priority from U.S. Provisional Patent Application Ser. No. 60/984,657, filed Nov. 1, 2007. This application is also a continuation-in-part of U.S. patent application Ser. No. 11/811,751, filed Jun. 12, 2007, which claims priority from U.S. Provisional Patent Application Ser. Nos. 60/813,232, filed Jun. 13, 2006 and 60/847,649, filed Sep. 26, 2006. The entire content of each application is incorporated herein by reference.

### FIELD OF THE INVENTION

This invention relates generally to the treatment of intervertebral disc herniation and degenerative disc disease and, in particular, to apparatus and methods for fortifying and/or replacing disc components such as the anulus fibrosis.

### BACKGROUND OF THE INVENTION

The human intervertebral disc is an oval to kidney bean-shaped structure of variable size depending on the location in the spine. The outer portion of the disc is known as the anulus fibrosus (AF, also known as the “anulus fibrosis”). The anulus fibrosus (AF) is made of ten to twenty collagen fiber lamellae. The collagen fibers within a lamella are parallel. Successive lamellae are oriented in alternating directions. About 48 percent of the lamellae are incomplete, but this value varies based upon location and increases with age. On average, the lamellae lie at an angle of sixty degrees with respect to the vertebral axis line, but this too varies depending upon location. The orientation serves to control vertebral motion (one half of the bands tighten to check motion when the vertebra above or below the disc are turned in either direction).

The anulus fibrosus contains the nucleus pulposus (NP). The nucleus pulposus serves to transmit and dampen axial loads. A high water content (approximately 70-80 percent) assists the nucleus in this function. The water content has a diurnal variation. The nucleus imbibes water while a person lies recumbent. Nuclear material removed from the body and placed into water will imbibe water swelling to several times its normal size. Activity squeezes fluid from the disc. The nucleus comprises roughly 50 percent of the entire disc. The nucleus contains cells (chondrocytes and fibrocytes) and proteoglycans (chondroitin sulfate and keratin sulfate). The cell density in the nucleus is on the order of 4,000 cells per microliter.

The intervertebral disc changes or “degenerates” with age. As a person ages, the water content of the disc falls from approximately 85 percent at birth to approximately 70 percent in the elderly. The ratio of chondroitin sulfate to keratin sulfate decreases with age, while the ratio of chondroitin 6 sulfate to chondroitin 4 sulfate increases with age. The distinction between the anulus and the nucleus decreases with age. Generally disc degeneration is painless.

Premature or accelerated disc degeneration is known as degenerative disc disease. A large portion of patients suffering from chronic low back pain are thought to have this condition. As the disc degenerates, the nucleus and anulus functions are compromised. The nucleus becomes thinner and less able to handle compression loads. The anulus fibers become redundant as the nucleus shrinks. The redundant anular fibers are less effective in controlling vertebral motion. This disc pathology can result in: I) bulging of the anulus into the spinal cord or nerves; 2) narrowing of the space between the vertebra where the nerves exit; 3) tears of the anulus as abnormal loads are transmitted to the anulus and the anulus is subjected to excessive motion between vertebra; and 4) disc herniation or extrusion of the nucleus through complete anular tears.

Current surgical treatments for disc degeneration are destructive. One group of procedures, which includes lumbar discectomy, removes the nucleus or a portion of the nucleus. A second group of procedures destroy nuclear material. This group includes Chymopapin (an enzyme) injection, laser discectomy, and thermal therapy (heat treatment to denature proteins). The first two groups of procedures compromise the treated disc. A third group, which includes spinal fission procedures, either removes the disc or the disc's function by connecting two or more vertebra together with bone. Fusion procedures transmit additional stress to the adjacent discs, which results in premature disc degeneration of the adjacent discs. These destructive procedures lead to acceleration of disc degeneration.

Prosthetic disc replacement offers many advantages. The prosthetic disc attempts to eliminate a patients pain while preserving the disc's function. Current prosthetic disc implants either replace the nucleus or replace both the nucleus and the anulus. Both types of current procedures remove the degenerated disc component to allow room for the prosthetic component. Although the use of resilient materials has been proposed, the need remains for further improvements in the way in which prosthetic components are incorporated into the disc space to ensure strength and longevity. Such improvements are necessary, since the prosthesis may be subjected to 100,000,000 compression cycles over the life of the implant.

Current nucleus replacements (NRs) may cause lower back pain if too much pressure is applied to the anulus fibrosus. As discussed in co-pending U.S. Pat. Nos. 6,878,167 and 7,201,774. the content of each being expressly incorporated herein by reference in their entirety, the posterior portion of the anulus fibrosus has abundant pain fibers.

Herniated nucleus pulposus (HNP) occurs from tears in the anulus fibrosus. The herniated nucleus pulposus often allies pressure on the nerves or spinal cord. Compressed nerves cause back and leg or arm pain. Although a patient's symptoms result primarily from pressure by the nucleus pulposus, the primary pathology lies in the anulus fibrosus.

Surgery for herniated nucleus pulposus, known as microlumbar discectomy (MLD), only addresses the nucleus pulposus. The opening in the anulus fibrosus is enlarged during surgery, further weakening the anulus fibrosus. Surgeons also remove generous amounts of the nucleus pulposus to reduce the risk of extruding additional pieces of nucleus pulposus through the defect in the anulus fibrosus. Although microlumbar discectomy decreases or eliminates a patient's leg or arm pain, the procedure damages weakened discs.

### SUMMARY OF THE INVENTION

The invention broadly facilitates reconstruction of the anulus fibrosus (AF) and the nucleus pulposus (NP). Such reconstruction prevents recurrent herniation following microlumbar discectomy. The invention may also be used in the treatment of herniated discs, anular tears of the disc, or disc degeneration, while enabling surgeons to preserve the contained nucleus pulposus. The methods and apparatus may be used to treat discs throughout the spine including the cervical, thoracic, and lumbar spines of humans and animals.

The invention also enables surgeons to reconstruct the anulus fibrosus and replace or augment the nucleus pulposus. Novel nucleus replacements (NR) may be added to the disc. Anulus reconstruction prevents extrusion of the nucleus replacements through holes in the anulus fibrosus. The nucleus replacements and the anulus fibrosus reconstruction prevent excessive pressure on the anulus fibrosus that may cause back or leg pain. The nucleus replacements may be made of natural or synthetic materials. Synthetic nucleus replacements may be made of, but are not limited to, polymers including polyurethane, silicon, hydrogel, or other elastomers.

A spinal repair system according to the invention comprises flexible longitudinal fixation components adapted for placement through portions of the AF with intact fibers, a porous mesh reinforcement component adapted for placement over a region of the AF with damaged fibers, and an anti-adhesion component for placement over flexible longitudinal fixation components and the porous mesh component. The invention also includes a targeting device that may be used to determine injured and uninjured areas of the AF that lie adjacent to a fissure or aperture in the AF.

Preferred embodiments of the invention include an intra-aperture component dimensioned for positioning within a defect in the AF, with one or more components being used to maintain the intra-aperture component in position. One or more lengthwise passageways through the intra-aperture component, one or more lengthwise grooves on the outer surface of the intra-aperture component, or a combination thereof, intentionally facilitate the escape of nucleus pulposus tissue through or around the intra-aperture component in response to pressure applied by the upper and lower vertebral bodies.

The intra-aperture component may be porous and flexible while being intentionally non-expandable in cross section following its positioning within the defect. A component used to maintain the intra-aperture component within the defect includes a flexible longitudinal fixation component that passes through the intra-aperture component and a region of the AF apart from the defect. If available, this may be a region of the AF having overlapping layers with intact fibers in different directions.

The flexible longitudinal fixation component may pass through a generally vertical passageway in the intra-aperture component and a region of the AF apart from the defect. The flexible longitudinal fixation component may anchored to one of the upper and lower vertebral bodies. The components used to maintain the intra-aperture component within the defect includes a flexible longitudinal fixation component that passes twice through the intra-aperture component and is anchored to one of the upper and lower vertebral bodies. For example, the flexible longitudinal fixation component may form one or more loop or loops, each passing once through the AF and twice through the intra-aperture component.

### BRIEF DESCRIPTION OF THE DRAWINGS

FIGS. 1A and 1B show posterior views of a coronal cross section of a portion of the spine;

FIG. 1C is an illustration from a textbook that shows the architecture of the anulus fibrosis;

FIG. 1D is a posterior view of an intervertebral disc (IVD);

FIG. 1E is a posterior view of the IVD shown in FIG. 1D and horizontal and vertical suture bands that surround the overlapping portions of the AF fibers;

FIGS. 2A-2F are posterior views of a coronal cross section of a portion of the spine;

FIGS. 3A and 3B show a posterior views of an intervertebral disc;

FIG. 3C is a posterior view of the AF and an alternative embodiment of the invention shown in FIG. 3B;

FIG. 4A is a posterior view of the AF and the zones of injury;

FIG. 4B is a view of the top of an alternative embodiment of the invention;

FIG. 4C is a posterior view of an IVD and the embodiment of the invention drawn in FIG. 4B;

FIG. 4D is a drawing of a surgeon's view through the oculars of an operating microscope and the embodiment of the invention drawn in FIG. 4C;

FIG. 4E is a drawing of a surgeon's view through the oculars of an operating microscope and the embodiment of the invention drawn in FIG. 4D;

FIG. 4F is a drawing of a surgeon's view through the oculars of an operating microscope and the embodiment of the invention drawn in FIG. 4E;

FIG. 4G is a view through the oculars of an operating microscope and a posterior view of the embodiment of the invention drawn in FIG. 4D;

FIG. 5A is a posterior view of the AF;

FIG. 5B is a posterior view of an alternative embodiment of the invention;

FIG. 5C is a posterior view of an IVD and the embodiments of the invention shown in FIGS. 5A and 5B;

FIG. 5D is a posterior view of an IVD and the embodiment of the invention shown in FIG. 5C;

FIG. 5E is a posterior view of an IVD and the embodiment of the invention drawn in FIG. 5D;

FIG. 6A is a posterior view of an IVD, a caudal cross section of a vertebra and an alternative embodiment of the invention drawn in FIG. 5A;

FIG. 6B is a posterior view of an IVD, coronal cross sections of two vertebrae, and the embodiment of the invention drawn in FIG. 6A;

FIG. 7A is a posterior view of an alternative embodiment of the invention drawn in FIG. 5B;

FIG. 7B is a posterior view of a coronal cross section of a portion of the spine and the embodiment of the invention drawn in FIGS. 6B and 7A;

FIG. 8 is a posterior view of a coronal cross section of a portion of the spine, the embodiment of the invention drawn in FIG. 7B and an alternative embodiment of the invention drawn in FIG. 5E;

FIG. 9A is a posterior view of an IVD and an alternative embodiment of the invention drawn in FIG. 5A;

FIG. 9B is an axial cross section of an IVD and the embodiment of the invention drawn in FIG. 9A;

FIG. 9C is a posterior view of an IVD and the embodiment of the invention drawn in FIG. 9A;

FIG. 9D is an axial cross section of an IVD and the embodiment of invention drawn in FIG. 9C;

FIG. 9E is a posterior view of an IVD and the embodiment of the invention drawn in FIG. 9C;

FIG. 9F is a posterior view of an IVD and the embodiments of the invention drawn in FIGS. 5D and 9A-E;

FIG. 9G is a posterior view of an IVD, the embodiment of the invention drawn in FIG. 9F and an alternative embodiment of the invention drawn in FIG. 5E;

FIG. 9H is a posterior view of an IVD and the embodiment of the invention drawn in FIG. 9G;

FIG. 10A is a posterior view of an alternative embodiment of the invention;

FIG. 10B is an end view of the embodiment of the invention drawn in FIG. 10A;

FIG. 10C is a posterior view of the embodiment of the invention drawn in FIG. 10A;

FIG. 10D is a posterior view of an IVD and the embodiment of the invention drawn in FIG. 10C;

FIG. 10E is a posterior view of an IVD and the embodiment of the invention drawn in FIG. 10D;

FIG. 10F is a posterior view of an IVD and the embodiment of the invention drawn in FIG. 10E;

FIG. 10G is an axial cross section of an IVD and the embodiment of the invention drawn in FIG. 10F;

FIG. 10H is a posterior view of an IVD and the embodiment of the invention drawn in FIG. 10G;

FIG. 10I is a posterior view of an IVD and the embodiment of the invention drawn in FIG. 10H;

FIG. 11A is a lateral view of an anchor or fixation member and a portion of a flexible longitudinal fixation element:

FIG. 11B is a lateral view of the embodiment of the invention drawn in FIG. 11A;

FIG. 11C is a longitudinal cross section of the embodiment of the invention drawn in FIG. 11B;

FIG. 11D is an exploded lateral view of the embodiment of the invention drawn in FIG. 11C;

FIG. 11E is a view of the distal, cone, end of the embodiment of the invention drawn in FIG. 11C;

FIG. 11F is a view of the proximal end of the embodiment of the invention drawn in FIG. 11C;

FIG. 11G is a view of the distal end of the embodiment of the invention drawn in FIG. 11C;

FIG. 11H is a view of the proximal end of the embodiment of the invention drawn in FIG. 11C;

FIG. 11I is a lateral view of an alternative embodiment of the invention drawn in FIG. 11B;

FIG. 11J is a lateral view of an alternative embodiment of the invention drawn in FIG. 11I;

FIG. 11K is a partial sagittal cross section of a portion of the spine and the embodiment of the invention drawn in FIG. 11J;

FIG. 11L is a lateral view of an alternative embodiment of the invention drawn in FIG. 11A;

FIG. 11M is a lateral view of the embodiment of the invention drawn in FIG. 11L;

FIG. 12A is a view of the proximal end of an alternative embodiment of the invention drawn in FIG. 11H;

FIG. 12B is a posterior view of an IVD;

FIG. 12C is a view of the inner portion of the posterior AF;

FIG. 12D is a view of the inner portion of the posterior AF;

FIG. 12E is a partial sagittal cross section of a portion of the spine and the embodiment of the invention drawn in FIG. 12C;

FIG. 12F is a partial sagittal cross section of a portion of the spine and the embodiment of the invention drawn in FIG. 12E;

FIG. 13A is a view of the proximal end of an alternative embodiment of the invention drawn in FIG. 11A;

FIG. 13B is a posterior view of the inner portion of the AF and the embodiment of the invention drawn in FIG. 11G;

FIG. 14A is a lateral view of the embodiment of the invention drawn in FIG. 11A and a tool used to insert the device into the spine;

FIG. 14B is a longitudinal cross section of the embodiment of the invention drawn in FIG. 14A;

FIG. 14C is an exploded longitudinal cross section of the embodiment of the invention drawn in FIG. 14B;

FIG. 14D is a lateral view of an alternative embodiment of the invention drawn in FIG. 14A;

FIG. 14E is a longitudinal cross section of the embodiment of the invention drawn in FIG. 14D;

FIG. 14F is a view of the top of the spaced component drawn in FIG. 14E;

FIG. 15A is an axial cross section of an IVD and the embodiment of the invention drawn in FIG. 14A;

FIG. 15B is an exploded axial cross section of an IVD and the embodiment of the invention drawn in FIG. 15A;

FIG. 15C is an axial cross section of an IVD and the embodiment of the invention drawn in FIG. 15B;

FIG. 15D is an axial cross section of an IVD, and the embodiments of the invention drawn in FIGS. 5B and 15C;

FIG. 15E is an axial cross section of an IVD and the embodiment of the invention drawn in FIG. 15D;

FIG. 15F is a posterior view of an IVD and the embodiment of the invention drawn in FIG. 15E;

FIG. 16A is a lateral view of an alternative embodiment of the invention;

FIG. 16B is a lateral view of the embodiment of the invention drawn in FIG. 16A;

FIG. 16C is an axial cross section of an IVD and the embodiment of the invention drawn in FIG. 16A;

FIG. 16D is an axial cross section of an IVD and the embodiment of the invention drawn in FIG. 16B;

FIG. 17A is a lateral view of the embodiment of the invention drawn in FIG. 16A, an anti-adhesion cover, and a tool used to insert the embodiment of the invention drawn in FIG. 16A;

FIG. 17B is a lateral view of a portion of the spine and the embodiment of the invention drawn in FIG. 17A;

FIG. 17C is a lateral view of a portion of the spine and the embodiment of the invention drawn in FIG. 17B;

FIG. 17D is a posterior view of the IVD and the embodiment of the invention drawn in FIG. 17C;

FIG. 17E is a posterior view of the IVD and the embodiment of the invention drawn in FIG. 17D;

FIG. 18 is a posterior view of the IVD and the welded flexible longitudinal fixation elements, and two staple-like devices drawn in FIG. 17E;

FIG. 19 is a posterior view of an IVD and an alternative embodiment of the invention drawn in FIG. 18;

FIG. 20A is a lateral view of an alternative embodiment of the invention drawn in FIG. 11A;

FIG. 20B is a longitudinal cross section of the embodiment of the invention drawn in FIG. 20A;

FIG. 21A is an axial cross section of an IVD, the embodiment of the invention drawn in FIG. 20A, and an alternative embodiment of the invention drawn in FIG. 10H;

FIG. 21B is an axial cross section of an IVD and the embodiment of the invention drawn in FIG. 21A;

FIG. 21C is an axial cross section of an IVD and the embodiment of the invention drawn in FIG. 21B;

FIG. 21D is a posterior view of an IVD and the embodiment of the invention drawn in FIG. 21C;

FIG. 21E is a posterior view of an IVD and the embodiment of the invention drawn in FIG. 21D;

FIG. 22A is a lateral view of an alternative embodiment of the invention drawn in FIG. 20A;

FIG. 22B is a lateral view of the embodiment of the invention drawn in FIG. 22A;

FIG. 23A is a lateral view of an alternative embodiment of the inventions drawn in FIGS. 11A and 22A;

FIG. 23B is a lateral view of the embodiment of the invention drawn in FIG. 23A;

FIG. 24A is a lateral view of alternative embodiments of the inventions drawn in FIGS. 14D and 21A-E;

FIG. 24B is a longitudinal cross section of the insertion tools and a lateral view of the fixation members and composite patch drawn in FIG. 24A;

FIG. 24C is a longitudinal cross section of an alternative embodiment of the invention drawn in FIG. 24B;

FIG. 24D is a posterior view of an IVD and the embodiment of the invention drawn in FIG. 24C;

FIG. 24E is a posterior view of an IVD and the embodiment of the invention drawn in FIG. 24D;

FIG. 24F is a posterior view of the AF and the embodiment of the invention drawn in FIG. 24E;

FIG. 24G is a cross section of the embodiment of the invention drawn in FIG. 24F;

FIG. 25A is a lateral view of an alternative embodiment of the invention drawn in FIG. 24A;

FIG. 25B is a posterior view of an IVD and the embodiment of the invention drawn in FIG. 25A;

FIG. 25C is a posterior view of an IVD and the embodiment of the invention drawn in FIG. 25B;

FIG. 25D is a posterior view of an IVD and the embodiment of the invention drawn in FIG. 25C;

FIG. 26A is an oblique view of an alternative embodiment of the mesh patch and anti-adhesion cover drawn in FIG. 24G;

FIG. 26B is a lateral view of an alternative embodiment of the invention drawn in FIG. 26A;

FIG. 26C is an axial cross section of an IVD and the embodiments of the invention drawn in FIGS. 11J and 26B;

FIG. 27A is a lateral view of a releasable handle;

FIG. 27B is a lateral view of an alternative embodiment of the invention drawn in FIG. 24A;

FIG. 27C is a view of the top of the embodiment of the invention drawn in FIG. 27A;

FIG. 27D is a view of the top of a insertion tool drawn in FIG. 27B;

FIG. 27E is a lateral view of the embodiment of the invention drawn in FIG. 27B;

FIG. 27F is a lateral view of the top of an alternative embodiment of the insertion tools drawn in FIG. 27E;

FIG. 27G is a view of the posterior portion of an IVD and the top of the embodiment of the invention drawn in FIG. 27F;

FIG. 27H is a posterior view of an IVD and the embodiment of the invention drawn in FIG. 27G;

FIG. 27I is a posterior view of the IVD and the embodiment of the invention drawn in FIG. 27H;

FIG. 28 is a posterior view of an IVD and an alternative embodiment of the invention drawn in FIG. 27I;

FIG. 29A is a posterior view of a coronal cross section of a portion of the spine;

FIG. 29B is a lateral view of a partial sagittal cross section of the spinal segment drawn in FIG. 29B;

FIG. 29C is a posterior view of a coronal cross section of a portion of the spine and an alternative embodiment of the invention drawn in FIG. 6B;

FIG. 29D is a lateral view of a partial cross section of the spinal segment and embodiment of the invention drawn in FIG. 29C;

FIG. 29E is an oblique view of the intra-aperture component of the embodiment of the invention drawn in FIG. 29C;

FIG. 29F is an oblique view of a sizing tool that is preferably placed into the aperture in the AF;

FIG. 29G is a lateral view of sagittal cross section of the intra-aperture component drawn in FIG. 29E;

FIG. 29H is a lateral view of a sagittal cross section of the intra-aperture component drawn in FIG. 29E;

FIG. 29I is a posterior view of a coronal cross section of the embodiment of the invention drawn in FIG. 29E;

FIG. 29J is a posterior view of a coronal cross section of the embodiment of the invention drawn in FIG. 29E;

FIG. 29K is an oblique view of the embodiment of the invention drawn in FIG. 29E;

FIG. 29L is an oblique view of an alternative embodiment of the invention drawn in FIG. 29K;

FIG. 29M is a lateral view of a partial sagittal cross section of portion of the spine drawn in FIG. 29B and the first step to insert the embodiment of the invention drawn in FIG. 29K;

FIG. 29N is a posterior view of a partial coronal cross section of the portion of spinal segment and invention drawn in FIG. 29M;

FIG. 29O is a lateral view of a partial sagittal cross section of the portion of the spine drawn in FIG. 29M, the embodiment of the invention drawn in FIG. 29K, and the second step to insert the component into the IVD;

FIG. 29P is a lateral view of a partial sagittal cross section of the portion of the spinal segment drawn in FIG. 29O and the third step in the method to insert the intra-aperture component;

FIG. 29Q is a lateral view of a partial sagittal cross section of the portion of the spinal segment drawn in FIG. 29P and the fourth step in the method to insert the intra-aperture component;

FIG. 29R is a lateral view of a partial sagittal cross section of the portion of the spinal segment drawn in FIG. 29Q, a novel anchor insertion guide, and the fifth step to insert the intra-aperture component;

FIG. 29S is an oblique view of the distal end of the guide drawn in FIG. 29R;

FIG. 29T is a lateral view of a partial sagittal cross section of the portion of the spinal segment drawn in FIG. 29R and the sixth step in the method to insert the intra-aperture component into the IVD;

FIG. 29U is a lateral view of a partial sagittal cross section of the portion of the spinal segment drawn in FIG. 29T and the seventh step in the method to insert the intra-aperture component into the IVD;

FIG. 29V is a lateral view of a partial sagittal cross section of the portion of the spinal segment drawn in FIG. 29U and the final position of the assembled invention drawn in FIG. 29U;

FIG. 29W is a posterior view of a partial coronal cross section of the spinal segment drawn and the embodiment of the invention drawn in FIG. 29V;

FIG. 30A is a posterior view of a partial coronal cross section of a spinal segment and an alternative embodiment of the invention drawn in FIG. 29W;

FIG. 30B is a partial transverse cross section of the IVD and embodiment of the invention drawn in FIG. 30A;

FIG. 31 is a partial transverse cross section of an IVD and an alternative embodiment of the invention drawn in FIG. 30B;

FIG. 32A is a posterior view of a partial coronal cross section through a spinal segment and an alternative embodiment of the invention drawn in FIG. 29W;

FIG. 32B is a lateral view of a partial sagittal cross section of the spinal segment and the embodiment of the invention drawn in FIG. 32A;

FIG. 33 is an oblique view of an alternative embodiment of the invention drawn in FIG. 29E;

FIG. 34A is a lateral view of a partial sagittal cross section of a spinal segment and an alternative embodiment of the invention drawn in 32B;

FIG. 34B is an oblique view of the embodiment of the intra-aperture component drawn in FIG. 34A;

FIG. 35A is a lateral view of a partial sagittal cross section of a spinal segment and an alternative embodiment of the invention drawn in FIG. 34A;

FIG. 35B is an oblique view of the embodiment of the intra-aperture component drawn in FIG. 35A;

FIG. 36A is a transverse cross section of an IVD and an invention that can be used to safely pass sutures or flexible longitudinal fixation elements through the AF;

FIG. 36B is a transverse cross section of the IVD, the embodiment of the invention drawn in FIG. 36B, and the second step to pass a suture through the AF;

FIG. 36C is a transverse cross section of the IVD, the embodiment of the invention drawn in FIG. 36B, and the third step to pass a suture through the AF;

FIG. 36D is a lateral view of a sagittal cross section of the distal portion of the instrument drawn in FIG. 36C;

FIG. 36E is an exploded transverse cross section of the IVD, the embodiment of the invention drawn in FIG. 36C;

FIG. 36F is a view of the top of the insertion tool drawn in FIG. 36E. Similar to the invention drawn in FIG. 29S;

FIG. 36G is a transverse cross section of the IVD, the embodiment of the invention drawn in FIG. 36E;

FIG. 37A is a transverse cross section of the IVD, a suture that was passed through the AF using the embodiment of the invention drawn in FIGS. 36A-G;

FIG. 37B is a transverse cross section of the IVD, the embodiment of the invention drawn in FIG. 37A;

FIG. 37C is a transverse cross section of the IVD, the embodiment of the invention drawn in FIG. 37B, and the third step in the method of passing a suture through the AF;

FIG. 37D is a transverse cross section of the IVD, the embodiment of the invention drawn in FIG. 37C, and the fourth step in the method of passing a suture through the AF;

FIG. 37E is a transverse cross section of the IVD, the embodiment of the invention drawn in FIG. 37D, and the fifth step in the method of passing a suture through the AF;

FIG. 37F is an exploded transverse cross section of the IVD, the embodiment of the invention drawn in FIG. 37E, and the sixth step in the method of passing a suture through the AF;

FIG. 38A is an oblique view of a tube used to create an alternative embodiment of the invention drawn in FIG. 26A;

FIG. 38B is a posterior view of the tube drawn in FIG. 38A;

FIG. 38C is a posterior view of the embodiment of the invention drawn in FIG. 38B;

FIG. 38D is an anterior view of the embodiment of the invention drawn in FIG. 38C;

FIG. 38E is an anterior view of the embodiment of the invention drawn in FIG. 38D;

FIG. 39A is a transverse cross section of the IVD drawn in FIG. 37F and the embodiment of the invention drawn in FIG. 38E;

FIG. 39B is a transverse cross section of the IVD and the embodiment of the invention drawn in FIG. 39A;

FIG. 39C is a posterior view of a coronal cross section of a spinal segment and the embodiment of the invention drawn in FIG. 39B;

FIG. 40A is an oblique view of an alternative embodiment of the tube drawn in FIG. 38A;

FIG. 40B is an oblique view of the embodiment of the invention drawn in FIG. 40A;

FIG. 41A is a lateral view of the distal end of a novel suture;

FIG. 41B is a view of a partial transverse cross section of a portion of an IVD, the foot-plate if an insertion tool, a cannula and the end of the suture drawn in FIG. 41A;

FIG. 41C is a view of a partial transverse cross section of the portion of the IVD and embodiment of the invention drawn in FIG. 41B;

FIG. 41D is a view of transverse cross section of the IVD and embodiment of the invention drawn in FIG. 41C;

FIG. 42 is a lateral view of a partial sagittal cross section of a spinal segment and an alternative embodiment of the invention drawn in FIG. 29D;

FIG. 43 is a lateral view of a partial sagittal cross section of a spinal segment and an alternative embodiment of the invention drawn in FIG. 42;

FIG. 44A is a lateral view of a partial sagittal cross section of a spinal segment and an alternative embodiment of the invention drawn in FIG. 29V;

FIG. 44B is a lateral view of a partial sagittal cross section of a spinal segment and embodiment of the invention drawn in FIG. 44A;

FIG. 44C is a posterior view of a coronal cross section of the spinal segment and embodiment of the invention drawn in FIG. 44B;

FIG. 45A is an anterior view of an allograft or xenograft spinal segment;

FIG. 45B is a transverse cross section of the IVD drawn in FIG. 45A;

FIG. 45C is a lateral view of a sagittal cross section of the inter-aperture invention drawn in FIG. 44C;

FIG. 45D is a view of the top of the embodiment of the intra-aperture invention drawn in FIG. 44C;

FIG. 45E is a view of the bottom of the embodiment of the invention drawn in FIG. 45D;

FIG. 45F is a view of the bottom of an alternative embodiment of the invention drawn in FIG. 45E;

FIG. 46A is a view of a transverse cross section of an IVD and an alternative embodiment of the invention drawn in FIG. 30B;

FIG. 46B is a view of a transverse cross section of the IVD and the embodiment of the invention drawn in FIG. 46A;

FIG. 46C is a view of the top of the embodiment of the intra-aperture invention drawn in FIG. 46A;

FIG. 47 is a transverse cross section of an IVD and an alternative embodiment of the invention drawn in FIG. 46A;

FIG. 48A is a lateral view of a partial sagittal cross section of a spinal segment and an alternative embodiment of the invention drawn in FIG. 44A;

FIG. 48B is a lateral view of a partial sagittal cross section of the spinal segment and the embodiment of the invention drawn in FIG. 48A;

FIG. 48C is a posterior view of a coronal cross section of the spinal segment and the embodiment of the invention drawn in FIG. 48B;

FIG. 48D is a posterior view of a coronal cross section of a spinal segment and an alternative embodiment of the invention drawn in FIG. 48C; and

FIG. 48E is a posterior view of a coronal cross section of a spinal segment and an alternative embodiment of the invention drawn in FIG. 48D.

### DETAILED DESCRIPTION OF THE INVENTION

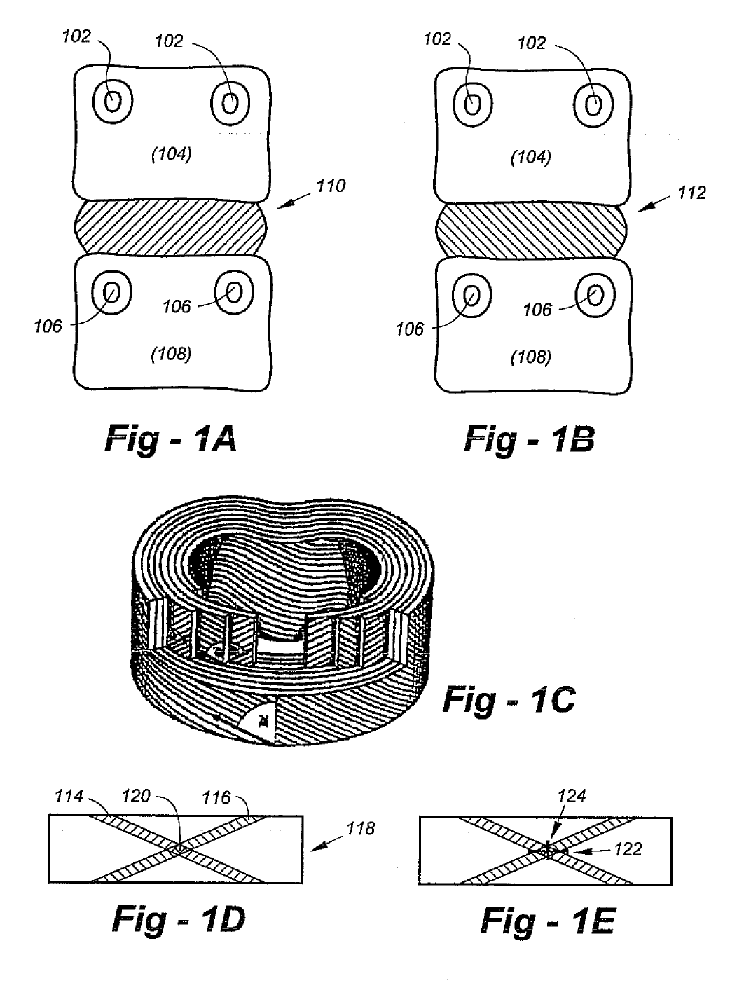

FIG. 1A is a posterior view of a coronal cross section of a portion of the spine. The cross section passes through the pedicles 102, 106 of the vertebrae 104, 108. The fibers of the first layer of the anulus fibrosis (AF) 110 are illustrated at a 60-degree angle relative to the vertical axis of the spine. FIG. 1B is a posterior view of a coronal cross section of a portion of the spine, also passing through the pedicles of the vertebrae. The fibers of the second layer of the AF 112 are illustrated at a 60-degree angle relative to the vertical axis of the spine, but in the opposite direction of the fibers of the adjacent layers of the AF. FIG. 1C is a textbook illustration depicting the structure of the AF, wherein overlapping bands with fibers course in 60-degree angles in opposite directions in successive layers is unique to the intervertebral disc (IVD). The unique structure of the IVD gives the AF properties that are unlike the properties of any other structure in the human or animal body.

FIG. 1D is a posterior view of an IVD. The drawing shows fibers 114, 116 from two adjacent layers of AF 118. Assuming the AF fibers course at a 60-degree angle relative to the vertical axis of the spine, the height of the diamond shaped area of overlap 120 is 58 percent of the width of the overlap. The unique diamond shaped area of overlap provides an opportunity to create unique methods and devices to treat defects in the AF.

FIG. 1E is a posterior view of the IVD drawn in FIG. 1D with horizontal and vertical suture bands 122, 124 that surround the overlapping portions of the AF fibers. The horizontal suture band must be longer than the vertical suture band to surround the overlapping areas of the AF fibers. Based upon the unique structure of the AF and the diamond shaped area of overlap, horizontal suture bands that pass through the AF may grasp 58 percent of the AF fibers that a similar length vertical suture band will grasp. Resistance of sutures to a force that pulls suture bands through the AF may be related to the number of intact AF fibers within the suture bands. The following studies on the IVDs of a human cadaver spine test these theories.

### Examples

A spine (T9-S1) from a 70-year old male donor was bisected in the sagittal plane. The NP was removed from all IVDs. The L3/L4 and L5/S1 levels were severely arthritic and eliminated from further study, thus leaving 7 treatment IVDs. Each IVD underwent the following treatment:

A 5 mm vertical anulotomy (VA) was performed in the anterior lateral portion of the IVD, lateral to the Anterior Longitudinal Ligament (ALL), on the first side of the spine and a 5 mm horizontal anulotomy (HA) was performed in the anterior lateral portion of the IVD on the second side of the spine. A vertical suture was placed in the AF tissue surrounding the HA and a horizontal suture was placed in the AF tissue surrounding the VA. The limbs of the sutures were approximately 6 mm apart. A vertical suture (VS), without an anulotomy, was placed in the same IVD posterior to the vertical suture surrounding the HA and a horizontal suture (HS), without an anulotomy, was placed posterior to the horizontal suture surrounding the VA. The limbs of the sutures placed in the posterior lateral portion of the IVD were approximately 3 mm apart. The locations of the horizontal and vertical anulotomies were alternated between the left and right sides of the spines at successive IVDs.

The spine sections were mounted on an Instron machine and the sutures were pulled at a rate of 20 mm/sec. The maximal force required to pullout each of the 28 suture loops in the 7 IVDs was recorded.

TABLE I

| Level | HA (N) | HA (mm) | VA (N) | VA (mm) | HS (N) | HS (mm) | VS (N) | VS (mm) |
|---|---|---|---|---|---|---|---|---|
| T9/10 | 175.1 | 5.3 | 125.1 | 5.5 | 97.3 | 2.7 | 61.2 | 3.2 |
| T10/11 | 136.7 | 5.3 | 125.4 | 5.9 | 161.4 | 3.3 | 185.2 | 2.7 |
| T11/12 | 191.6 | 4.9 | 195.7 | 5.6 | 169.8 | 5.5 | 197.5 | 2.5 |
| T12/L1 | 317.7 | 5.6 | 216.2 | 8.2 | 78.9 | 3.3 | 257.1 | 2.7 |
| L1/2 | 256.4 | 6.9 | 192.1 | 8.1 | 104.7 | 3.4 | 298.9 | 3.9 |
| L2/3 | 422.7 | 5.2 | 144.6 | 8.3 | 78.9 | 2.4 | 234.5 | 3.5 |
| L4/5 | 280.4 | 6.4 | 248.3 | 6.3 | 136.6 | 3.2 | 245.7 | 3.6 |
| Avg. | 254.37 | 5.7 | 178.2 | 6.8 | 118.23 | 3.4 | 211.44 | 3.2 |

HA = Horizontal Anulotomy, repaired with vertical suture
VA = Vertical Anulotomy, repaired with horizontal suture
HS = Horizontal Suture without anulotomy
VS = Vertical Suture without anulotomy
N = Pullout force in Newtons
Mm = Length of AF tissue between arms of suture

TABLE II
Data Normalized for length of AF tissue between arms of suture

| Level | HA (N/mm) | VA (N/mm) | HS (N/mm) | VS (N/mm) |
|---|---|---|---|---|
| T9/10 | 33.04 | 22.75 | 36.04 | 19.13 |
| T10/11 | 25.79 | 21.25 | 48.91 | 68.59 |
| T11/12 | 39.10 | 34.95 | 30.87 | 79.00 |
| T12/L1 | 56.73 | 26.37 | 23.91 | 95.22 |
| L1/2 | 37.16 | 23.72 | 30.79 | 76.64 |
| L2/3 | 81.29 | 17.42 | 32.88 | 67.00 |
| L4/5 | 43.81 | 39.41 | 46.69 | 68.25 |
| Avg. | 45.27 ± 18.55 | 26.55 ± 7.85 | 35.73 ± 9.04 | 67.69 ± 23.54 |

### Significant Findings (using Normalized Data)

1. As predicted, vertical sutures have substantially higher pullout force than horizontal sutures of the same length. The mean pullout force of Vertical sutures (56.69±23.34 N/mm (HA+VS)) was significantly higher than the mean pullout force of Horizontal sutures (31.14±9.42 N/mm (VA+HS)), p=0.0007. Horizontal sutures were predicted to have 58 percent of the pullout force of vertical sutures of the same length. The 55 percent difference (see above) is quite close to the predicted difference and can be attributed to the small sample (7 IVDs from a single donor) and the variability of biologic specimens.
2. Anulotomy transects AF fibers that course through the tissue adjacent to the anulotomy and thus weakens AF tissue adjacent to defect in the AF. The mean pullout force of suture placed adjacent to anulotomies (35.91±16.78 N/mm (HA+VA)) was significantly lower than the mean pullout of suture bands placed in through the AF without anulotomy 51.71±23.84 N/mm (HS+VS), p=0.027. Sutures used to repair anulotomy have approximately 62 percent of pullout strength of sutures placed through AF uninjured by anulotomy.

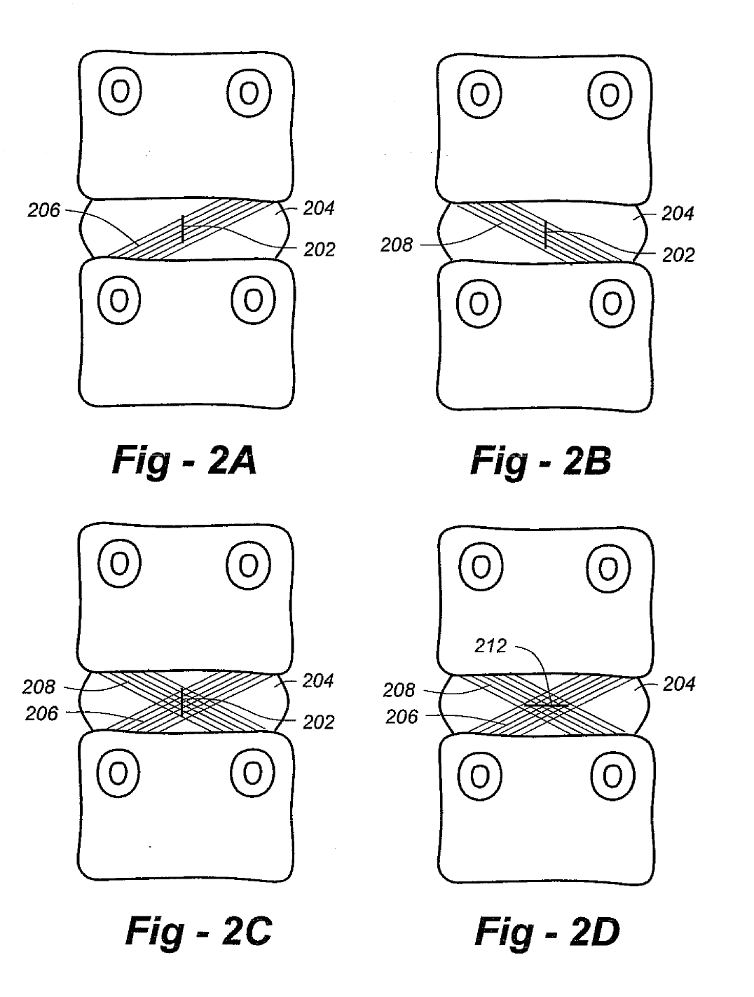

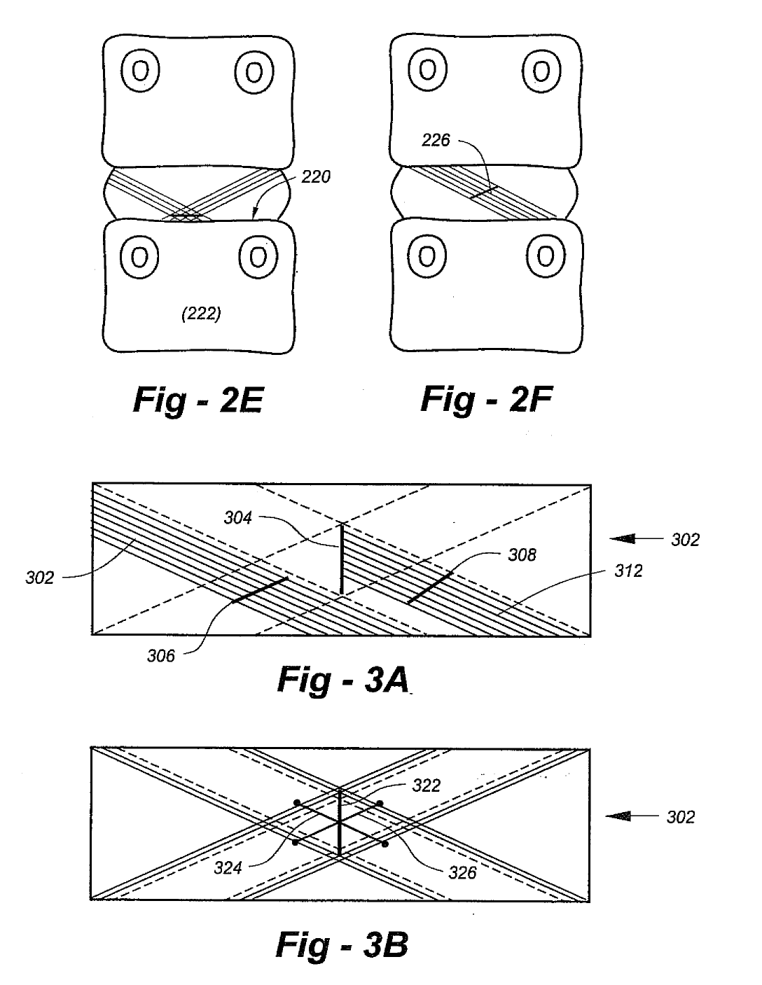

The findings from the above example were used to design inventive devices and methods that take into account the unique structure and physical properties of the IVD. FIG. 2A is a posterior view of a coronal cross section of a portion of the spine. A vertical defect 202 is illustrated in the central portion of the AF 204. Such defects may be created by natural tearing of the AF or by surgical incisions in the AF. The defect transects fibers 206 of each layer of the AF through which the defect extends. The drawing illustrates transection of fibers that pass that previously crossed the defect. The fibers of every other layer of the AF through which the defect extends will be transected in the manner illustrated in the drawing.

FIG. 2B is a posterior view of a coronal cross section of a portion of the spine. The vertical defect 202 is again illustrated in the central portion of the AF. The drawing illustrates transection of fibers 208 that pass that previously crossed the defect. The fibers of every other layer of the AF through which the defect extends will be transected in the manner illustrated in the drawing. The drawing illustrates layers of AF that are adjacent to the layer of AF drawn in FIG. 2A.

FIG. 2C is a posterior view of a coronal cross section of a portion of the spine. The vertical defect is again illustrated in the central portion of the AF. The drawing illustrates transection of fibers 206, 208 of successive layers that pass that previously crossed the defect. All of the fibers of all of the layers of AF through which the defect extends are transected in the diamond shaped area surrounding the defect in the AF. Such areas of the AF are severely weakened by the defect in the AF. Fifty percent of the fibers (100 percent of the fibers coursing in a first direction and 0 percent of the fibers coursing in the alternative direction) are transected in the zones of the AF extending from the central severely injured area (four areas illustrated with diagonal lines in a single direction). The areas of AF with 50 percent fiber injury area moderately weakened. The areas of the AF represented by white triangles external to the injured areas of the AF are not injured by the defect in the AF.

FIG. 2D is a posterior view of a coronal cross section of a portion of the spine. A horizontal defect 212 is illustrated in the central portion of the AF 204. The areas of AF with moderate and severe injuries are smaller, but shaped similar to, the areas of such injury following vertical defects (FIG. 2C).

FIG. 2E is a posterior view of a coronal cross section of a portion of the spine. A horizontal defect is illustrated near the cranial vertebral endplate (VEP) 220 of the caudal vertebra 222. The areas of moderate and severe AF injury are illustrated.

FIG. 2F is a posterior view of a coronal cross section of a portion of the spine. A defect 226 at 60 degrees relative to the vertical axis of the spine is illustrated. The defect creates the moderate injury zone illustrated in the drawing but does not create a severe injury zone.

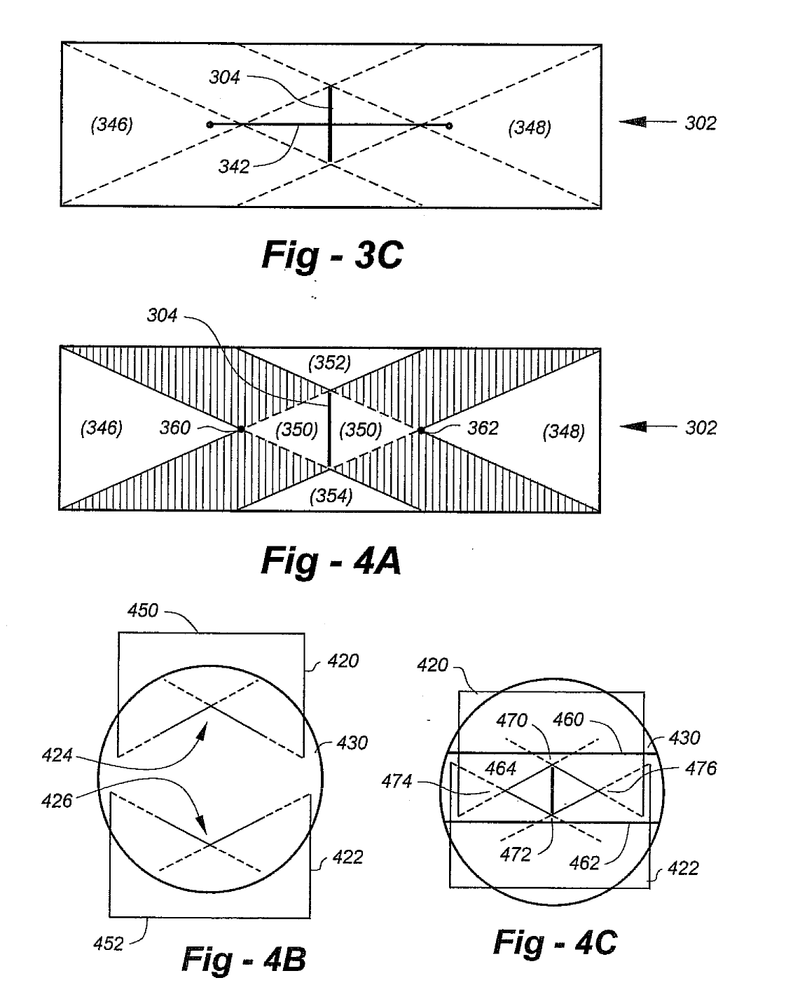

FIG. 3A is a posterior view of an intervertebral disc (IVD) 302. A vertical defect 304 is illustrated in the central portion of the IVD. Two sutures 306, 308 were passed through the AF and tied. The intact AF fibers 310 that course from the upper left hand corner of the drawing to the lower right hand drawing were incorporated in the loop of suture 306 on the left side of the drawing. AF fibers that travel in such direction were also transected by the vertical defect in the AF. The suture loop 308 on the right hand side of the drawing incorporates transected AF fibers 312.

The strength of the connection between successive layers of AF is substantially weaker than the tensile strength of the fibers of the AF. Thus, the force required to pull the suture loop 308 out of the AF on the right of the drawing, which incorporates transected fibers of AF, is substantially lower than the force required to pull the suture loop 306 out of the AF on the left hand side of the drawing, which incorporates intact layers of AF. Studies on horse and cow spines indicate 32 percent higher forces are required to pull out sutures in the configuration on the left side of the drawing than the forces required to pull out sutures oriented as illustrated on the right hand side of the drawing.

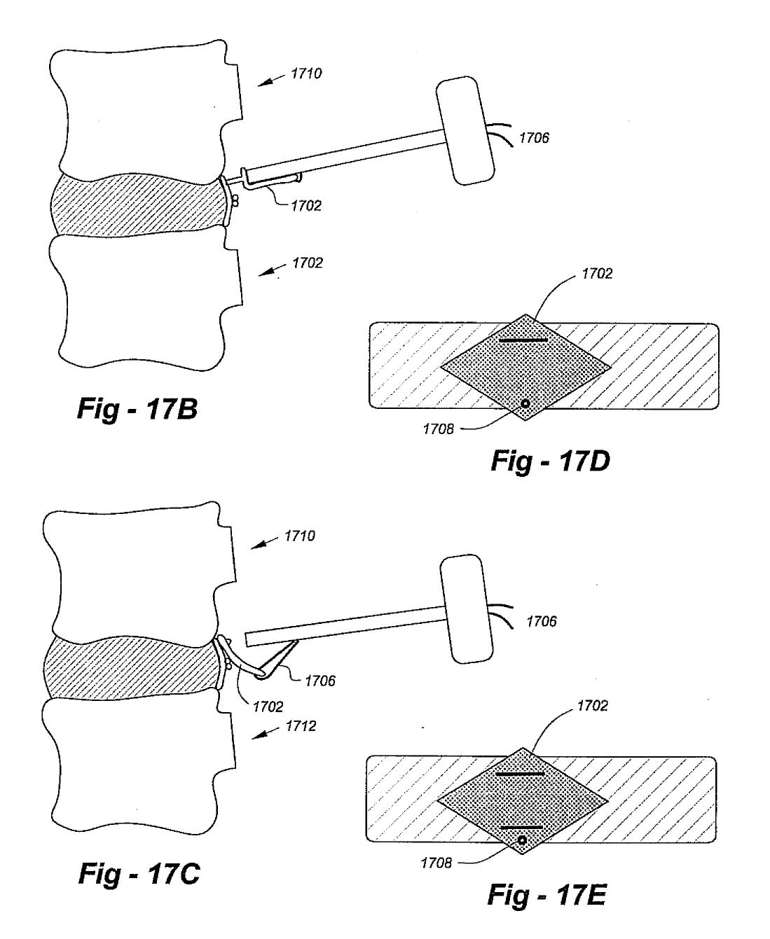

FIG. 3B is a posterior view of IVD 302 and a preferred embodiment of the invention. A vertical defect 304 is again illustrated in the central portion of the IVD. Two sutures 324, 326 were placed in the AF diagonally across the defect (in the manner taught in my co-pending U.S. patent application Ser. No. 11/715,579, FIG. 17B). The sutures 324, 326 pass through the moderately injured zones of the AF. The intact fibers of AF (illustrated by the closely spaced diagonal lines) between the points the sutures pass through the AF and the severely injured zone (central diamond shaped zone illustrated by dotted lines that surround the vertical defect) of the AF resist tensile forces by the sutures.

This, according to the invention, sutures or other fixation members are placed in moderately injured zones of the AF rather than the severely injured regions of the AF. Sutures or other fixation members placed in severely injured areas of the AF are likely to fail as the sutures are easily pulled through the weakened tissue. Pull out of sutures in the moderately injured zones of the AF is resisted by intact fibers of every other layer of AF. Such intact AF fibers course in a single direction. There are no intact AF fibers within the severely injured zone. Sutures or other fixation members may be used to pull AF tissue on either side of the defect together, thus closing the defect or aperture in the AF. Alternatively, sutures or other fixation members may be used to fasten AF repair devices, such as porous mesh to the AF surrounding the defect. Alternatively, sutures or other fixation members may be used to close the aperture in the AF and to fasten anular repair devices to the AF.

FIG. 3C is a posterior view of the AF. The vertical defect 304 is illustrated in the central region of the AF. A suture 342 was passed through uninjured zones 346, 348 of AF. Pull out of sutures in the uninjured zones of the AF is resisted by intact fibers in all layers of the AF. The sutures or other fixation members may be used to pull AF tissue on either side of the defect together, thus closing the defect or aperture in the AF. Alternatively, sutures or other fixation members may be used to fasten AF repair devices, such as porous mesh to the AF surrounding the defect. Alternatively, sutures or other fixation members may be used to close the aperture in the AF and to fasten anular repair devices to the AF.

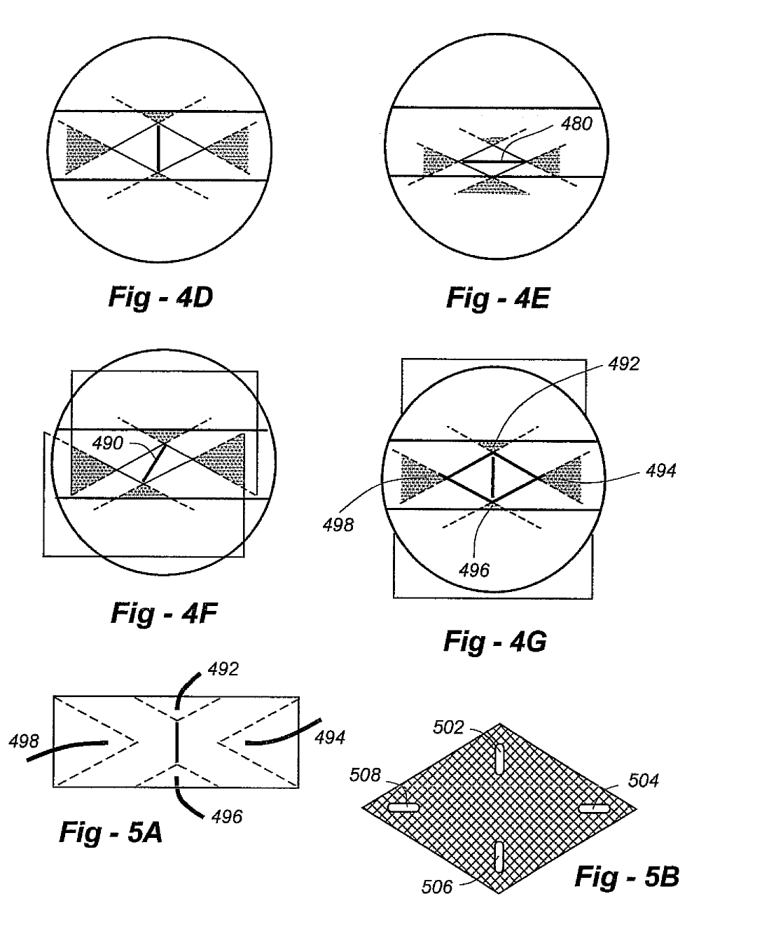

FIG. 4A is a posterior view of the AF showing the zones of injury and uninjured regions in FIGS. 3A-3C. The vertical defect is shown at 304. The white diamond 350 surrounding the vertical defect represents the severely injured zone of the AF. The areas with closely spaced vertical lines represent the moderately injured zone of the AF. The areas of the drawing with dots represent the uninjured zones 346, 348, 352, 354 of the AF. The location of the uninjured zones 352, 254 of AF just beyond the ends of the defect is easy to identify in FIG. 4A. The apex of the triangles of uninjured zones of the AF more distant from the defect (i.e., 360, 362) can be determined with reference to FIGS. 4B-G.

FIG. 4B shows an instrument according to the invention. Components 420, 422 each have a V-shaped notched end 424, 426 that slide into and out of a component 430 that may be circular. The notched end components also slide left and right in the component 430, but do not slide about an axis perpendicular to the plan of the paper. The sliding components 420, 422 are preferably made of clear material such as the clear acetate material used to manufacture templates for prosthetic hip and knee devices.

The notched ends 424, 426 of the sliding components are marked with a dark lines and dots as shown in the drawing. Such dark markings are incorporated in the acetate material similar to the dark markings incorporated in prosthetic hip and knee templates. The sloping sides of the notches of the sliding components are at 30-35 degree angles relative to the horizontal ends 450, 452 of the sliding components. Thus, the sides of the notch form an angle in the range of 110-120 degrees. Alternatively, the sides of the notches could lie at a 25, 26, 27, 28, 29, 36, 37, 38, 39, 40, less than 25, or more than 40 degree angle relative to the horizontal ends of the sliding components. Thus, the sides of such notches could form angles of 100-130 degrees, more or less.

The component 430 is sized to fit over the objective lens of an operating microscope, over the sterile drape that fits over the operating microscope, or within the sterile drape that fits over the operating microscope. Alternatively, the device of FIG. 4B could be incorporated into an operating microscope. For example, the circular component may have a diameter of 4 to 8 centimeters. Alternatively, the circular component may have a diameter of about 3.5, 3.6, 3.7, 3.8, 3.9, 8.1, 8.2, 8.3, 8.4, 8.5, less than about 3.5, or more than about 8.5 centimeters.

The circular component is preferably 3 to 7 millimeters tall and 1 to 3 millimeters thick. Alternatively, the circular component may be about 2.5, 2.6, 2.7, 2.8, 2.9, 8, 9, 10, less than about 2.5, or more than about 10 millimeters tall and about 0.5, 0.6, 0.7, 0.8, 0.9, 3.1, 3.2, 3.3, 3.4, 3.5, less than about 0.5, or more than about 3.5 millimeters thick. The sliding components 420, 422 are preferably about 3 to about 7 centimeters wide and about 3 to about 7 centimeters long. Alternatively, the sliding components could be about 2.5, 2.6, 2.7, 2.8, 2.9, 7.1, 7.2, 7.3, 7.4, 7.5, less than about 2.5, or more than about 7.5 centimeters wide or long. The lines used to form the non-notched portions of the sliding component in the drawing do not include dark markings and thus the non notched sides of the sliding components are not visible through the operating microscope.

FIG. 4C is a posterior view of an IVD and the embodiment of the invention drawn in FIG. 4B. The horizontal lines 460, 462 across the middle of the circular component represents the posterior portion of an IVD. The vertical line 464 in the center of the IVD represents an anular defect. The sliding components 420, 422 were moved towards each other until the apex of the notches appeared to contact the top and the bottom of the anular defect. The triangular areas formed by the dotted lines represent portions of the IVD that were not injured by the defect in the AF. The device is used to identify preferred areas to place fixation devices in the AF or the vertebrae. Such areas are represented by the dotted triangles in FIG. 4A. The drawing shows small triangles 470, 472 intact AF cranial and caudal to the AF defect and large triangles 474, 476 lateral to the severely injured region of the AF. The severely injured portion of the AF is represented by the diamond formed by the dark lines of the notches of the sliding component. The cranial or the caudal sliding component is designed to slide over the caudal or the cranial sliding component respectively.

FIG. 4D is drawing of a surgeon's view through the oculars of an operating microscope and the embodiment of the invention drawn in FIG. 4C. However, the dots between the dashed lines of the sliding component are not visible to surgeons. The dots were added to illustrate the location of the uninjured portions of the AF. Surgeons may use the invention to position fixation components in the triangular shaped areas formed by the dashed lines (areas represented by dots). The trapezoidal areas between the dotted lines represent the moderately injured portions of the AF. In the preferred embodiment, surgeons cannot see the sliding components or the circular ring of the targeting device. Surgeons only see the operative field (IVD and vertebrae) and the markings on the otherwise clear sliding components.

Alternatively, the sliding components could be slightly colored. For example, the cranial sliding component could be light yellow and the caudal component could be light blue. Overlapping portions of the sliding components produce green triangles lateral to the severely injured region of the AF. Lateral fixation components are preferably placed in such green triangles. Alternative colors or markings within the sliding components may be used in the invention. Surgeons preferably place fixation members within uninjured zones of AF at least 1 to 2 millimeters from the apex of such zones. Such placement assures at least 1-2 millimeters of AF tissue with intact fibers that course in both directions.

In FIG. 4E, a horizontal AF defect 480 was drawn near the cranial edge of the caudal vertebra. The sliding components were positioned at the edges of the AF defect. The targeting device indicates relatively large areas of uninjured AF tissue to the left and right of the AF defect and cranial to the AF defect (area with dots). However, the targeting device indicates there is only moderately injured AF caudal to the AF defect. Aided by the targeting device, surgeons may choose to place a fixation member in the caudal vertebra or in the moderately injured zones of the AF caudal to the AF defect. The invention helps surgeons avoid placing fixation components in damaged portions, severe or moderate, of the AF.

In FIG. 4F an inclined AF defect 490 was drawn in the center of the IVD. The cranial sliding component was moved to the right of the drawing or the caudal sliding component was moved to the left of the drawing to align the targeting device with the ends of the AF defect. However, the device prevents rotation of the sliding components about an axis perpendicular to the plane of the paper. The sides of the notches of the sliding components are designed to be parallel to the fibers of the AF. Rotation of the sliding components about an axis perpendicular to the plane of the paper causes the sides of the notches of the sliding components to misrepresent the direction of the fibers of the AF and may lead to placing fixation members into damaged AF tissue.

The “sliding components” could be directed by a mechanism similar to the mechanism used to move slides on the stage of a microscope used to view histology in an alternative embodiment of the invention. Such microscopes uses two controls that turn gears that move the stage at orthogonal angles but do not allow rotation of the stage about an axis perpendicular to the stage. Alternative mechanisms to move the components of the targeting device or alternative targeting devices may be used to identify the various zones of AF injury in alternative embodiments of the invention. Such targeting mechanisms include the projected light, including laser projections that may be positioned on the AF. Alternatively, templates, guides, or measuring devices may be temporarily placed on the AF

The effect of anulotomies or tears on the AF were modeled. The AF was modeled with lamellae fibers organized in alternating configurations at an angle of 60 degrees with respect to the vertical axis. This study did not model incomplete lamellae.

All anulotomies or tears were assumed to pass perpendicularly through all lamellae of the AF, and they were centered on the intervertebral disc (IVD). Anulotomies or tears were made with 4, 8 and 12 mm cuts. Four different types of tears were considered: slits at 0, 30, 60 and 90 degrees. The area of the AF transected by the tear was calculated with moderate injuries having 50 percent or greater of fibers transected, and severe injuries having 100 percent of fibers transected. In addition, the maximal width of the injured section of the AF was calculated.

it was found that there is a linear relationship between length of anular tears and the maximum width of the severe injury zone resulting from the tear (Table III). The maximum width of the severe injury zone perpendicular to the AF defect is 173 percent of the length of vertical tears of the AF (Table III). AF tears parallel to the fibers of the AF, 30 degrees, do not create severely injured areas of AF.

TABLE III

| | 4 mm | 8 mm | 12 mm | Width of severe |
|---|---|---|---|---|
| Cuts | d²(mm) | d²(mm) | d²(mm) | injury zone |
| 0° | 2.3 | 4.6 | 6.9 | 58% |
| 30° | | | | |
| 60° | 6.1 | 12.2 | 18.3 | 153% |
| 90° | 6.9 | 13.8 | 20.8 | 173% |

d² = maximum width of severe (100%) injury zone

Surgeons could use Table III to identify preferred locations to place fixation members in alternative embodiments of the invention. Surgeons could measure the length of the AF defect, estimate the angle of inclination of the defect, and consult the table to determine the area of severe injury. For example, surgeon could place fixation members 1 to 2 millimeters, or more, beyond the ends of a vertical defect and place lateral fixation members the length of the defect lateral to the defect. Such lateral fixation members would be two times the length of the defect apart, thus greater than the 173 percent indicated in Table III.

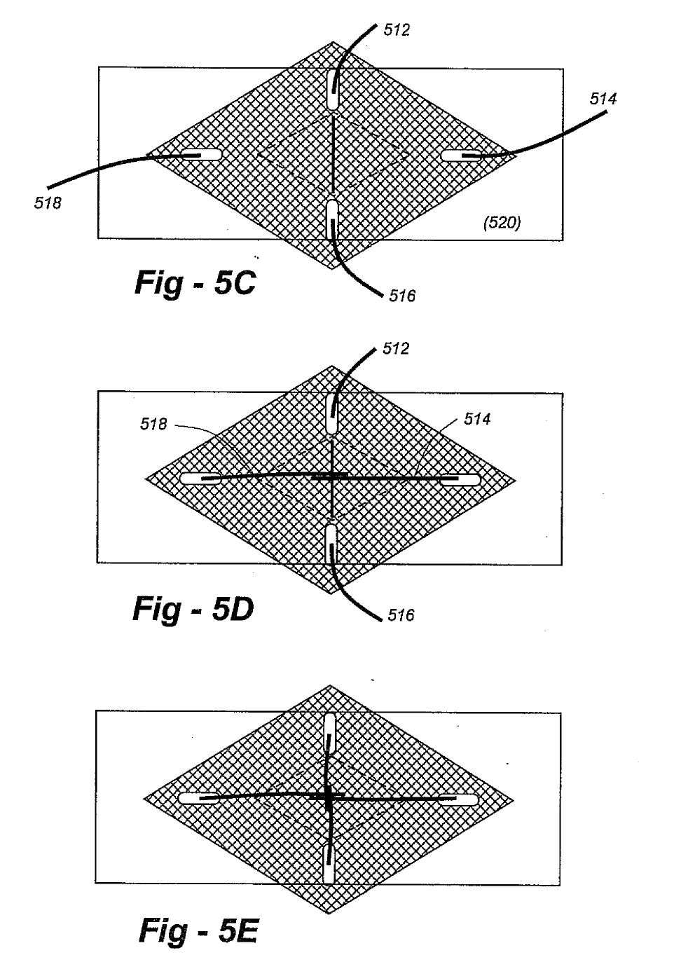

FIG. 4G is view through the oculars of an operating microscope and a posterior view of the embodiment of the invention drawn in FIG. 4D. The targeting device was used to place four fixation members 492, 494, 496, 498 in uninjured zones of the AF that surround a vertical AF defect. The targeting device includes a measurement feature. Intersection of the markings on the sliding components indicates the width of the severely injured zone of the AF. Such measurements help surgeons choose the proper size of mesh reinforcement components such as drawn in FIG. 5B. The targeting device may also include vertical, horizontal, and diagonal scales to assist the surgeon measure the size of the defective region of the AF and the distance between the preferred anchor sites and the edges of the severe AF injury zone.

Alternative measurement devices may be used in the invention to select the proper size of mesh reinforcement components. For example, measurement tools within the operating microscope or laser measuring devices may be used to select the proper size of mesh component. The 30-35 degree angle lines of the targeting device could also guide surgeons to create anulotomies at 30-35 degrees relative to the vertebral endplate of the IVD. Such anulotomies minimize the number of transected AF fibers and do not create severely injured zones in the AF. Surgeons could use such anulotomies to remove NP contained by the AF or to insert prosthetic devices such as total disc replacements, nucleus replacements, spinal cage, or other device.

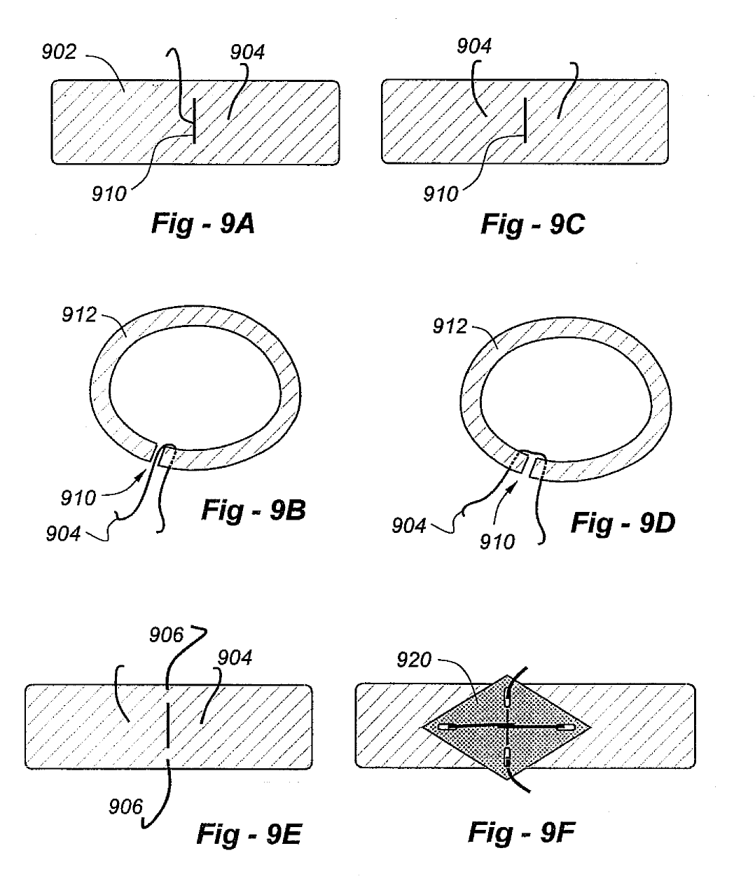

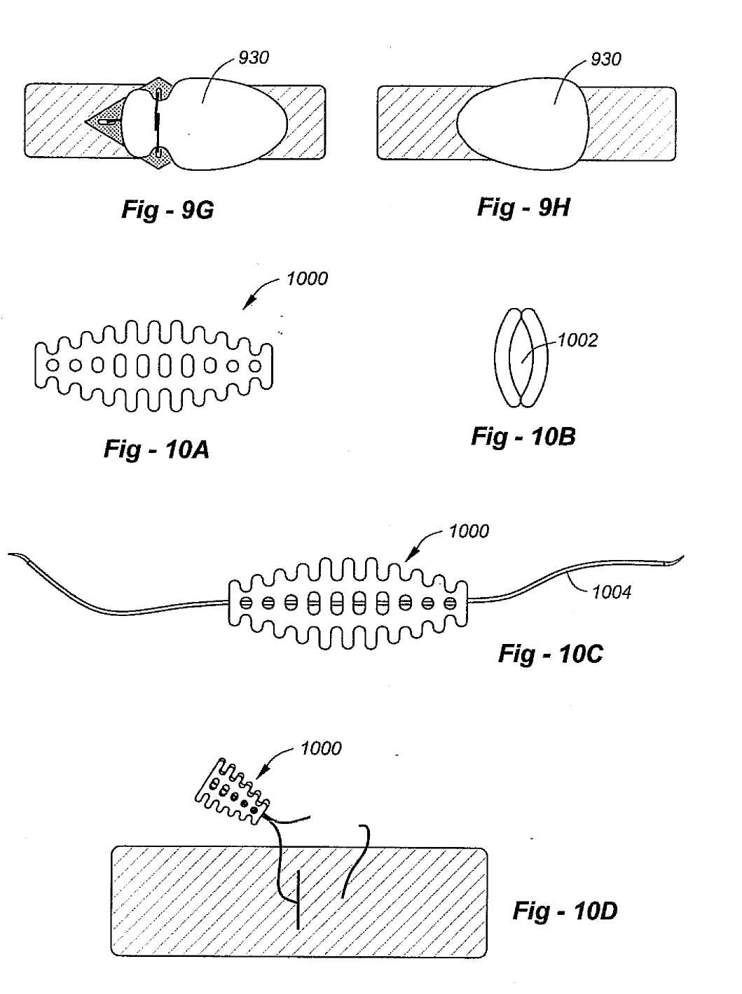

FIG. 5A is a posterior view of the AF. Fixation members were placed in uninjured zones of the AF surrounding a vertical defect in the AF using the embodiments of the invention taught in FIGS. 4A-4G. The triangles outlined by dots are areas of the AF that were not injured by the defect in the AF. The fixation members similar to those taught in co-pending patent applications including FIGS. 9A, 9B and 16A-17B of U.S. application Ser. Nos. 11/708,101 and 11/716,579, which preferably have flexible longitudinal fixation members that extend through the AF and transverse members that lie behind the inner layer of the AF are used in the preferred embodiment of the invention.

Flexible longitudinal fixation members attached to alternative anchor components may alternatively be used in accordance with the invention. For example, anchors that expand in a radial direction or that have elastic components that deploy after placement through a hole in the AF may be used to anchor the flexible longitudinal components. Alternatively, coil shape anchors may be rotated through the AF. Alternative anchors used to attach flexible longitudinal fixation components into soft tissue may be used in alternative embodiments of the invention. For example, such technology may be adapted from devices used in hand, wrist, elbow, shoulder, knee, ankle, or foot surgery. Such anchors are preferably MRI compatible, have a transverse width of about 5 to 10 millimeters when deployed after placement through the AF, have a transverse width of about 1 to 4 millimeters while being pushed through the AF, and have a length of about 1 to 10 millimeters after deployment. Alternatively, such anchors could have a transverse width of about 2, 3, 4, 11, 12, 13, 14, 15, or more millimeters when deployed, a transverse width of about 0.5, 0.6, 0.7, 0.8, 0.9, 4.1, 4.2, 4.3, or more than about 4.3 millimeters while being pushed through the AF and have a length of about 0.5, 0.6, 0.7, 0.8, 0.9, 10,1, 10.2, 10.3, 10.4, or more than about 10.4 millimeters after deployment inside or behind the AF, the anchors preferably have pull strength of about 30 to about 80 pounds when deployed behind AF within the uninjured zones as outlined in FIG. 4A. Alternatively, the anchors could preferably have a pullout force of about 25, 26, 27, 28, 29, 81, 82, 83, 84, 85, less than about 25, or more than about 85 pounds when deployed behind uninjured zones of the AF.

The placement of anchors through the AF in previously uninjured zones of the AF does not convert the AF into an injured zone as used in the description of this invention. The flexible longitudinal fixation components are preferably made of monofilament of multifilament materials such as nylon, polypropylene, polyester, or other biocompatible material. Resorbable materials such as Vicryl or PDS (Ethicon, Summerville N.J.) may be used in alternative embodiments of the invention. The flexible longitudinal fixation members preferably have a diameter between about 0.2 and about 0.7 millimeters, a length of about 10 to about 40 centimeters and a tensile break strength of about 20-80 pounds. Alternatively, the flexible longitudinal fixation members may have a diameter of about 0.1, 0.8., 0.9, or more than about 0.9 millimeters, a length of about 8, 9, 41, 42, 43, less than about 8, or more than about 43 centimeters and a tensile break strength of about 15, 16, 17, 18, 19, 81, 82, 83, 84, 85, less than about 15, or more than about 85 pounds. The fixation members are preferably placed more than about 2 to about 3 millimeters from all edges of the severe AF injury zone. Alternatively, fixation members could be placed about 1, 4, 5, 6, or more millimeters from the closest edge of the severe AF injury zone.

FIG. 5B is posterior view of a diamond-shaped porous mesh component designed to be placed over and reinforce the severely injured zone of the AF. Flexible longitudinal fixation members may be passed through the oval openings 502, 504, 506, 508 at the corners of the device. The device is preferably about 5 to about 20 millimeters wide, about 5 to about 15 millimeters tall, and about 0.2 to about 0.7 millimeters thick. Alternatively, the device could be about 3, 4, 21, 22, 23, or more millimeters wide, about 3, 4, 16, 17, 18, or more millimeters tall, and about 0.1, 0.8, 0.9, or more millimeters thick. The device preferably has interstitial pores that are 0.2-1 millimeter wide by about 0.2-1 millimeters tall. Alternatively, the device may have pores about 0.05, 0.1, 0.15, 1.1, 1.2, 1.3, less than about 0.05, or more than about 1.3 millimeters wide or tall.

The device of FIG. 5B may be made of polyester, polypropylene, expanded polytetrafluorethylene (ePTFE), allograft tissue, xenograft tissue, autograft tissue, combinations of such materials or other biocompatible materials. The oval openings are preferably about 0.5 to 1 millimeter wide and 1 to 3 millimeter long. Alternatively, the oval openings could be about 0.3. 0.4, 1.1, 1.2, less than about 0.3, or more than about 1.2 millimeters wide and about 0.7, 0.8, 0.9, 3.1, 3.2, 3.3, less than about 0.7, or more than about 3.3 millimeters long. The oval openings prevent the flexible longitudinal fixation elements from wrinkling the mesh when tension is applied to the flexible elements to pull the AF tissue together. The AF tissue is pulled together to reduce the size of the aperture in the AF.

FIG. 5C is posterior view of an IVD and the embodiments of the invention drawn in FIGS. 5A and 5B. The ends of the flexible longitudinal fixation components 512, 514, 516, 518 were passed through the oval openings in the mesh component, preferably outside the patient's body as similar to the invention described in co-pending U.S. application Ser. No. 11/805,677. The flexible longitudinal fixation elements guide the mesh patch to the AF 520. The dotted lines outline the edges of the severely injured zone of the AF. The mesh patch preferably extends about 2-4 millimeters beyond the severely injured zone of the AF. Alternatively, the mesh patch could extend about 0.5, 1, 1.5, 4.5, 5, 5.5, 6, less than about 0.5, or more than about 6 mm beyond the serve injury zone. Alternatively, the mesh patch could cover only a portion of the severe injury zone. For example, a rectangular, triangular, hexagonal, round or other shape patch could cover only the central area of the severe injury zone.

FIG. 5D is posterior view of an IVD and the embodiment of the invention drawn in FIG. 5C. The lateral two flexible longitudinal fixation elements 514, 518 were fastened to one another using “knotless fixation” technology. For example, ultrasonic or thermal welding tools (Axya Medical, Beverly Mass.) could be used to weld the flexible components together. Eight to twenty pounds of tension is preferably applied to the ends of the flexible elements before welding them together to pull the two sides of the opening in the AF together. Alternatively, about 6, 7, 21, 22, 23, 24, 25, less than 6, or more than 25 pounds of tension may be applied before fastening the flexible elements to each other or to one or more additional components, such as crimp or deformable component. The high pullout force of the anchors, the high tensile break strength of the flexible longitudinal fixation elements, and the high tension placed on the flexible fixation elements is enabled by the preferred placement of the anchors in uninjured zones of AF tissue. The flexible fixation components decrease the width of the aperture in the AF, pull the AF tissue together, force the mesh patch against the AF, fasten the mesh patch to the AF, and reinforce the mesh patch.

In FIG. 5E, the cranial and caudal fixation elements 512, 516 were fastened together as described in the text of FIG. 5D. Mild tension may be applied to the ends of the flexible elements before fastening such elements to each other, to a third component, or to a locking mechanism in the adjacent anchor. For example, about 3 to I0 pounds of tension may be applied to the flexible elements before fastening them to each other, a third component, or to a locking mechanism in or near an anchor. Alternatively, about 1, 2, 11, 12, 13, 14, or more than about 14 pounds could be applied to such flexible fixation elements before fastening the elements. Excess tension on the flexible fixation elements is avoided to prevent the flexible fixation from enlarging the width of the aperture in the AF.

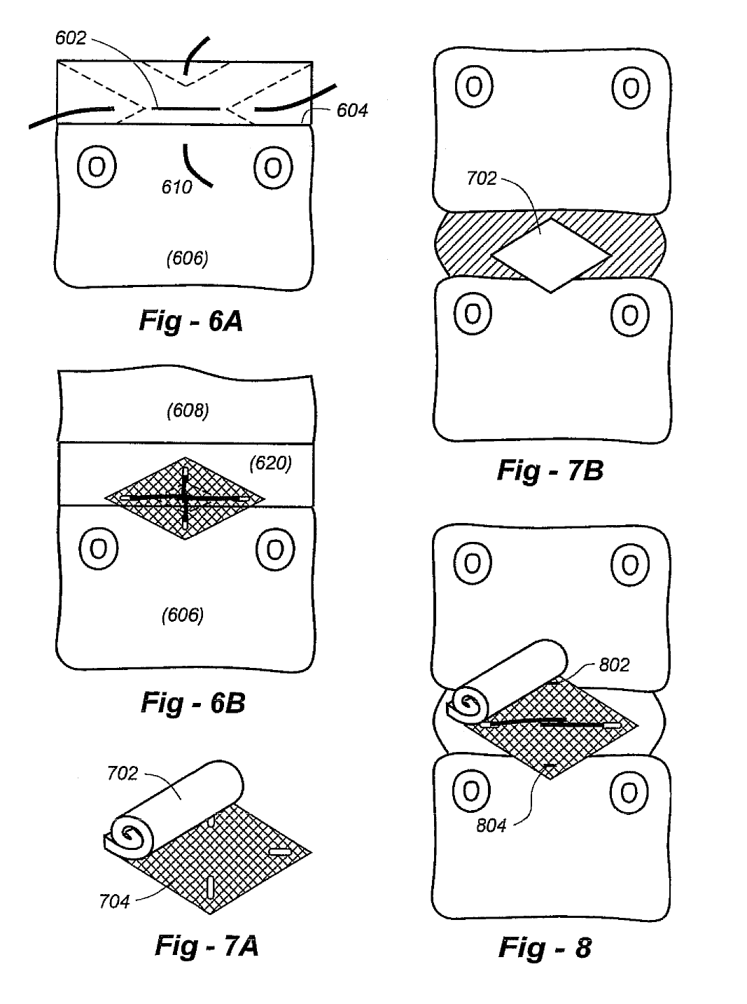

FIG. 6A is a posterior view of an IVD and a caudal cross section of a vertebra. A horizontal AF defect 602 was drawn near the cranial end 604 of the caudal vertebra 606. The targeting embodiment of the invention drawn in FIGS. 4B-G indicated there was no uninjured AF tissue caudal to the AF defect. Consequently, an anchor with a flexible longitudinal fixation element 610 was placed into the caudal vertebra. Bone anchors that expand in a radial direction that have elastic or shape memory components that extend away from the central axis of the anchor after placement of the anchor are preferably used in the vertebra. Such “push in” anchors are generally impacted into bone or holes drilled into bone. Examples of nonscrew in or push in anchors include Impact, UltraFix RC, Ultrafix MiniMite anchors (Conmed, Largo Fla.), Bioknotless, GII, Versalok, Micro, and Super anchors (DePuy Mitek, (Raynham Mass.), Bio-SutureTak (Arthrex Naples, Fla.), and Collared Harpoon and Umbrella Cancellous Harpoon (Arthrotek, Warsaw, Ind.).

Alternatively, as noted in U.S. application Ser. No. 11/635,829 entitled “Sutures for use in the Repair of Defects in the Anulus Fibrosus,” which is hereby expressly incorporated by reference in its entirety, an in-situ curing material, such as a bioactive cement, may be injected into the bone proximal to the anchor to increase the force required to pull the anchor out of the bone. Alternatively, threaded anchors could be screwed into the vertebra of a hole drilled in the vertebra. The anchors are preferably about 2-10 millimeters in diameter and about 5-15 millimeters in length. Alternatively, the anchors could be about 1, 11, 12, 13, or more millimeters in diameter and about 3, 4, 16, 17, 18, or more millimeters in length.

The anchors may be made of titanium, stainless steel, nylon, delrin, PEEK, carbon fiber reinforced PEEK, shape memory material such as Nitinol, or bioresorbable materials such as polylactic acid (PLA), polyglycolic acid (PGA), poly (ortho esters), poly(glycolide-co-trimethylene carbonate), poly-L-lactide-co-6-caprolactone, polyanhydrides, poly-n-dioxanone, and poly(PHB-hydroxyvaleric acid). The sutures or welds are preferably designed to break at a force less than is required to pull the anchors out of the vertebrae. For example, AxyaLoop Ti 3.0 (Axya Medical, Beverly Mass.) suture anchors have a mean pullout force of about 77.9 pounds in cancellous bone. Such anchors could be used with weldable #2 nylon suture (Axya Medical, Beverly Mass.) which has an tensile breakage strength of 20 pounds. Alternatively, absorbable sutures could be used to reduce the risk of anchor expulsion.

FIG. 6B is a posterior view of an IVD 620, coronal cross sections of two vertebrae 606, 608, and the embodiment of the invention drawn in FIG. 6A. The vertical flexible longitudinal fixation elements were fastened to each other under high tension before the lateral flexible longitudinal fixation elements were fastened under low tension. As discussed in the text of FIGS. 5D and 5E, the invention is used to decrease the width of the aperture in the AF.

FIG. 7A is a posterior view of a composite device according to the invention that includes an anti-adhesion cover 702 that is fastened to a portion of the porous mesh 704 as taught in co-pending patent application U.S. Ser. No. 11/635,829. The two components 702, 704, may be glued, welded, sutured, or otherwise fastened. The anti-adhesion component 702 preferably has interstitial pore sizes of 3 microns or less to discourage tissue in-growth. The anti-adhesion component is rolled upon itself to expose the porous mesh. The anti-adhesion component may be made of ePTFE, Seprafilm (Genzyme Corporation, Cambridge Mass.), allograft, or absorbable materials such as oxidized atelocollagen type 1, carboxymethylcellulose, hyaluronic acid, polyethylene glycerol, Coseal (Baxter), Tisseal (Baxter), Floseal (Baxter), Duragen Plus (LifeSciences Corporation), and combinations thereof.

In FIG. 7B, the anti-adhesion cover 702 was placed over the flexible fixation elements and the porous mesh shown in FIG. 6B. The anti-adhesion cover prevents adhesions from forming between the mesh and the nerves within the spinal canal. The anti-adhesion cover is preferably 10 to 20 percent larger than and completely covers the mesh patch. Alternatively, the anti-adhesion cover could be about 5, 6, 7, 8, 9, 21, 22, 23, less than about 6, or more than about 23 percent larger than the mesh patch.

FIG. 8 is a posterior view of a coronal cross section of a portion of the spine including the embodiment of the invention drawn in FIG. 7B. Here, staples, tacks, or other such devices 802, 804 were passed through the caudal and cranial corners of the mesh and into the vertebra to fasten the corners of the mesh to the vertebrae rather than the AF in the manner taught in FIG. 5E. Such tacks preferably expand in a radial direction or have deployable components as described for the suture anchors in the text of FIG. 6A. Alternatively, the threaded tacks may be screwed into the vertebrae. For example, Corkscrew Parachute II tissue anchors (Arthrex, Naples Fla.) could be used in this embodiment of the invention. The tacks are similar in size and may be made of the same materials to the anchors described in the text of FIG. 6A.

FIG. 9A is a posterior view of an IVD 902. A suture 904 was passed through an uninjured portion of the AF aided by the targeting invention drawn in FIG. 4B-G. The other end of the suture passes through the defect 910 in the AF. A suture passing tool may be used to pass and retrieve the suture. For example the Scorpion, Viper, Bankart Viper, Needle Punch II, or Plication Viper from Arthrex (Naples, Florida) could be used to pass the suture through the AF. The suture may have needles on both ends. The suture passing instrument preferably has a component that captures the pointed end of the needle thus protecting the spinal nerves from injury. FIG. 9B is an axial cross section of an IVD and the embodiment of the invention drawn in FIG. 9A. The dotted line indicates the course of the suture through the AF 912.

FIG. 9C is a posterior view of an IVD and the embodiment of the invention drawn in FIG. 9A. The end of the suture that passed through the defect in the AF was reloaded into the suture passing tool and passed through the AF on the other side of the defect. The needle was passed from inside the IVD to the outside of the IVD after placing a portion of the suture passing tool through the defect in the AF. FIG. 9D is an axial cross section of an IVD and the embodiment of invention drawn in FIG. 9C.

In FIG. 9E, a second suture 906 was passed through the uninjured portions of the AF beyond the ends of the defect in the AF using the technique described in FIG. 9A-D. FIG. 9F is a posterior view of an IVD and the embodiments of the invention drawn in FIGS. 5D and 9A-E. The ends of the horizontal sutures were welded or otherwise fastened together under tension over a mesh patch 920.

FIG. 9G is a posterior view of an IVD and the embodiment of the invention drawn in FIG. 9F showing how the ends of the vertical suture may be fastened over a portion of an anti-adhesion cover 930. FIG. 9H is a posterior view of an IVD and the embodiment of the invention drawn in FIG. 9G. The anti-adhesion cover was folded over the sutures and mesh.

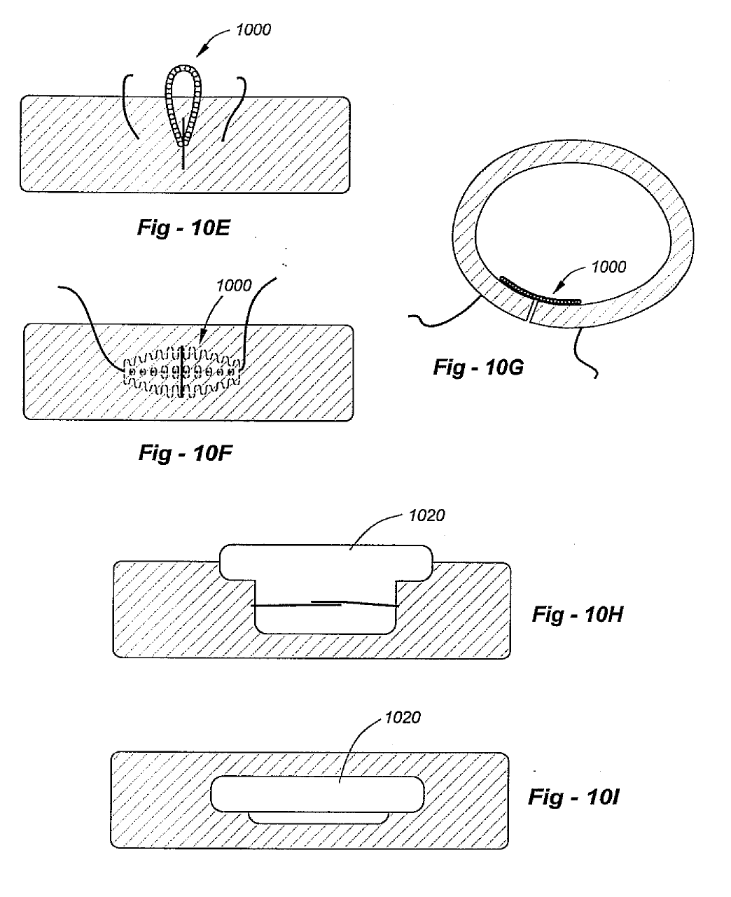

FIG. 10A is a posterior view a flexible reinforcement member 1000 according to the invention that is preferably elastic and which may have shape-memory properties. The component may be made of Nitinol, polyethylene, polypropylene, polyester, titanium, stainless steel, nylon, allograft, xenograft, or other biocompatible material. The reinforcement member is preferably diamond shaped, about 8 to about 16 millimeters wide, about 4 to about 16 millimeters high, and about 0.25 to about 4 millimeters thick. Alternatively, the reinforcement member may be about 4, 5, 6, 7, 17, 18, 19, 20, less than about 4, or more than about 20 millimeters wide, about 2, 3, 17, 18, 19, or more than about 19 millimeters tall, and about 5, 6, 7, less than about 0.25, or more than about 7 millimeters thick. The reinforcement member may be square, rectangular, circular, hexagon, trapezoid, or other shape in alternative embodiments of the invention.

FIG. 10B is an end view of the device drawn in FIG. 10A. A lumen 1002 courses through the device. FIG. 10C is a posterior view. A suture 1004 was passed through the lumen of the reinforcement member. FIG. 10D is a posterior view of an IVD and the device drawn in FIG. 10C. The first end of the suture was passed through the uninjured or moderately injured zone of the AF using the apparatus and methods shown in FIGS. 9A-B.

In FIG. 10E, the device drawn in FIG. 10A is folded, compressed, or shown in its first collapsed shape. The drawing shows the side of the side of the folded reinforcement member. The second end of the suture was passed through the AF using the embodiment of the invention drawn in FIGS. 9C-D.

FIG. 10F is a posterior view of an IVD and the embodiment of the invention drawn in FIG. 10E. The reinforcement device is represented by the dotted lines and lies behind the AF. The reinforcement device is seen in its expanded, second shape. The collapsed reinforcement may be pushed through the defect in the AF with an instrument. The instrument may also compress and collapse the device. Tension on the ends of the suture helps pull the collapsed reinforcement device through the defect in the AF. The reinforcement device may change to its expanded shape as the compression is released from the device or in a reaction to increased temperature or other environmental change, or compression is released from the device and temperature increases. The reinforcement device may also expand spontaneously after it passes through the AF and thus, compression by the AF is released. FIG. 10G is an axial cross section of an IVD and the embodiment of the invention drawn in FIG. 10F.

FIG. 10H is a posterior view of an IVD and the embodiment of the invention drawn in FIG. 10G. The ends of the sutures were welded or otherwise fastened together, preferably with knotless fixation technology over a portion of an anti-adhesion cover 1020. Tension on the fastened suture holds the reinforcement device against the AF, prevents folding of the expanded reinforcement device, and may help pull AF tissue into the aperture. In FIG. 10I, the anti-adhesion cover was folded over the sutures.

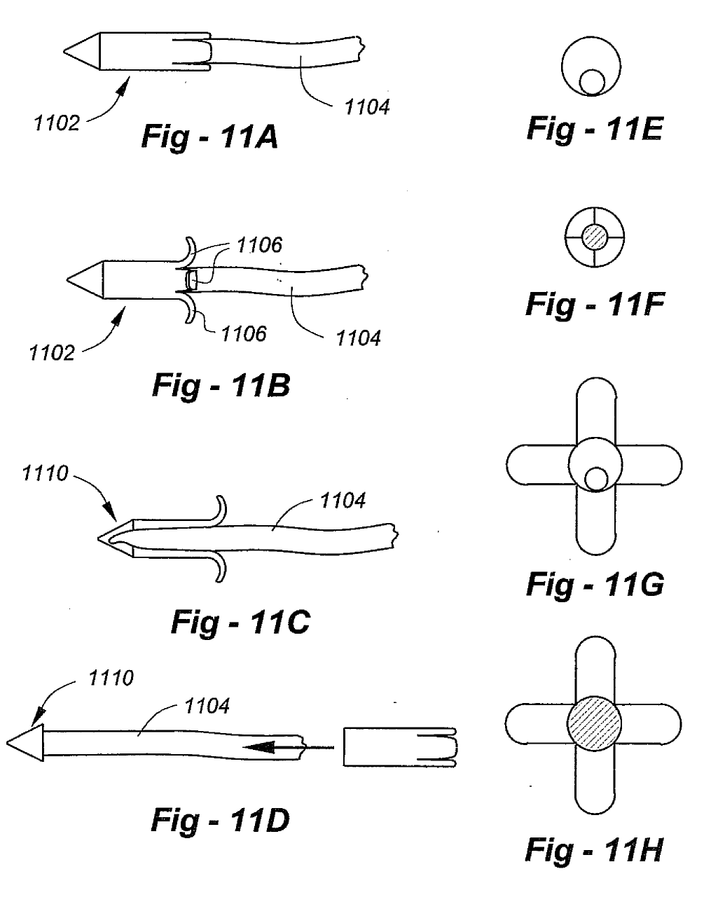

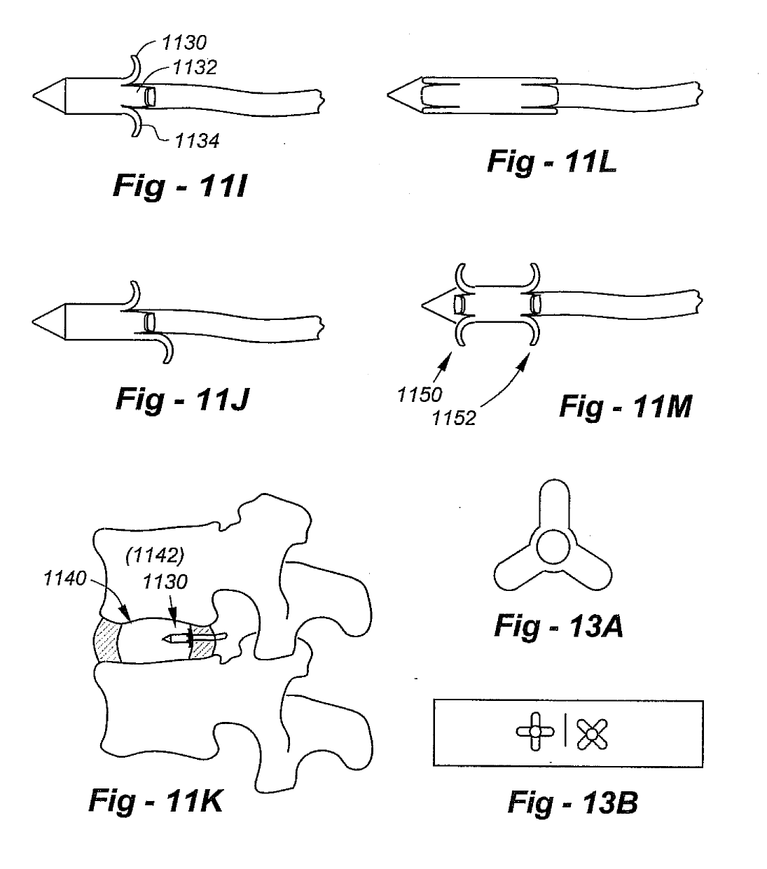

FIG. 11A is a lateral view of an anchor or fixation member 1102 and a portion of a flexible longitudinal fixation element 1104. The embodiment of the invention is also described in the text of FIG. 5A. The anchor was drawn in its contracted shape. Such shape facilitates insertion of the anchor through a small hole or defect in the AF and into a hole in the vertebra.

FIG. 11B is a lateral view of the embodiment of the invention drawn in FIG. 11A. The fixation member was drawn in its expanded shape. The arms 1106 of the fixation member expand away from the flexible longitudinal fixation element after the fixation member is passed through a defect in the AF or into a hole in the vertebra. The fixation member is preferably made of a shape-memory material such as Nitinol.

The arms 1106 may expand after the device is pushed through a cannula, or secondary to a change in temperature, or both elastically and secondary to martensitic transformation of the shape memory material. Fixation members designed for use in the AF are preferably 6-10 millimeters long when the device is contracted, have a contracted diameter of 1 to 2 millimeters, and an expanded diameter of 7 to 9 millimeters. Alternatively, fixation members for use in the AF are preferably 3, 4, 5, 11, 12, 13, or more millimeters long when the device is contracted, have a contracted diameter of 0.5, 0.6, 0.7, 0.8, 0.9, 2,1, 2.2, 2.3, 2.4, 2.5, or more millimeters, and an expanded diameter of 4, 5, 6, 10, 11, 12, or more millimeters. The width of the fixation member preferably increases 400 to 800 percent as the device changes from its contracted to its expanded shape. Alternatively, the width of the fixation member may increase 100, 200, 300, 900, or more percent as the device changes from its contracted to its expanded shape.

Fixation members designed for insertion into the vertebrae preferably have larger contracted diameters than fixation members designed for insertion through defects in the AF. For example, fixation members designed for insertion into the vertebrae may have preferred contracted diameters of 1.7 to 4 millimeters. Alternatively, the vertebral fixation members could have contracted diameters of 1, 1.1, 1.2, 1.3, 1.4, 1.5, 1.6, 4.1, 4.2, 4.2, 4,4, or larger than 4.4 millimeters. The flexible longitudinal fixation elements preferably have a diameter of 0.4 to 1.0 millimeters and a length of 10 to 30 centimeters. Alternatively, the flexible longitudinal fixation elements may have diameters of 0.2, 0.3, 1.1, 1.2, 1.3, or more than 1.3 millimeters and lengths of 8, 9, 31, 32, or less than 8, or more than 32 centimeters.

The flexible longitudinal fixation elements are preferably made of multifilament materials such as polyester, or monofilament materials such as nylon or polypropylene. The flexible longitudinal fixation elements are preferably made of permanent materials such as polyester or absorbable materials such as PDS (Ethicon, Summerville N.J.). The flexible longitudinal fixation elements preferably have a tensile break strength of 20 to 80 pounds. Alternatively, the flexible longitudinal fixation elements could have a tensile break strength of 15, 16, 17, 18, 19, 81, 82, 83, 84, less than 15, or more than 84 pounds. The arms of the device may be fenestrated or treated with a bone growth promoting substance such as hydroxyappetite, plasma sprayed titanium, bone morphogenetic protein, or other material to facilitate fixation of the device to the vertebrae or the AF.

FIG. 11C is a longitudinal cross section of the embodiment of the invention drawn in FIG. 11B. The distal end of the flexible longitudinal fixation element is preferably embedded in the cone-shaped plastic tip 1110 of the device. The plastic tip may be made of nylon, polypropylene, delrin, or other biocompatible material. Alternatively, the distal end of the flexible longitudinal fixation element could fastened to the cone component be crimping the cone component around the flexible fixation element, looping the flexible fixation around a transverse axle-like component across the fixation element or passing the flexible fixation element through a hole in the fixation element.

FIG. 1D is an exploded lateral view of the embodiment of the invention drawn in FIG. 11C. FIG. 11E is a view of the distal, cone, end of the embodiment of the invention drawn in FIG. 11C. The device is drawn in its contracted shape. FIG. 11F is a view of the proximal end of the embodiment of the invention drawn in FIG. 11C. The device is drawn in its contracted shape. FIG. 11G is a view of the distal end of the embodiment of the invention drawn in FIG. 11C. The device is drawn in its expanded shape. FIG. 11H is a view of the proximal end of the embodiment of the invention drawn in FIG. 11C. The device is drawn in its expanded shape. The flexible longitudinal fixation element is seen in cross section.

FIG. 11I is a lateral view of an alternative anchor configuration where pairs of orthogonal arms 1130, 1132 are of different lengths. For example, the vertical arms of the device may be shorter than the horizontal arms. Such configuration facilitates placement of devices near the vertebral endplates. The vertical arms are preferably 2-4 millimeters shorter than the horizontal arms. Alternatively, the vertical arms may be 0.5, 0.75, 1, 4.5, 5, less than 0.5, or more than 5 millimeters shorter than the horizontal arms. The device may be rotated to make the vertical arms longer than the horizontal arms.

FIG. 11J is a lateral view of an alternative embodiment of the invention drawn in FIG. 11I. The arms 1130, 1132, 1134 of the device are of at least three different lengths. The invention facilitates placement of devices near the vertebral endplates and along the curved surface of the inner AF. The arms of the device preferably differ in length by 1 to 5 millimeters. Alternatively, the arms may differ in length by 0.5, 0.6, 0.7, 0.8, 0.9, 5.1, 5.2, 5.3, less than 0.5, or more than 5.3 millimeters.

FIG. 11K is a partial sagittal cross section of a portion of the spine and the embodiment of the invention drawn in FIG. 11J. The short cranial arms 1130 of the device facilitate placement of the device near tile caudal vertebral endplate 1140 of the cranial vertebra 1142.

FIG. 11L is a lateral view of an alternative embodiment of the invention drawn in FIG. 11A. The device is drawn in its contracted shape. FIG. 11M is a lateral view of the embodiment of the invention drawn in FIG. 11L. The device is drawn in its expanded shape. The distal expanded arms 1150 stiffen the proximal expanded arms 1152 if the proximal set of arms bends into the distal set of arms. The proximal set of arms may be relatively flexible until they impinge against the distal set of arms.

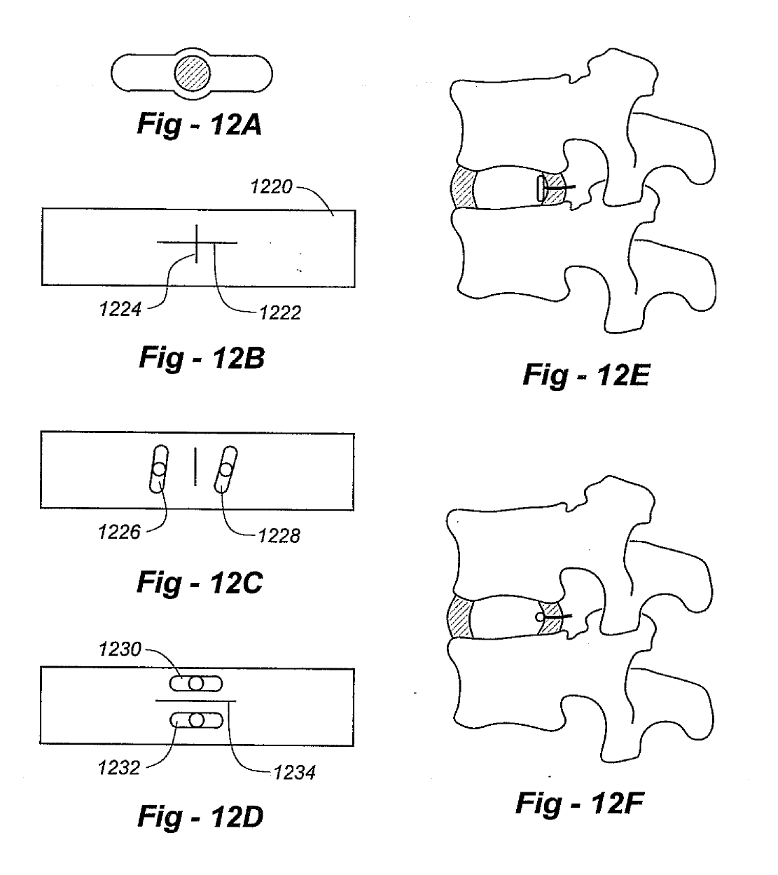

FIG. 12A is a view of the proximal end of an alternative configuration of the device drawn in FIG. 11H. The device has two, linear, expandable arms. The use of such devices is preferably limited to insertion through the AF near the attachment of the AF to the vertebra. Devices with linear arms, expandable or non-expandable, can rotate from a vertical orientation relative to the vertical axis of the spine to a horizontal or 60-degree angle relative to the vertical axis of the spine. As demonstrated in the example discussed in the text of FIG. 1E, anchors members oriented in the horizontal direction have 58 percent of the pullout force of anchors oriented in the vertical direction. In fact, the natural bulging of the AF will cause linear anchors to rotate to the weak horizontal orientation.

FIG. 12B is a posterior view of an IVD 1220. A horizontal suture 1222 was used to repair a vertical defect 1224 in the AF. As discussed in the text of FIG. 1E, horizontal sutures provide only 55-58 percent of the resistance to pullout that vertical sutures of similar length provide. However, vertical sutures cannot be used to repair vertical defects in the AF.

FIG. 12C is a view of the inner portion of the posterior AF. Anchors 1226, 1228 with linear arms, and taught in FIG. 12C, were placed on either side of a vertical defect in the AF. The vertical orientation of the arms of the anchors gain the superior pullout resistance provided by vertical sutures. Thus, such anchors connected by horizontal flexible longitudinal fixation elements (not shown) can be used with vertical AF defects and have the resistance to pullout provide by vertical sutures.

FIG. 12D is a view of the inner portion of the posterior AF. Anchors with linear arms 1230, 1232, and taught in FIG. 12C, were placed cranial and caudal to a horizontal AF defect 1234. The arms of the anchors were rotated to a horizontal orientation. Such rotated anchors may provide the pullout resistance of weaker, horizontal oriented sutures. Horizontal anchors may have less pullout resistance than a vertically oriented suture.

FIG. 12E is a partial sagittal cross section of a portion of the spine and the embodiment of the invention drawn in FIG. 12C. The vertical arms of the anchor do not conform to the natural bulge of the AF.

FIG. 12F is a partial sagittal cross section of a portion of the spine and the embodiment of the invention drawn in FIG. 12E. The arms of the device were rotated 90 degrees. Such rotation will likely occur as the device attempts to conform to the AF. The horizontal orientation of the arms of the anchor is undesirable. Horizontal arms of the device have less resistance to pullout than vertical arms. Furthermore, such rotation may allow the anchor to migrate in a posterior direction, thus loosening the flexible longitudinal fixation element.

FIG. 13A is a view of the proximal end of an alternative embodiment of the invention drawn in FIG. 12A. The device has three expandable arms. Alternatively, the device may have 1, 5, 6, or more expandable arms. Preferred devices with three or more arms that extend in three or more directions resist dissection between the fibers of the AF. Devices such as that drawn in FIG. 12A risk dissection between fibers of the AF particularly in the moderate injury zone of the AF. Furthermore, devices with nonlinear arms cannot rotate into an undesirable, weak, horizontal only orientation.

FIG. 13B is a posterior view of the inner portion of the AF and the embodiment of the invention drawn in FIG. 11G. Such non-linear arms cannot rotate into the weaker horizontal only orientation seen in FIG. 12D.

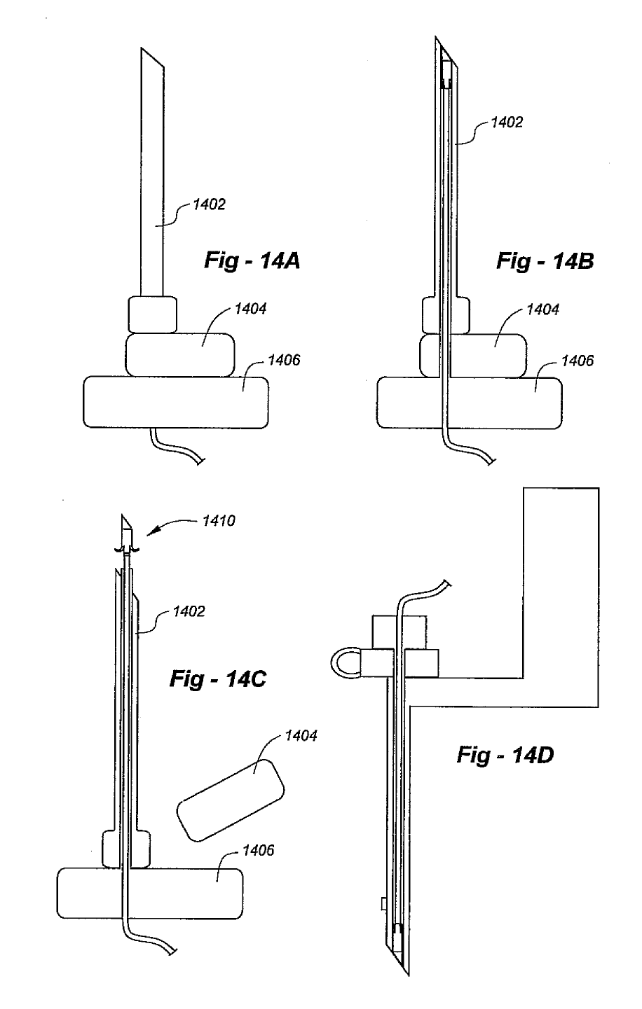

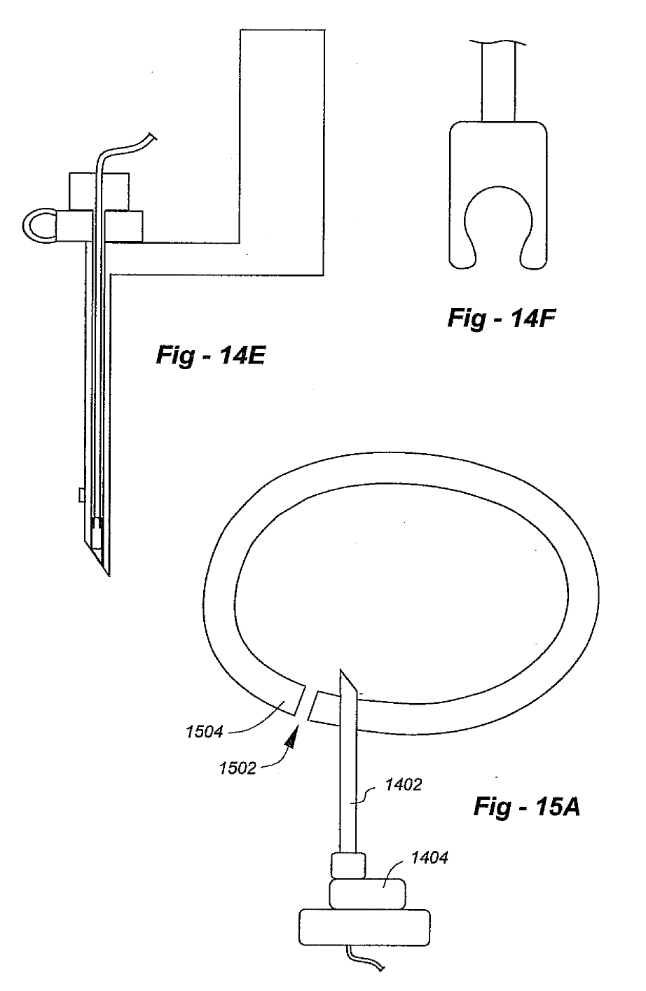

FIG. 14A is a lateral view of the embodiment of the invention drawn in FIG. 11A and a tool used to insert the device into the spine. The embodiment of the invention drawn in FIG. 11A is in its contracted shape inside the tool.

FIG. 14B is a longitudinal cross section of the embodiment of the invention drawn in FIG. 14A. The tool has three components: 1) a beveled cannulated distal component 1402, 2) a spacer component 1404, and 3) a pusher component 1406. The slotted spacer component 1404 is snapped over the side of the pusher component 1406. The flexible longitudinal fixation element extends from the fixation member through the insertion tool. The slope of the cone of the fixation member is preferably the same slope as the bevel of the distal tip of the insertion tool.

The beveled distal component preferably has an outer diameter of 1 to 2 millimeters, an inner diameter slightly larger than the outer diameter of the fixation member, and a length of 15-25 centimeters. Alternatively, the beveled distal component may have an outer diameter of 0.7, 0.8, 0.9, 2.1, 2.2, 2.3, 2.4, less than 0.7, or more than 2.4 millimeters, and a length of 10, 11, 12, 13, 14, 26, 27, 28, or more than 28 centimeters. The spacer component is preferably 1 to 2 millimeters longer than the fixation element and a width of 2-3 centimeters. Alternatively, the spacer component may be the same length as the fixation member or 3, 4, or more millimeters longer than the fixation element. The shaft of the pusher component preferably has an i.d. slightly small than the o.d. of the fixation element and is 1 to 2 millimeters longer than the beveled component. Alternatively, the shaft of the pusher component may be 3, 4, or more millimeters longer than the length of the beveled component. The pusher component has handle that is approximately 5 by 3 by 2 centimeters.

FIG. 14C is an exploded longitudinal cross section of the embodiment of the invention drawn in FIG. 14B. The spacer component 1404 was removed, and the pusher component 1406 was advanced within the beveled component 1402. The fixation member 1410 was forced from the tool. The arms of the fixation member expanded as it was forced the tool. The arms expanded when the pressure from the beveled component was released or secondary to temperature change or both.

In FIG. 14D, the beveled component is shaped different than the beveled component in FIG. 14A to facilitate use of the instrument under an operating microscope. FIG. 14E is a longitudinal cross section of the embodiment of the invention drawn in FIG. 14D. FIG. 14F is a view of the top of the spaced component drawn in FIG. 14E.

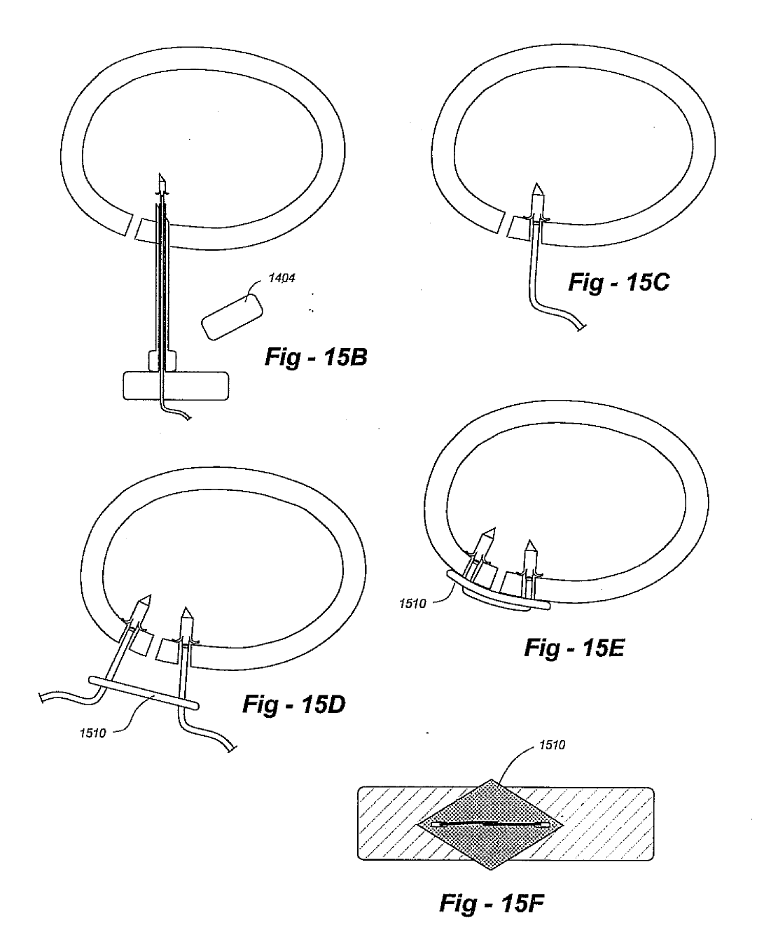

FIG. 15A is an axial cross section of an IVD and the embodiment of the invention drawn in FIG. 14A. The beveled end 1402 of the tool was forced through the uninjured AF adjacent to a defect 1502 in the AF 1504. The outer diameter of the beveled component preferably increases by 1-3 millimeters 7 to 15 millimeters proximal to the tip of the bevel. Alternatively, the outer diameter of the beveled component could increase by 4, 5, 6, or more millimeters 4, 5, 6, 16, 17, 18, or more millimeters proximal to the tip of the bevel. The larger diameter of the beveled component acts as stop to prevent inserting the tool into the IVD too far.

FIG. 15B is an exploded axial cross section of an IVD and the embodiment of the invention drawn in FIG. 15A. The spaced component was removed from the tool, and the fixation member was forced into the IVD, and the arms of the fixation member have expanded.

FIG. 15C is an axial cross section of an IVD and the embodiment of the invention drawn in FIG. 15B. The insertion tool was removed. The length of the expanded arms of the fixation member is longer than the diameter of the opening created in the AF by the insertion tool. The expanded arms of the fixation device preferably contact 4 mm square millimeters to 7 square millimeters (mm²) of area of the inside of the AF. Alternatively, the expanded arms of the fixation device could contact an area of 2.5, 3, 3.5, 7.5, 8, 8.5, or more square millimeters (mm²) of the inner surface of the AF.

In FIG. 15D a second fixation member was inserted into the IVD. The proximal ends of the flexible longitudinal fixation elements were threaded through openings in a mesh patch 1510. FIG. 15E is an axial cross section of an IVD and the embodiment of the invention drawn in FIG. 15D. Tension was applied to the ends of the flexible longitudinal fixation elements and the flexible elements were welded, otherwise fastened to each other, or fastened to the mesh patch. The flexible longitudinal fixation elements pull the sides of the AF surrounding the defect together. The mesh patch 1510 provides a bridge or scaffold for tissue to grow across the defect in the AF. FIG. 15F is a posterior view of an IVD and the embodiment of the invention drawn in FIG. 15E.

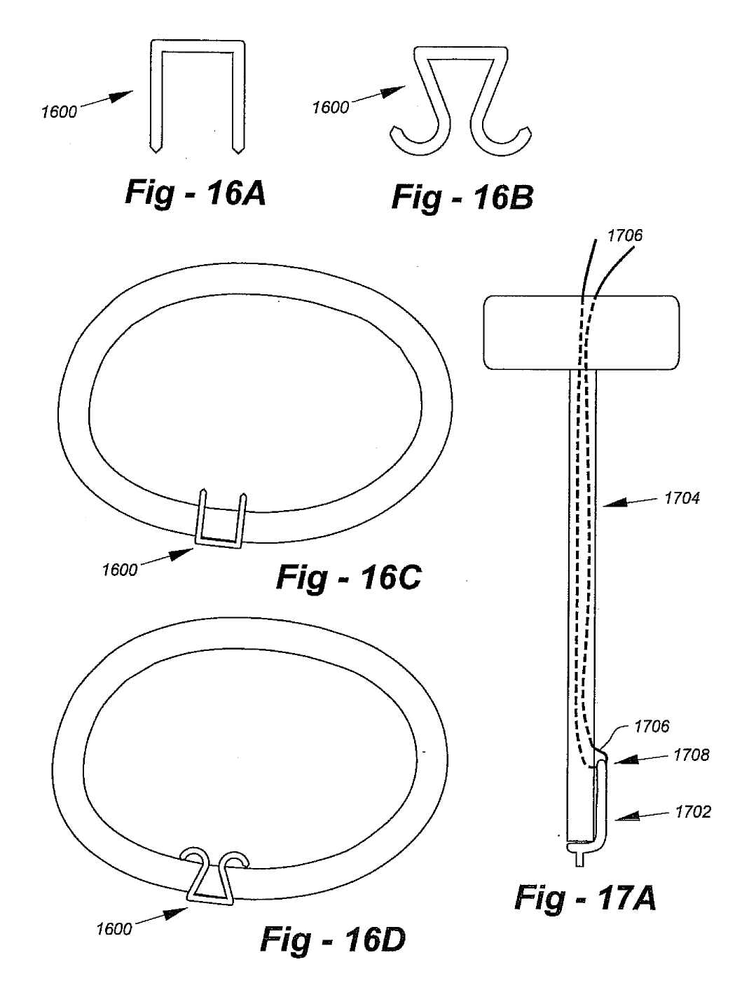

FIG. 16A is a lateral view of a staple-like fixation member 1600 according to the invention drawn in its first shape. The device is preferably made of a shape-memory material such as Nitinol. The device is preferably 1.5 to 3 millimeters wide and 5 to 10 millimeters long. Alternatively, the device may be 1, 4, 5, or more millimeters wide and 3, 4, 11, 12, 13, or more millimeters long. FIG. 16B is a lateral view of the embodiment of the invention drawn in FIG. 16A. The fixation device is drawn in its second shape.

FIG. 16C is an axial cross section of an IVD and the embodiment of the invention drawn in FIG. 16A. The legs of the device were pushed through the AF 1610. FIG. 16D is an axial cross section of an IVD and the embodiment of the invention drawn in FIG. 16B. The legs of the device change to the second shape after the legs are pushed through the AF. The tips of the distal ends of the arms of the device preferably rest against the inner portion of the AF. The distal ends of the device preferably move towards each other as the device assumes its second shape.

FIG. 17A is a lateral view of the embodiment of the invention drawn in FIG. 16A, an anti-adhesion cover 1702, and a tool 1704 used to insert the embodiment of the invention drawn in FIG. 16A. The legs of the fixation device were passed through the anti-adhesion cover. A suture 1706 was passed through a hole 1708 in the opposite end of the anti-adhesion cover and through the shaft of the insertion tool. The anti-adhesion cover is preferably made of ePTFE or other material known to reduce the severity of adhesions.

FIG. 17B is a lateral view of a portion of the spine and the embodiment of the invention drawn in FIG. 17A. The vertebrae were transected through the pedicles 1710, 1712. The posterior elements of the vertebrae, posterior to the pedicles were not drawn. The legs of the staple-like fixation device were partially placed through the mesh patch and the AF.

FIG. 17C is a lateral view of a portion of the spine and the embodiment of the invention drawn in FIG. 17B. The legs of the staple-like device were passed through the mesh patch and the AF. The anti-adhesion cover has been partially released from the tool by pulling the tool over the suture.

FIG. 17D is a posterior view of the IVD and the embodiment of the invention drawn in FIG. 17C. The anti-adhesion cover was released from the tool and covers the mesh patch and the welded sutures. The staple-like fixation device is seen at the cranial end of the anti-adhesion cover. The hole through which the suture passed is seen at the caudal end of the anti-adhesion cover.

FIG. 17E is a posterior view of the IVD and the embodiment of the invention drawn in FIG. 17D. A second staple-like device was placed through the anti-adhesion cover and the mesh patch. The staple-like devices fasten the anti-adhesion cover to the IVD, the corners of the mesh patch to the IVD and may pull the AF tissue on either side of the defect in the AF together. The anti-adhesion cover is preferably 2-3 millimeters wider in each direction than the mesh patch. Alternatively, the anti-adhesion cover may be 1, 4, 5, 6, or more millimeters wider than the mesh patch in one or more directions.

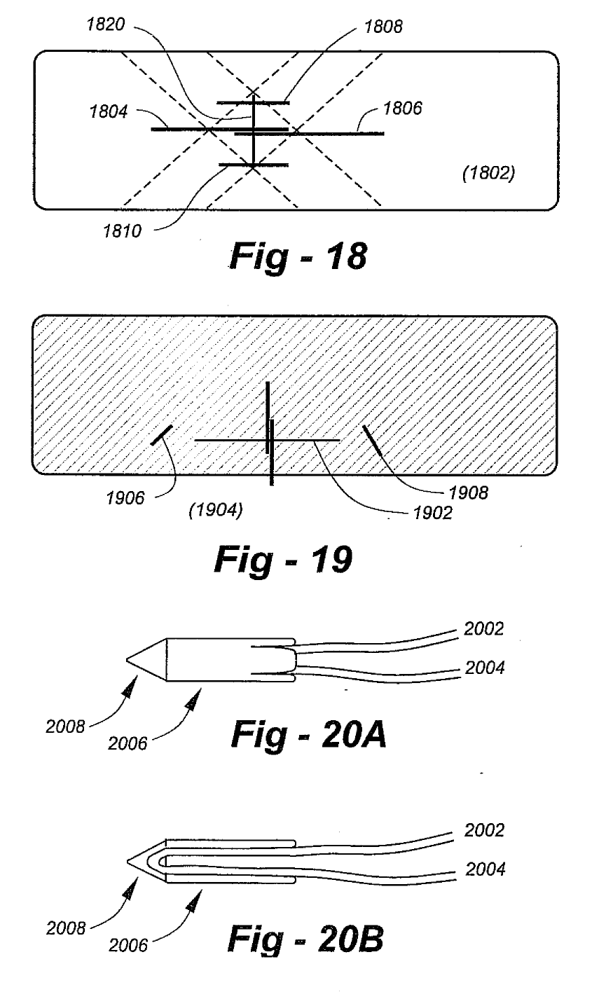

FIG. 18 is a posterior view of the IVD 1802 and the welded, flexible longitudinal fixation elements 1804, 1806, and two staple-like devices 1808, 1810 drawn in FIG. 17E. A vertical defect 1820 in the AF was drawn in the center of the IVD. The mesh patch and anti-adhesion cover were not drawn to better illustrate the fixation devices. Dotted lines were drawn to indicate the zones of injury. The fixation members were placed behind uninjured regions of the AF. The legs of the staple-like device extend behind the moderately injured regions of the AF.

FIG. 19 is a posterior view of an IVD with horizontal defect 1902 in the AF being drawn near the caudal portion of the IVD. A fixation member was placed in the vertebra 1904 caudal to the IVD and a fixation member was placed in the uninjured region of the AF cranial to the IVD. The fixation members, or anchors described in the text of other figures could be placed into holes drilled into the vertebrae. Staple-like fixation members 1906, 1908 were placed lateral to the defect in the AF. Staple-like fixation members placed in such locations are used to fasten the anti-adhesion cover and the mesh patch to the AF but not to close defects in the AF.

In FIG. 20A, the ends of two flexible longitudinal fixation elements 2002, 2004 extend from an anchor 2006 according to the invention. The tip 2008 of the anchor is tapered and may be forced through the AF without the beveled component of the insertion tool,

FIG. 20B is a longitudinal cross section of the embodiment of the invention drawn in FIG. 20A. The middle of the flexible longitudinal fixation element is embedded or otherwise fastened to the tip of the anchor. The end flexible longitudinal fixation element preferably does not slide through the tip of the anchor. Alternatively, the flexible longitudinal fixation element may slide through the tip of the anchor. For example, the flexible longitudinal fixation element could loop around an axle across the tip of the anchor.

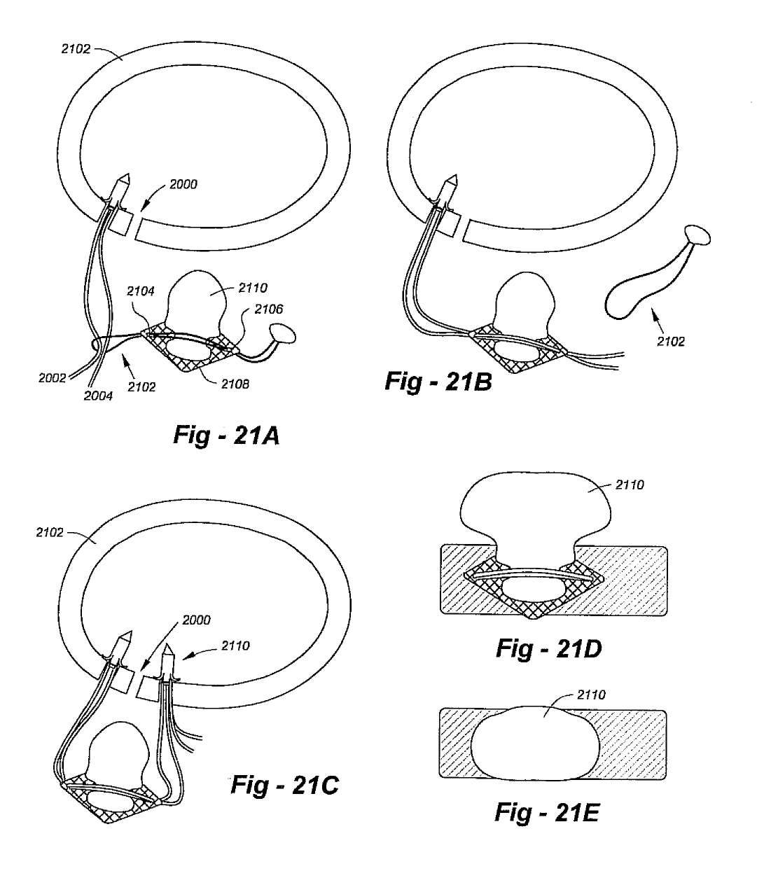

FIG. 21A is an axial cross section of an IVD and the embodiment of the invention drawn in FIG. 20A illustrating how the fixation member was inserted behind the inner layer of the AF 2102. The proximal ends of the flexible longitudinal fixation elements 2002, 2004 were passed through a suture passing device 2102. The suture passing device was threaded through holes 2104, 2106 in a mesh patch 2108. An anti-adhesion cover 2110 was fastened to the central portion of the mesh patch. For example the mesh patch and anti-adhesion cover could be glued together with a biocompatible glue, such as cyanoacrylate adhesive, welded or otherwise fastened together.

FIG. 21B is an axial cross section of an IVD and the embodiment of the invention drawn in FIG. 21A. The suture passer 2102 was used to pull the proximal ends of the flexible longitudinal fixation elements through the holes in the mesh patch.

FIG. 21C is an axial cross section of an IVD and the embodiment of the invention drawn in FIG. 21B. The proximal ends of the flexible longitudinal fixation elements were passed through a locking mechanism in a second fixation member 2110. The second fixation member was inserted behind the inner layer of the AF 2102 on the other side of defect 2000. The proximal ends of the flexible longitudinal fixation elements may be pulled to force the mesh patch against the AF and to pull the AF tissue on sides of the AF defect together.

The locking mechanism and the flexible longitudinal fixation elements preferably enable application of 10 to 40 pounds of tension on the flexible longitudinal fixation elements. Alternatively, 7, 8, 9, 41, 42, 43, less than 7, or more than 43 pounds of tension could be applied to the flexible longitudinal fixation elements. The proximal ends of the flexible longitudinal fixation elements are cut near the AF after the final tightening of such elements. A single flexible longitudinal fixation element may be used in an alternative embodiment of the invention.

FIG. 21D is a posterior view of an IVD and the embodiment of the invention drawn in FIG. 21C. FIG. 21E is a posterior view of an IVD and the embodiment of the invention drawn in FIG. 21D. The anti-adhesion cover 2110 was folded over the mesh patch and the flexible longitudinal fixation elements. Additional fixation elements are not required in the cranial and caudal portions of the device. Alternatively, fibrin glue (Tisseal, Baxter), platelet rich plasma, cyanoacrylate, staples, tacks, or other fixation material or devices may be used to attache the corners of the mesh patch to the AF.

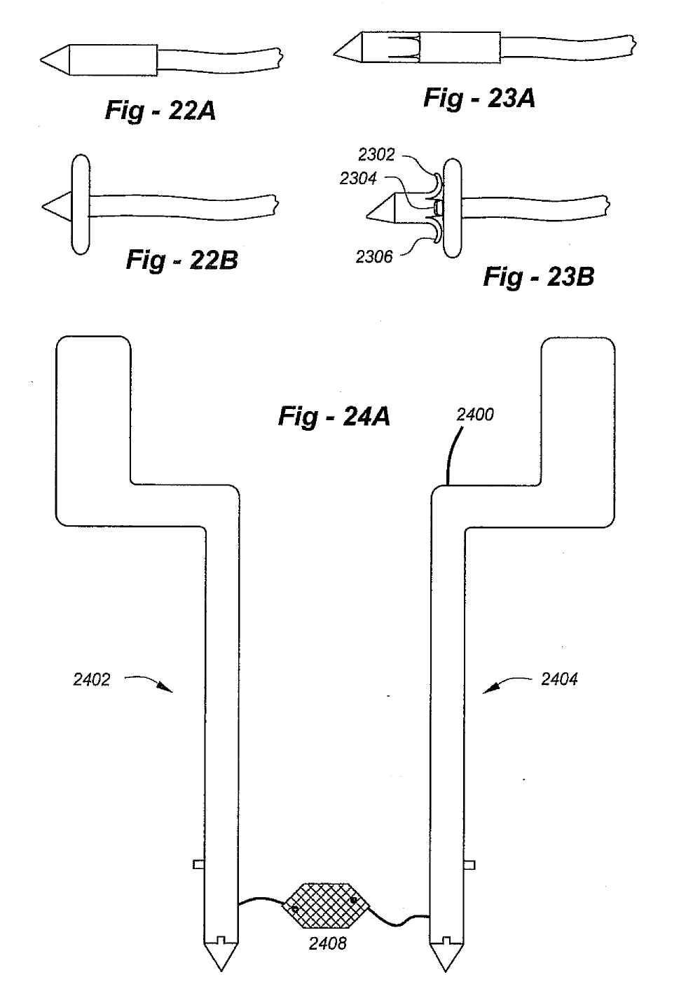

FIG. 22A is a lateral view of an alternative anchor according to the invention.

FIG. 22B is a lateral view of the embodiment of the invention drawn in FIG. 22A. The anchor component proximal to the tip was expanded in a radial direction relative to the shape drawn in FIG. 22A. The anchor component may expand in a radial direction secondary to a change in temperature, by releasing the device from the lumen of the bevel component of an insertion tool, by pulling on the proximal end of the flexible longitudinal fixation element while applying pressure on the proximal end of the radially expanding component with the distal end of the pusher component of the insertion tool.

FIG. 23A is a lateral view of a further alternative anchor wherein components 2302, 2304, 2306 were expanded in a radial direction relative to the shapes drawn in FIG. 23A. The anchor components are expanded after insertion of the anchor behind the outer layer of AF. The proximal radially expanded component may protect the AF from injury from the arms of the distal radially expanded component.

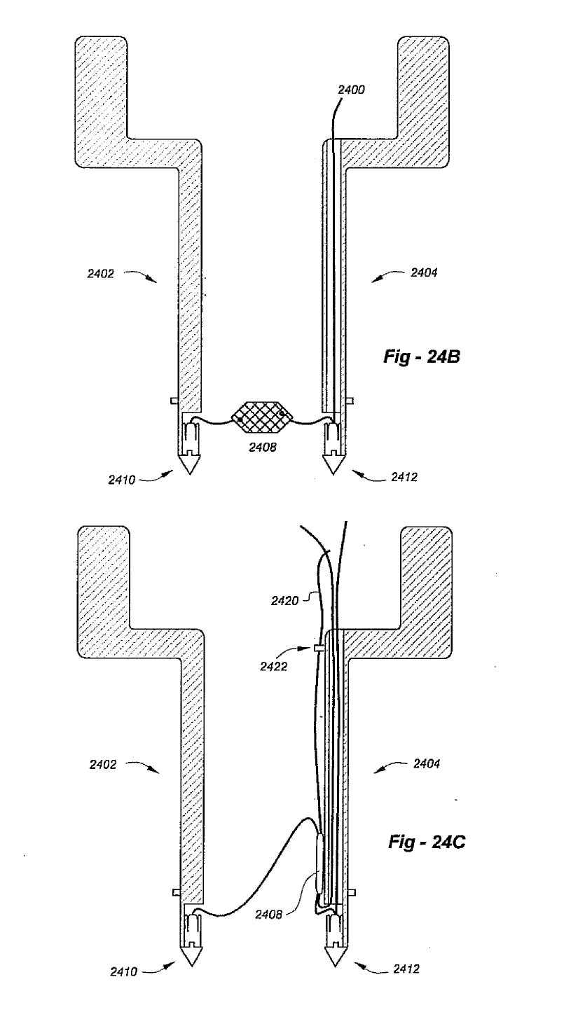

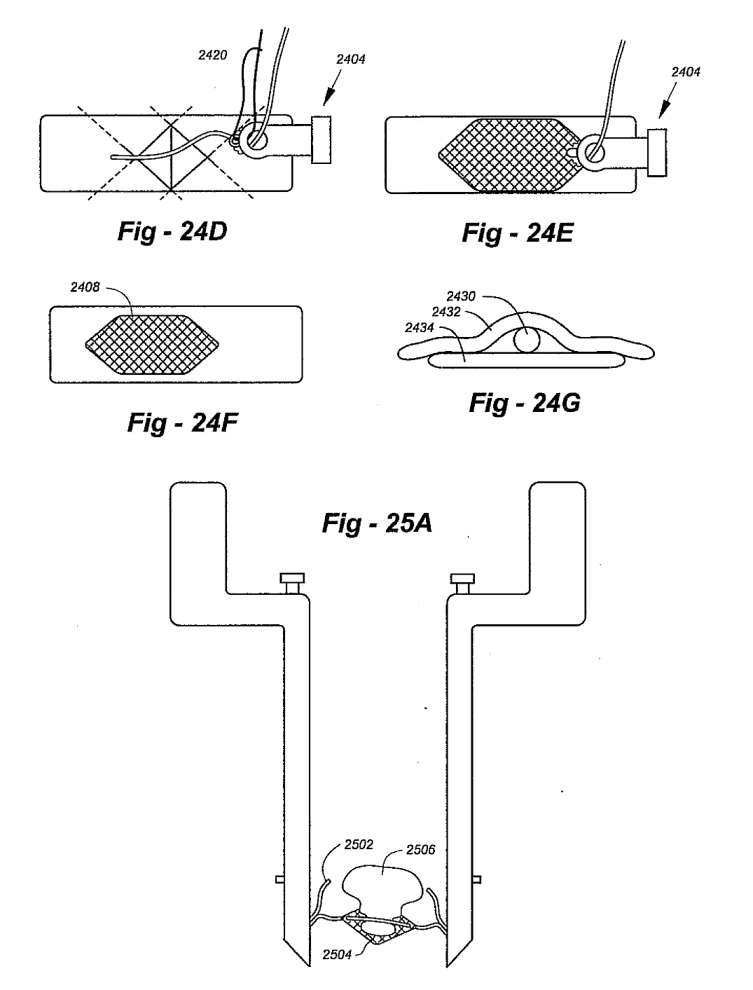

In FIG. 24A, a flexible longitudinal fixation element 2400 was passed through fixation members at the ends of two insertion tools 2402, 2404 and between a mesh patch 2406 and anti-adhesion cover 2408. The cranial and caudal ends of the mesh patch and anti-adhesion cover were fastened together. The flexible longitudinal fixation element slides freely between the central portions of the mesh patch and anti-adhesion cover. The device is preferably assembled by the manufacturer and supplied to surgeons in various sizes. For example, a small size may include a mesh patch that is 6 by 6 millimeters, a medium size may include a mesh patch 8 by 8 millimeters. and a large size may include a mesh patch 8 by 10 millimeters. The edges of the anti-adhesion cover preferably extend 2-3 millimeters or more beyond the edges of the mesh patch. Alternative size devices may be supplied including mesh patches that are 4, 5, 6, 7, 8, 9, 10, 11, 12, 13, or more millimeters tall by 4, 5, 6, 7, 8, 9, 10, 11, 12, 13, 14, or more millimeters wide.

FIG. 24B is a longitudinal cross section of the insertion tools and a lateral view of the fixation members and composite patch drawn in FIG. 24A. The distal end of the fixation member is fastened to a first fixation member and passes through an opening in the side of the insertion tool. The insertion tool is used to push the fixation members 2410, 2412, or anchors, through the AF. The fixation members have a projection that seats into an opening in the insertion tool. Rotation of the insertion tool also rotates the fixation member. The proximal end of the flexible fixation member passes between the mesh patch and anti-adhesive cover, into an opening in the insertion tool, through a locking mechanism in the second fixation member, and through the lumen of the second insertion tool.

In FIG. 24C, a second flexible longitudinal member 2420, such as a suture, was passed through openings at the ends of the anti-adhesion cover and mesh patch. The first end of the second flexible longitudinal member passes through an opening in a projection 2422 outside the insertion tool. The second end of the second flexible longitudinal element passes through the lumen of the insertion tool and through a loop in the first end of the second flexible longitudinal element. The second flexible longitudinal element is used to hold the mesh patch and anti-adhesion cover against the second insertion. Such invention prevents the anti-adhesion cover and mesh patch from obstructing the surgeon's view of the IVD.

FIG. 24D is a posterior view of an IVD and the embodiment of the invention drawn in FIG. 24C. The first fixation member was placed behind the inner layers of the AF in the intact AF zone lateral to the severely injured AF zone. The first insertion tool was removed after inserting the first fixation member. The second insertion tool 2404 is shown during placement of the second fixation member. The anti-adhesion cover and mesh patch can be seen against the side of the shaft of the insertion tool.

FIG. 24E is a posterior view of an IVD and the embodiment of the invention drawn in FIG. 24D. The mesh patch and anti-adhesion cover were released by removing the second flexible longitudinal member 2420. The patch was moved over the severely injured AF zone. Tension on the proximal end of the flexible longitudinal fixation element, tightens the flexible longitudinal fixation element, narrows the defect in the AF, and forces the mesh patch against the AS. One edge of the anti-adhesion cover is lies over the side of the shaft of the insertion tool.

FIG. 24F is a posterior view of the AF and the embodiment of the invention drawn in FIG. 24E. Excess flexible longitudinal fixation element, proximal to the locking mechanism in the fixation member, was cut and removed. The second insertion tool was removed, allowing the anti-adhesion cover to completely cover the flexible longitudinal fixation element and the mesh patch.

FIG. 24G shows how the edges of the mesh patch may be fastened to the anti-adhesion cover. A flexible longitudinal fixation element 2430 is seen between the anti-adhesion cover 2432 and mesh patch 2434. The materials and possible methods to fasten the materials were described in previous embodiments of the invention. Alternative embodiments could use two or more flexible longitudinal elements or locking mechanisms in all fixation members. Such configuration enables tightening the flexible longitudinal fixation elements both pulling on both ends of the flexible longitudinal elements. Alternative embodiments of the invention could use flexible longitudinal fixation elements with cross sectional shapes other than circular. For example cross sectional shapes of alternative flexible longitudinal fixation elements could be rectangular, oval, or other shape.

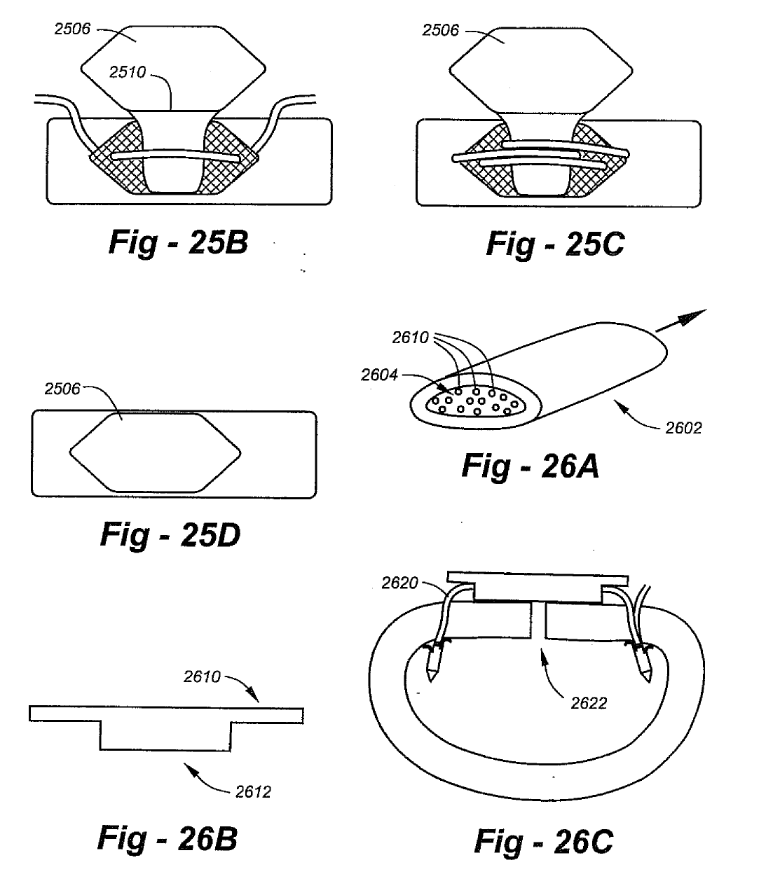

FIG. 25A is a lateral view of an alternative embodiment of the invention drawn in FIG. 24A. The ends of the flexible longitudinal fixation elements pass through openings or around axle-like members in the fixation members. The flexible longitudinal fixation element 2502 also passes through a mesh patch 2504 and over a portion of an anti-adhesion cover 2506. The mesh patch is designed to cover and reinforce the central portion of the severely injured region of the AF. For example, the mesh patch may cover the central 50-80 percent of the severely injured region of the AF. Alternatively, the mesh patch may cover the central 30, 35, 40, 45, 85, 90, or 95 percent of the severely injured portion of the AF.

FIG. 25B is a posterior view of an IVD and the embodiment of the invention drawn in FIG. 25A. Fixation members were pushed through the beveled component of the insertion tool and through AF tissue adjacent to a defective region of the AF. A fold 2510 can be seen in the anti-adhesion cover 2506.

FIG. 25C is posterior view of an IVD and the embodiment of the invention drawn in FIG. 25B. A welding tool was used to apply tension to the ends of the flexible longitudinal fixation element, weld the ends of the flexible element together, and cut the flexible element lateral to the weld. Alternatively, the ends of the flexible longitudinal fixation element could be fastened together by crimping, welding, or melting a third component to the ends of the flexible member, tightening a loosely tied knot, or by an alternative fastening method. FIG. 25D is a posterior view of an IVD and the embodiment of the invention drawn in FIG. 25C. The anti-adhesion cover 2506 was folded over the flexible longitudinal fixation element and the mesh patch.

FIG. 26A is an oblique view of an alternative embodiment of the mesh patch and anti-adhesion cover drawn in FIG. 24G and FIG. 6A of my co-pending patent application U.S. application Ser. No. 11/811,751 entitled “Devices for Herniation Repair and Methods of Use.” (Please add this application to the “Related U.S. application Data” section). The device 2602 is preferably made of an anti-adhesion material such as ePTFE. A lumen 2604 courses through the device in a left to right direction. The anterior wall of the device (the portion of the device that contacts the posterior AF) is perforated with closely spaced holes 2610 preferably 1 to 2 millimeters in diameter. Alternatively the holes may be 0.5, 0.6, 0.7, 0.8, 0.9, 2.1, 2.2, 2.3, 2.4, less than 0.5, or more than 2.4 millimeters in diameter. Alternatively, the holes may be square, triangular, hexagonal, or other shape in alternative embodiments of the invention. The centers of the holes are preferably spaced 2 to 4 millimeters apart. Alternatively, the holes may be spaced 1.5, 1.6. 1.7, 1.8, 1.9, 4.1, 4.2, 4.3, 4.4, less than 1.5, or more the 4.4 millimeters apart. The walls of the device are preferably 0.2 to 1.0 millimeters thick. Alternatively, the walls may be 0.05, 0.1, 1.1, 1.2, 1.3, 1.4, or more than 1.4 millimeters thick.

The device 2602 is preferably similar in length and width to the anti-adhesion components described the text of previous embodiments of the invention. For example, the device is preferably 4 to 40 millimeters wide and 6 to 20 millimeters long. Alternatively, the device may be 2, 3, 41, 42, 43, 44, or wider or 4, 5, 21, 22, 23, 24, 25, or more millimeters long. Embodiments of the device designed to cover most of the posterior portion of the AF may be 40 to 60 millimeters wide or wider. The device is designed to promote tissue ingrowth through the pores of the anterior wall of the device yet prevent adhesions between the posterior wall to the device and the nerves or dura that lie over the posterior wall of the device. Flexible longitudinal fixation elements pass through the lumen of the device.

In FIG. 26B, the posterior wall 2610 of the device is longer than the anterior wall 2612 of the device. The posterior wall of the device is preferably 4 to 20 millimeters longer than the anterior wall. Alternatively, the posterior wall may be 1, 2, 3, 21, 22, 23, 24, or more than 24 millimeters longer than the anterior wall of the device. The invention enables the posterior wall of the device to cover the flexible longitudinal fixation elements that extend slightly beyond the edges of the anterior wall of the device.

FIG. 26C is an axial cross section of an IVD and the embodiments of the invention drawn in FIGS. 11J and 26B. The flexible longitudinal fixation element 2620 courses through the lumen of the patch. The patch lies over an aperture, including a fissure 2622, in the IVD.

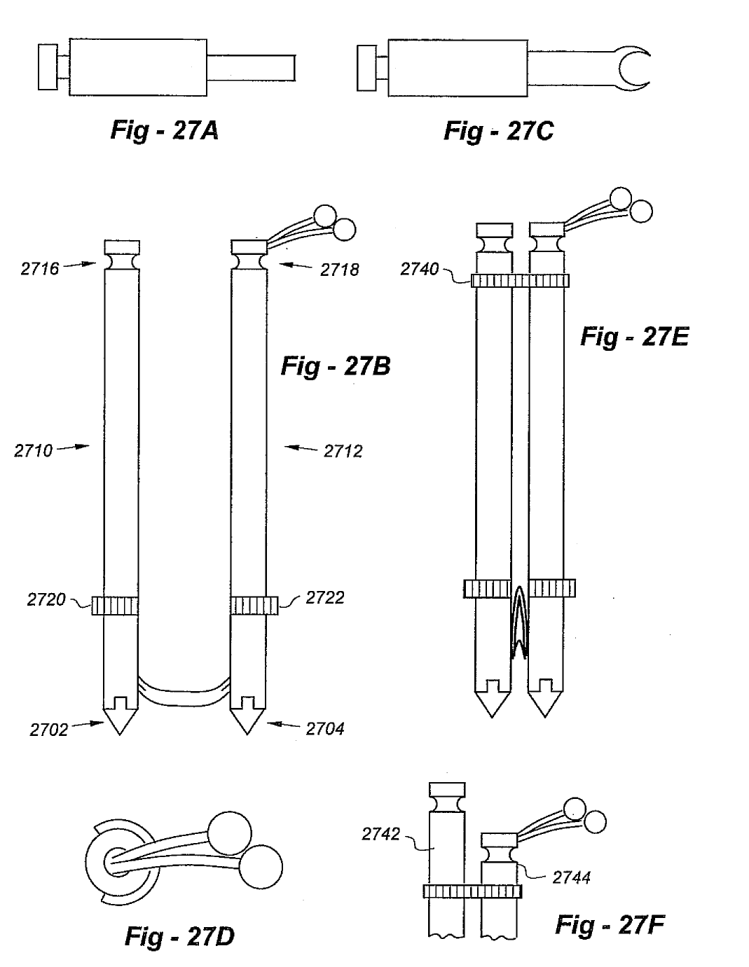

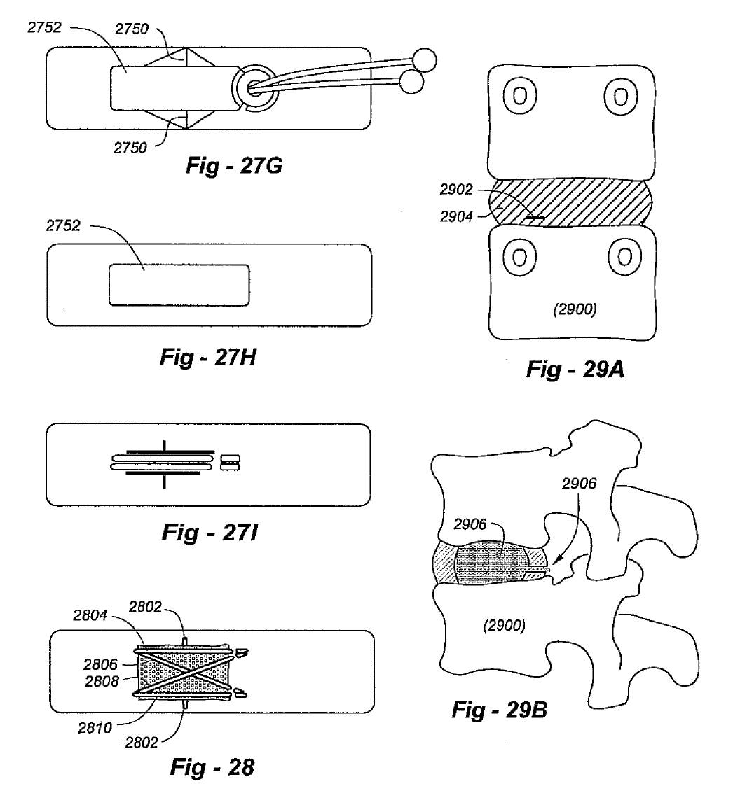

FIG. 27A is a lateral view of a releasable handle according to the invention. FIG. 27B is a lateral view of an alternative embodiment of the invention drawn in FIG. 24A. Fixation members 2702, 2704 can be seen at the distal ends of two insertion tools 2710, 2712. The handle drawn in FIG. 27A may be releasably attached to the recessed areas 2716, 2718 of either insertion tool. The insertion tools have depth stops 2720, 2722 to assure help surgeons determine how deep to insert the fixation members. The proximal ends of two flexible longitudinal fixation elements extend from the fixation member in the insertion tool on the left of the drawing, through the lumen of the lumen of the patch member drawn in FIG. 26B, through the fixation member in the insertion tool on the right of the drawing, and through the lumen of the insertion tool on the right of the drawing.

FIG. 27C is a view of the top of the embodiment of the invention drawn in FIG. 27A. The handle fits over the proximal end of the insertion tool. A spring loaded locking mechanism deploys a component the locks the handle to the insertion tool. The locking mechanism is released by pushing the button on the end of the handle.

FIG. 27D is a top view of the insertion tool drawn in FIG. 27B. The proximal ends of the flexible longitudinal fixation elements pass through a slot on the side of the insertion tool. The configuration enables a mallet to strike the proximal end of the insertion tool without damaging the flexible longitudinal fixation elements.

FIG. 27E is a lateral view of the embodiment of the invention drawn in FIG. 27B. The insertion tools were connected with a removable strap 2740. Alternative devices or mechanisms may be used to connect the insertion tools. For example, the insertion tools can be connected with Velcro, adhesive tape, paper tape, magnets, a plastic component or other device. The patch member is folded between the insertion tools. The assembled device is preferably 1.5 to 2.5 millimeters thick, by 5 to 8 millimeters wide, by 15 to 30 centimeters long. Alternatively, the assembled device may by 1.0, 1.1, 1.2, 1.3, 1.4, 2.6, 2.7, 2.8, 2.9, less than 1.0, or more than 2.9 millimeters thick, 4.5, 4.6, 4.7, 4.8, 4.9, 8.1, 8.2, 8.3, 8.4, 8.5, less than 4,5, or more than 8.5 millimeters wide, by 12, 13, 14, 31, 32, 33, 34, 35, less than 12, or more than 35 centimeters long.

FIG. 27F is a lateral view of the top of an alternative embodiment of the insertion tools drawn in FIG. 27E. The shafts 2742, 2744 of the tools are different lengths. The configuration allows surgeons to easily strike the top of one insertion tool. The anchor extending from the insertion tool on the left of the drawing is preferably inserted into the IVD or vertebra before the anchor extending from the insertion tool on the right of the drawing is inserted into the IVD or vertebra. The anchors may be inserted with aid of a spring loaded or pneumatic driven tool that exerts a rapid impacting force on the proximal ends of the anchors or shafts of the insertion tools. Other tools may be used to rapidly force the anchors through the AF. Rapid insertion of the anchors decreases the risk of bleeding from the epidural veins and heats the anchors, which speeds the shape change of temperature sensitive devices.

FIG. 27G is a posterior view of an IVD and the top of the embodiment of the invention drawn in FIG. 27F. A vertical defect 2750 can be seen in the center of the IVD. The outline of the severe injury zone extends from the vertical AF defect. The first fixation member was inserted behind the AF to the left of the vertical defect. The insertion tool was removed after inserting the fixation member. The top of the patch 2752 covers the flexible longitudinal fixation elements extending from the fixation member inserted on the left side of the AF defect. The top of a second insertion tool is seen on the right of the AF defect. The top of the mesh patch is folded against the insertion tool. The second fixation member is inserted by striking the top of the insertion tool with a mallet until the depth stop on the insertion tool is level with the AF.

FIG. 27H is a posterior view of an IVD and the embodiment of the invention drawn in FIG. 27G. The second insertion tool was removed after inserting the second fixation member, tightening the flexible longitudinal fixation elements, removing excessive flexible longitudinal fixation elements distal to the locking mechanism in the fixation member, and folding the top of the patch member 2752 over the ends of the flexible longitudinal fixation elements. The flexible longitudinal fixation elements are tightened by pulling on the proximal ends of the flexible longitudinal fixation elements or the enlargements at the ends of the flexible longitudinal fixation elements. Alternatively, the flexible longitudinal fixation elements may be tightened with tension limiting tool. For example, the proximal ends of the flexible longitudinal fixation elements could be inserted into the first portion of a tension limiting tool. The second end of the tension limiting tool could be placed against the top of the fixation member insertion tool. The tool assures the flexible longitudinal fixation elements are adequately tightened but not over tightened. For example, the tool could have a release mechanism that prevents more than 20-40 pounds of tension on the flexible longitudinal fixation elements. Alternatively, the tool could prevent more than 8, 9, 10, 11, 12, 13, 14, 15. 16, 17, 18, 19, 41, 42, 43,44, 45, less than 8, or less than 46 pounds of tension on the flexible longitudinal fixation elements.

FIG. 27I is a posterior view of the IVD and the embodiment of the invention drawn in FIG. 27H. The top of the patch member was removed to better illustrate the flexible longitudinal fixation elements 2760. One, three, four or more flexible longitudinal fixation elements may be used in alternative embodiments of the invention.

FIG. 28 is a posterior view of an IVD and an alternative embodiment of the invention drawn in FIG. 27I. The ends of a vertical defect 2802 in the AF are seen above and below the invention. Two fixation members were placed through the AF on the left side of the defect, and two fixation members were placed through the AF on the right side of the defect. Flexible longitudinal fixation elements 2804, 2806 pass from the fixation member in the upper left corner of the AF to the fixation members in the right side of the AF and flexible longitudinal fixation elements 2808, 2810 pass from the fixation member in the lower left corner of the AF to the fixation members in the right side of the AF. The flexible longitudinal fixation elements pass through the lumen in the patch member. The fixation members in the right side of the AF have the locking mechanism described in the text of FIG. 27H. Alternative configurations of the fixation members and flexible longitudinal fixation elements can be used to treat fissures or defects in the AF that are oriented in non-vertical directions. For example, two fixation members could be placed above and below a horizontal fissure in the AF. The flexible longitudinal fixation elements could be oriented ninety degrees relative to the flexible longitudinal fixation elements drawn in FIG. 28.

Alternatively, the flexible longitudinal fixation elements can be oriented 30,45,60,105,120,135,150,165, less than 30 or more than 165 degrees relative to the orientation of the flexible longitudinal fixation elements drawn in FIG. 28. As illustrated in FIG. 6B, one, two, three or more fixation members could be fastened to the vertebra.

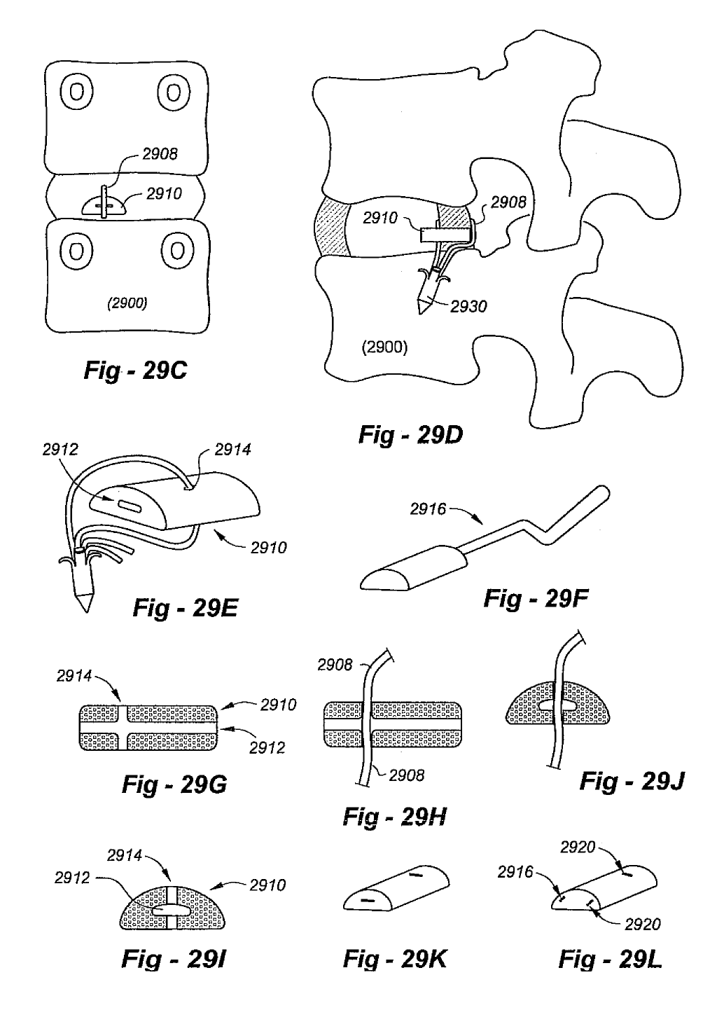

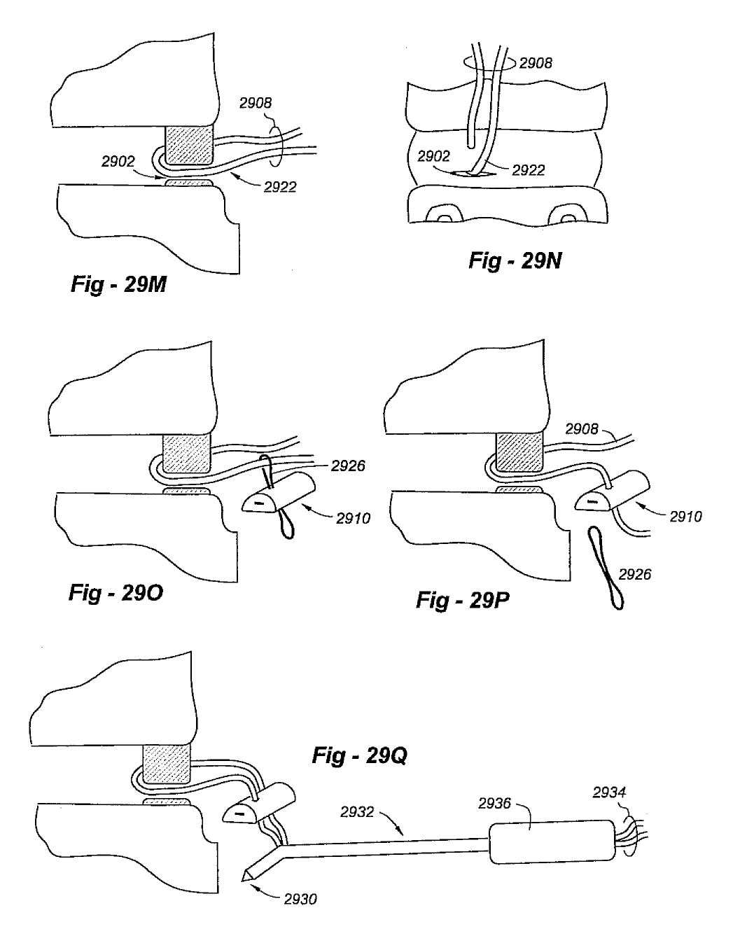

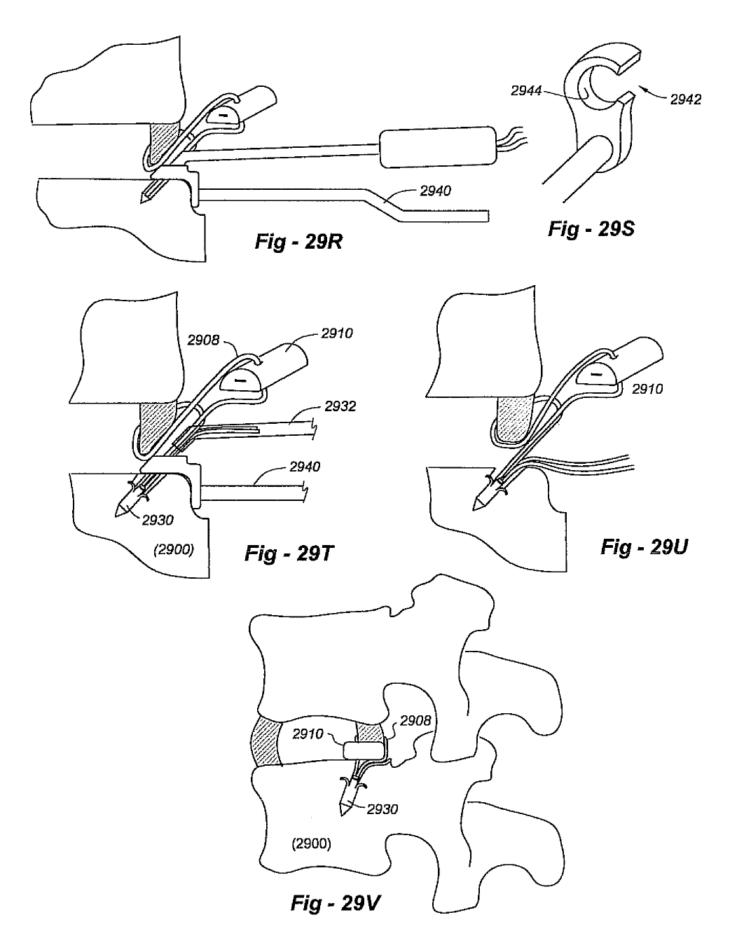

FIG. 29A is a posterior view of a coronal cross section of a portion of the spine. Similar to FIG. 6A, a transverse defect 2902 is seen near the caudal border of intervertebral disc (IVD) 2904. FIG. 29B is a lateral view of a partial sagittal cross section of the spinal segment drawn in FIG. 29B. Note that nucleus pulposis (NP) tissue 2906 extends into the defect in the AF.

FIG. 29C is a posterior view of a coronal cross section of a portion of the spine and an alternative embodiment of the invention drawn in FIG. 6B and FIG. 2B of my co-pending U.S. application Ser. No. 11/811,751 entitled “Devices for Herniation Repair and Methods of Use”. A transverse lumen or passageway passes through the intra-aperture component of the device 2910. The intra-aperture component was placed in an aperture or defective region of the AF. The superior arm 2908 of the flexible longitudinal fixation component of the device lies over the proximal end of the intra-aperture component.

FIG. 29D is a lateral view of a partial cross section of the spinal segment and embodiment of the invention drawn in FIG. 29C. The superior arm of the flexible longitudinal fixation component 2908 passes through the AF adjacent to the aperture in the AF and through a fixation member 2910 that is placed into the vertebra. The distal arm of the flexible longitudinal component also passes through the vertebral fixation member. The central portion of the flexible longitudinal component lies within a vertical passageway through the intra-aperture component. The fixation member 2910 is placed into the lower vertebra in this case since the defect is proximate to the superior endplate of that vertebral body. The invention is not limited in this regard, however, in that a flexible longitudinal fixation component may be anchored to an upper vertebral body or both upper and lower body if the situation so warrants.

The intra-aperture component 2910 preferably slides along the flexible longitudinal fixation component. Alternatively, the intra-aperture component may be fastened to the flexible longitudinal fixation component in a manner that prohibits sliding of the intra-aperture component over the flexible longitudinal component. For example, the two components could be fastened together with an adhesive.

The flexible longitudinal fixation component is preferably made of high tensile strength multi-filament or braided polyester. For example, the flexible longitudinal fixation component could be made of #2 to #5 sized Fiberwire (Arthrex, Naples, Fla., USA), Orthocord (DePuy Orthopaedics, Warsaw, Ind., USA), sutures from Tornier (Edina, Minn., USA), nylon or other type or size suture material.

The vertebral fixation member is preferably made of shape memory material such as Nitinol and contains a cinch-like mechanism that allows the arms of the flexible longitudinal fixation component to slide more easily in one direction than another direction. The locking mechanism permits tightening of the arms the flexible longitudinal fixation component and locking of the arms of the flexible longitudinal fixation component in the tightened position.

Arms from the proximal end of the vertebral fixation component expand or move from the central axis of the vertebral fixation component into vertebral bone after the vertebral fixation component is impacted into the vertebra. The expanded shape of the vertebral fixation component resists forces that attempt to expulse the vertebral fixation component from the vertebra. The vertebral fixation component is preferably 2 to 8 millimeters in diameter and 5 to 15 millimeters long. Alternative sizes of the vertebral fixation component may be used in other embodiments of the invention.

The vertebral fixation component is preferably impacted into predrilled holes in the vertebra. Alternatively, the vertebral fixation component may be impacted into the vertebra without a predrilled hole in the vertebra. The vertebral fixation component preferably incorporates the expansion and locking mechanism used in the Sapphire suture anchor (Tornier, Edina, Minn., USA). Anti-backout and suture locking mechanisms of alternative suture anchors, or suture anchors could be used in alternative embodiments of the invention. The intra-aperture component of the invention covers the hole created in the vertebra during placement of the vertebral fixation component. The intra-aperture component prevents migration of NP tissue into the hole in the vertebra.

We discovered migration of NP tissue into 1.5 mm diameter×8 mm long holes drilled through apertures in the AF and through vertebral endplates (VEPs) of IVDs of twenty sheep, in the manner illustrated in the drawing, which contributed to disc degeneration in these animals. Certain aspects of the invention seek to prevent disc degeneration by preventing NP migration into the vertebrae following surgical repair of the spine, especially the surgical repair of apertures in the AF.

The flexible longitudinal fixation component and the vertebral fixation component could be used in a similar method without the intra-aperture component in alternative embodiments of the invention. In such cases, the flexible longitudinal fixation component pulls native AF over the hole used for insertion of the vertebral fixation component. This alternative embodiment of the invention could be used for small apertures in the AF. Alternatively, two or more devices with two or more vertebral fixation, flexible longitudinal fixation, and intra-aperture components could be used in alternative embodiments of the invention.

FIG. 29E is an oblique view of the intra-aperture component 2910 drawn in FIG. 29C. The device has transverse and vertical passageways 2912, 2914. The vertical passageway 2914 is preferably 0.4 to 1.5 mm in diameter. Alternatively, the diameter of the vertical passageway in the intra-aperture component could be 0.2, 0.3, 1.6, 1.7, less than 0.2 or more than 1.7 mm in diameter.

The flexible longitudinal fixation component, preferably with a diameter the same size or slightly smaller than the diameter of the vertical passageway through the intra-aperture component, is passed through the passageway in the intra-aperture component to hold the device in the aperture in AF and hold the device over the hole in the vertebra. For example, high strength multifilament polyester suture material with a tensile strength of more than 100 pounds and a diameter of 0.7 mm could be passed through a 0.7 mm diameter vertical passageway through the intra-aperture component. A tight fit between the flexible longitudinal fixation component and the vertical passageway in the intra-aperture component prevents NP tissue migration along the flexible longitudinal fixation component and into the hole in the vertebra. The flexible longitudinal fixation component is preferably 30 to 60 centimeters long, but could be shorter or longer in alternative embodiments of the invention.

The transverse passageway 2912 in the intra-aperture component 2910 is preferably 1.1 to 2.0 mm in diameter. Alternatively, the diameter of the transverse passageway through the intra-aperture component could be 1.07, 1.08, 1.09, 2.01, 2.02, less than 1.07 or more than 2.02 mm in diameter in other embodiments of the invention. Transverse passageways preferably pass directly through the intra-aperture without openings in the sides of passageway that are as large or nearly as the diameter, width, or height of the passageway. Such side openings, particularly side openings without outlets, permit NP tissue accumulation in the intra-aperture component. Accumulation of NP tissue within the intra-aperture component may obstruct adjacent passageways and prevent extrusion of NP tissue around the intra-aperture component.

According to the invention, the direct passageways in the intra-aperture component are designed to minimize accumulation NP tissue within the device, facilitate NP extrusion into, through, and from the intra-aperture component. Such passageways in the intra-aperture component diminish peak intradiscal pressures while connective tissue grows into the device, thus providing long-term fixation to the AF and the vertebrae. The diameters of cylindrical direct transverse passageways or the widths or heights of non-cylindrical direct transverse passageways could be as small as 0.5, 0.6, 0.7, 0.8, 0.9, 1.0 millimeters or smaller in alternative embodiments of the invention.

As described in my co-pending application U.S. Ser. No. 11/811,751, transverse passageways through the intra-aperture component allow migration of NP tissue from the IVD through the device. Migration of NP tissue through the device prevents excessive pressure from the NP on the device. Intradiscal pressure often exceeds 330 PSI. Intra-aperture devices that seal the AF, thus preventing the escape of NP tissue through apertures in the AF, are exposed to such high pressure by the NP and are at risk of catastrophic failure. Such devices are analogous to a cork in a bottle of champagne. Just as such corks are ejected from champagne bottles by high pressure inside the bottle, intra-aperture devices that seal the IVD will likely be ejected from the AF and into the nerves in the spinal canal.

The passageway or passageways in the intra-aperture component of the invention provide a pressure release mechanism to avoid migration of the device, thus protecting the nerves in the spinal canal. We applied axial compression to human cadaver spines previously repaired mesh devices placed over apertures in twenty-nine IVDs. The mesh devices were loosely fastened to the spine and could be pulled from the spine with forces as low 63.3N. We discovered mesh devices that did not seal the IVD and thus allowed NP material to around the device prevented device migration of all twenty-nine devices despite applying axial loads, as high as 5598 N, which fractured vertebrae in thirteen specimens. These experiments showed NP tissue extrusion around devices that do not seal apertures in the IVD, even devices with relatively poor fixation, thereby preventing expulsion of the device. The findings confirmed that allowing extrusion of NP tissue in or around intra-anular components which do not seal apertures in IVDs is effective in preventing expulsion of the device.

We measured intra-discal pressures in a different experiment. Axial load was applied to human cadaver spines previously repaired with mesh devices loosely applied over apertures of 13 IVDs. The mesh devices allowed NP tissue to extrude through apertures in the IVD. Intra-discal pressures of such unsealed repaired IVDs remained quite low (average less than 35 PSI range 4-67 PSI) despite applying an average compression load of 2377 N and as high as 5598 N, a load high enough to fracture three vertebra in the study. Wilke et al. (Spine 1999, 24(8):755-762) found in vivo intradiscal pressures of the native sealed IVD as high as 2.3 MPa (334 PSI) with such activities as lifting a 20 Kg package with the subject bent over with a round back posture. Such activities rarely produce sufficient load to fracture vertebra, as we did in our study. All 13 mesh devices were intact without migration at the end of the study. The experiment showed IVDs with unsealed apertures have substantially lower intradiscal pressures than sealed IVDs when subjected to similar axial loads. The study showed extrusion of NP tissue through unsealed apertures in the IVD prevented high intradiscal pressures and thus prevented expulsion or damage of the devices. These studies showed that intra-aperture components that allow migration of NP tissue through passageways in the component or around the component prevent high intradiscal pressure that leads to device expulsion or damage to the device.

The intra-aperture components according to this invention are preferably manufactured from allograft or xenograft AF tissue, fascia, dermis, tendon, ligament, bone, demineralized bone, or other tissue. However, allograft and xenograft tissues is preferably modified by placement of passageways that enable extrusion of NP tissue to be used in the manufacture of intra-aperture components taught in this invention. For example, AF tissue does not contain channels or passageways large enough to allow extrusion of NP tissue through the tissue. In fact, the AF of adults contains no blood vessels. Native AF tissue seals the IVD, prevents extrusion of NP tissue, and enables the high intradiscal pressure seen in humans. Cells such as mesenchymal stem cells can migrate from the vertebra, along the hole made in the vertebra to insert the vertebral fixation component and into intra-aperture components manufactured of tissue to revitalize such components.

Alternatively, the intra-aperture components can be made of polyester, polypropylene, polyurethane, or other synthetic biocompatible material. Intra-aperture components made of allograft AF tissue are preferably oriented with the fibers of the donor tissue oriented ninety degrees relative to the fibers of the recipient native AF to take advantage of the high tensile strength of the AF tissue in the plane that passes through rather than between the lamellae of the AF. Alternatively, allograft or xenograft AF intra-aperture components can be oriented with their fibers in the direction of the native AF.

Passageways are preferably machined in allograft tissue devices, and possibly synthetic devices, by passing appropriately sized tapered, rather than cutting, needles through the tissue. The intra-aperture component is preferably 3 to 15 millimeters wide, 1 to 10 millimeters tall and 4 to 15 millimeters long. Alternatively, the component could be less than 3 to 4 or more than 10 to 15 millimeters wide or long, respectively and less than 1 millimeter or more than 10 millimeters tall. The intra-aperture component is preferably hemi-cylindrical in shape. Alternatively, such component may have a cylindrical, cube, box, or other shape. Grooves in the direction of the transverse passageway could be cut into the surface of the intra-component to facilitate NP particle migration between the intra-aperture component and the AF.

FIG. 29F is an oblique view of a sizing tool 2916 that is placed into the aperture in the AF to select the best size intra-aperture component for the defect in the AF. Various sized intra-aperture components are preferably manufactured in the size ranges listed in the text of FIG. 29E. Such sizing tools help prevent surgeons from inserting intra-aperture components that are larger than the aperture. The invention seeks to insert intra-aperture components than are the same size or smaller than apertures in the AF and uses intra-aperture components that do not expand or that may contract after insertion into apertures in the AF, to facilitate migration of NP tissue.

FIG. 29G is a lateral view of a sagittal cross section of the intra-aperture component drawn in FIG. 29E. FIG. 29H is a lateral view of a sagittal cross section of the intra-aperture component and a portion of the flexible longitudinal fixation component drawn in FIG. 29G. The flexible longitudinal fixation component is seen within the vertical passageway through the intra-aperture component, similar to the configuration illustrated in FIG. 29D. FIG. 29I is a posterior view of a coronal cross section of the intra-aperture component drawn in FIG. 29E.

FIG. 29J is a posterior view of a coronal cross section of the embodiment of the invention drawn in FIG. 29H. The flexible longitudinal fixation component 2908 is seen within the vertical passageway 2914. The flexible longitudinal fixation component preferably bisects the transverse passageway leaving two smaller openings that are 1.1 mm or wider each. For example, the flexible longitudinal fixation component could be 0.8 mm wide and the transverse passageway 3 mm wide thus leaving two 1.1 mm wide openings in the transverse passageway. Tension on the arms of the flexible longitudinal fixation component following implantation of the device stiffens the flexible longitudinal fixation component and enables it to cut 3 mm wide pieces of NP tissue in the transverse passageway of the device into two narrower pieces of NP tissue.

The invention enables extrusion of NP particles as large as 1.1 mm directly through the intra-aperture component. Larger particles of NP tissue, which may be as large or larger than the diameter of the intra-aperture component are particulated into smaller NP particles by passage through the intra-aperture component. High intradiscal pressure pushes large particles of NP tissue are against the distal end of the intra-aperture component. The high intradiscal pressure than forces the portion of the large NP particle that lies over the opening of the transverse passageway on the distal side of the intra-aperture component, into the passageway of the component whereby such tissue is extruded from the intra-aperture component. Additional NP tissue from the large NP particle then flows into the passageway in the intra-aperture component, thus repeating the extrusion process, if the intradiscal pressure increases.

FIG. 29K is an oblique view of the intra-aperture component drawn in FIG. 29E. The openings of the passageways are preferably closed in the resting state of the component. Connective tissue from the AF preferably grows through the component and across the passageways to provide long-term fixation of the device to the spine. Expansion of the component in-situ following insertion in the aperture is preferably avoided. Such expansion of the component reduces the size of the space or potential space between the component and the AF, thus reducing NP tissue extrusion between the component and the AF, may reduce the diameter of the transverse passageway if the material of the device expands thus impeding NP tissue extrusion through the transverse passageway of the component, and may increase tension on the flexible longitudinal fixation component causing the flexible longitudinal fixation component to break or cut through the AF tissue allowing the device to migrate.

In situ expansion of the intra-aperture components can be avoided by supplying fully hydrated components and by avoiding constriction of the components during insertion into the AF. Intra-aperture components manufactured of tissue could be supplied in saline filled containers, soaked in saline before surgery, or frozen in a fully hydrated state to prevent the component from imbibing fluids, thus swelling, in-situ. Alternatively, tissue components may be soaked or stored in slightly hypotonic saline or other solution to cause swelling of the component. Such swollen intra-aperture components could shrink after placement in an aperture in the AF, thus providing space for NP tissue migration between the component and the AF,

FIG. 29L is an oblique view of an intra-aperture component having two transverse passageways 2916, 2918 that do not communicate with the vertical passageway 2920. Three, four or more transverse passageways may be used in alternative embodiments of the invention. The drawing also illustrates one of several alternative shapes of the component. The component is preferably made of the materials listed in FIGS. 29C-K, and is preferably similar in size to the intra-aperture components drawn in FIGS. 29C-K.

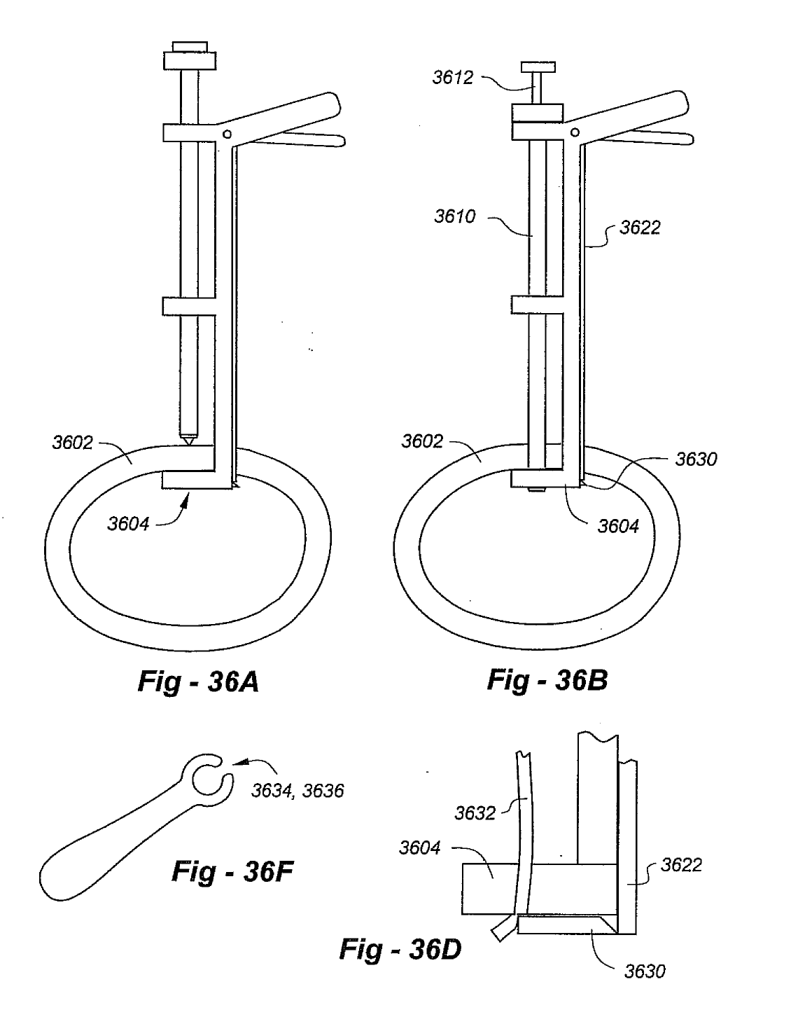

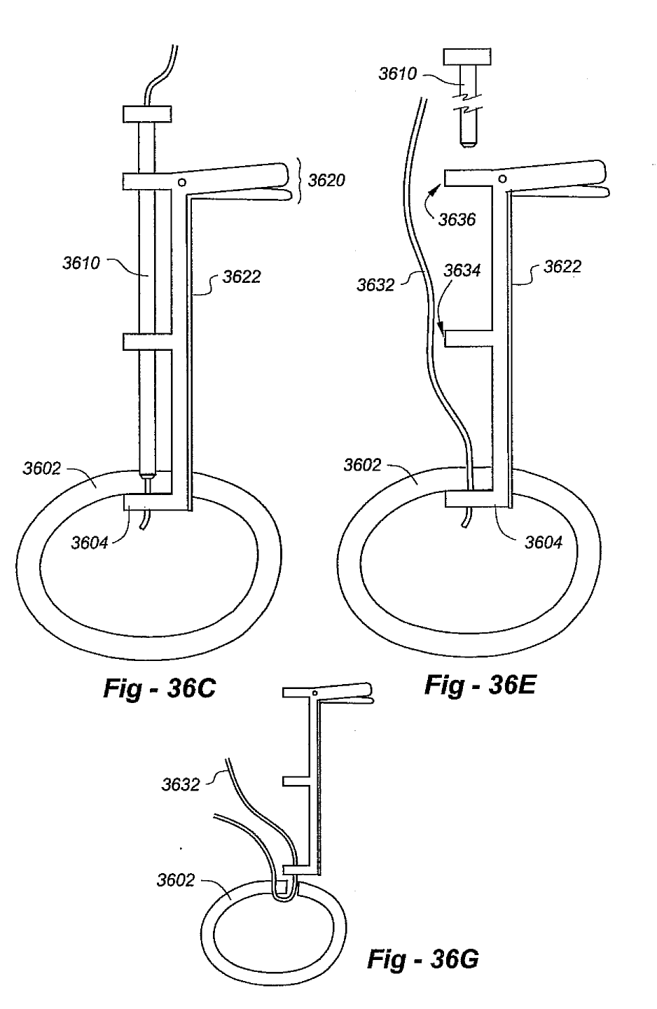

FIG. 29M is lateral view of a partial sagittal cross section of portion of the spine, a partial exploded view and the first step to insert the embodiment of the invention drawn in FIG. 29E. The inferior arm 2922 of the flexible longitudinal fixation component was passed through AF tissue adjacent to an aperture in the AF and through the aperture 2902. The flexible longitudinal fixation component could be placed in such location using the invention illustrated in FIGS. 36A-G. FIG. 29N is a posterior view of a partial coronal cross section of the portion of spinal segment and invention drawn in FIG. 29M.

FIG. 29O is lateral view of a partial sagittal cross section of the portion of the spine, a partial exploded view of and the second step to insert the embodiment of the invention drawn in FIG. 29E into the IVD. The first end, or inferior arm, of the flexible longitudinal fixation component was passed through a loop 2926 previously placed through the vertical passageway in the intra-aperture component 2910. The loop is preferably placed through the intra-aperture component during the manufacturing process.

FIG. 29P is a lateral view of a partial sagittal cross section of the portion of the spinal segment drawn in FIG. 29O, a partial exploded view and the third step to insert the embodiment of the invention drawn in FIG. 29E. The first end of the flexible longitudinal fixation component was passed through the vertical passageway in the intra-aperture component by pulling the loop through the intra-aperture component.

FIG. 29Q is a lateral view of a partial sagittal cross section of the portion of the spinal segment drawn in FIG. 29P, and the embodiment of the invention and the fourth step to insert the embodiment of the invention into the spine. The arms or ends of the flexible longitudinal fixation element were passed through the locking mechanism of the vertebral fixation component 2930. The vertebral fixation component was placed into an angled tool 2932 used to insert the component into the vertebra. The angle in the shaft of the tool is preferably between 10 and 30 degrees. Alternatively, such angle could be 8, 9, 31, 32, less than 8 or more than 32 degrees in other embodiments of the invention. The ends 2934 of the flexible longitudinal fixation component extend outside the handle 2936 of the cannulated instrument 2932.

FIG. 29R is a lateral view of a partial sagittal cross section of the portion of the spinal segment drawn in FIG. 29Q, a vertebral fixation component insertion guide 2940, and the fifth step to insert the embodiment of the invention drawn in FIG. 29E. The distal end of the guide 2940 is passed through the aperture in the AF and over the posterior corner of the cranial end of the vertebra caudal to the IVD. The guide preferably starts the vertebral fixation component at a point 3 to 8 millimeters anterior to the posterior wall of the vertebra, directs the component towards the anterior and inferior region of the vertebral body, prevents the component from slipping along the VEP as the component is impacted into the vertebra, and protects and retracts the AF tissue cranial to the aperture. Alternatively, the hole could be started 1, 2, 9, 10, less than 1 or more than 10 mm anterior to the posterior wall of the vertebra in alternative embodiments of the invention.

The vertebral fixation component may also be started in the posterior wall of the vertebra and directed at other angles relative to the vertebra in alternative embodiments of the invention. The vertebral fixation is preferably 3 to 5 millimeters in diameter and 5 to 15 millimeters in length. Alternative sizes of the vertebral fixation component could be used in alternative embodiments of the invention. The proximal end of the vertebral fixation component is preferably recessed 3 to 15 millimeters below the surface of the vertebra. Alternatively, the proximal end of the vertebral fixation component could be recessed closer to or further from the surface of the vertebra.

FIG. 29S is an oblique view of the distal end of the guide 2940 drawn in FIG. 29R. A longitudinal opening 2942 extends along the side of the cylindrical opening 2944 of the tool. The feature enables the guide to contain the shaft of the vertebral fixation component insertion tool and allows the flexible longitudinal fixation component to escape from the guide when the shaft of the vertebral fixation component insertion tool is pulled from the guide.

FIG. 29T is lateral view of a partial sagittal cross section of the portion of the spinal segment drawn in FIG. 29R and the sixth step to insert the embodiment of the invention drawn in FIG. 29E into the spine. The vertebral fixation component 2930 was impacted into vertebral body and the distal tip of the vertebral fixation component insertion tool 2932 was extracted from the guide. Vertebral fixation components could be inserted into predrilled holes in alternative embodiments of the invention.

FIG. 29U is a lateral view of a partial sagittal cross section of the portion of the spinal segment drawn in FIG. 29T and the seventh step to insert the embodiment of the invention drawn in FIG. 29E into the spine. The vertebral fixation component insertion tool and the guide were removed. The distal tip of the intra-aperture component 2910 is inserted into the aperture in the next step of the method followed by tension on the arms of the flexible longitudinal fixation component.

FIG. 29V is a lateral view of a partial sagittal cross section of the portion of the spinal segment drawn in FIG. 29U and the final position of the assembled invention drawn in FIG. 29U. The inter-aperture component 2910 covers the hole in the vertebra used to place anchor 2930, lies within the aperture, provides escape routes for NP tissue through and around the component, and is held in position by the VEP, AF, and a now closed loop of formed by the arms of the flexible longitudinal fixation component 2908 and vertebral fixation component 2930. The superior arm of the flexible longitudinal fixation component passes over the proximal end of the intra-aperture component, as perhaps best seen in FIGS. 29C and 29W. The configuration of the assembled device prevents migration or bulging of the intra-aperture component into the nerves of the spinal canal. The large surface area of the elongate, spaghetti-shaped, pieces of NP than can preferably extrude through or around the intra-aperture component facilitates resorption of the tissue. Flexible elongate extruded NP particles are also less likely to compress spinal nerves than large stiffer more spherical-shaped NP particles or extruded devices.

FIG. 29W is a posterior view of a partial coronal cross section of the spinal segment drawn and the embodiment of the invention drawn in FIG. 29V. Wider apertures through the AF could be repaired with wider intra-aperture components that two or more superior and inferior flexible longitudinal fixation arms and two or more vertebral fixation components. The assembled device could be fastened to the spine in an alternative embodiment of the invention. For example, the superior arm of the flexible longitudinal fixation component could be removed from the vertebral fixation component, passed through AF tissue adjacent to the aperture in the AF, followed by passing the superior arm of the flexible longitudinal fixation component back through the vertebral fixation component and the vertebral fixation component impacted in the vertebra. Tension on the ends of the arms of the flexible longitudinal fixation component advances the arms through the locking mechanism of the vertebral fixation component thus reducing the mobility of the intra-aperture component.

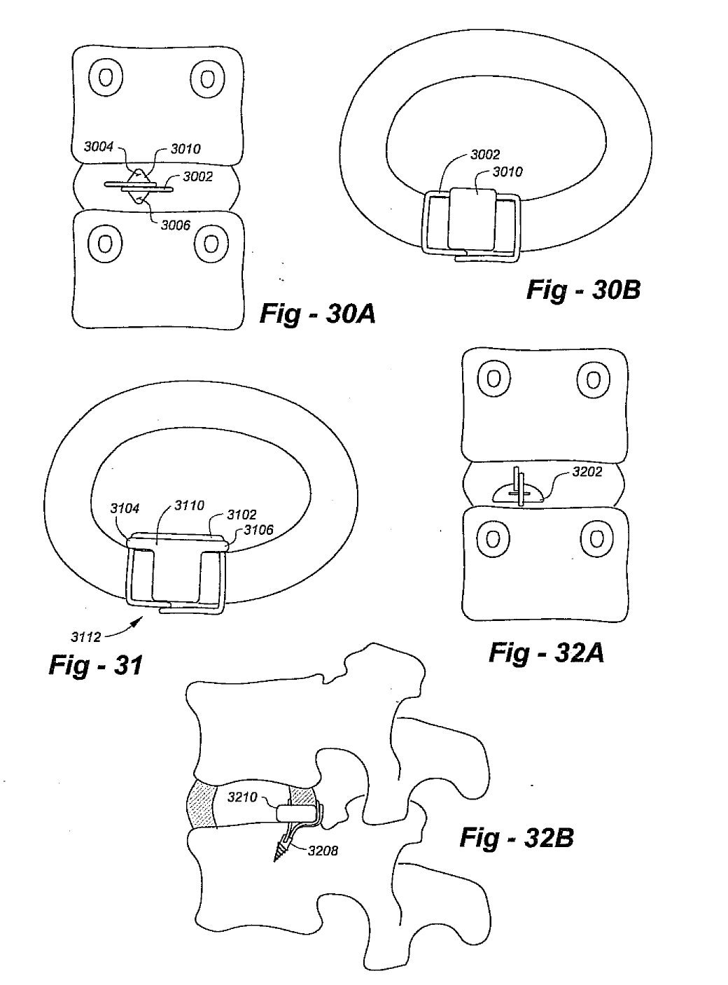

FIG. 30A is a posterior view of a partial coronal cross section of a spinal segment and an alternative embodiment of the invention. The ends of the left and right arms of a flexible longitudinal fixation component 3002 were welded together, for example, using the Axya welding system (Beverly, Mass., USA). The arms of the flexible longitudinal fixation component pass through AF tissue on either side of a vertical defect in the AF but do not pass through a vertebral fixation component. The embodiment of the invention is particularly suited for apertures in the AF that are not near either vertebra. Two transverse passageways 3004, 3006 are seen in the intra-aperture component 3010. The drawing illustrates the preferred shape of intra-aperture components that placed in apertures that are not adjacent to vertebrae. Alternative intra-aperture component shapes including the described in other embodiments of the invention could be used in alternative embodiments of the invention. The materials listed in the text of FIGS. 29C-W are preferably used in the embodiments of the invention taught in FIGS. 30A-48E. The sizes of the components listed in the text of FIGS. 29C-W are preferably used in the embodiments of the invention taught in FIGS. 30A-48E.

FIG. 30B is a partial transverse cross section of the IVD and embodiment of the invention drawn in FIG. 30A. The flexible longitudinal fixation component 3002 passes through a transverse passageway in the intra-aperture component. Such transverse passageway in the intra-aperture component is perpendicular to the transverse passageways that enable extrusion of NP tissue. The flexible longitudinal fixation could be fastened to the intra-aperture component with adhesive or other material or mechanism in alternative embodiments of the invention.

FIG. 31 is a partial transverse cross section of an IVD and an alternative embodiment of the invention, showing how the arms of the flexible longitudinal fixation component 3102 pass through enlarged distal ends 3104, 3106 of the intra-aperture component 3110. The device could be manufactured with the materials listed in the text of FIG. 29C-W and the components could be similar in size to the size of the components listed in FIGS. 29C-W. The ends of the arms of the flexible longitudinal fixation component were fastened together at 3112. Welding or other method or device could be used to fasten the arms of the flexible longitudinal fixation component together.

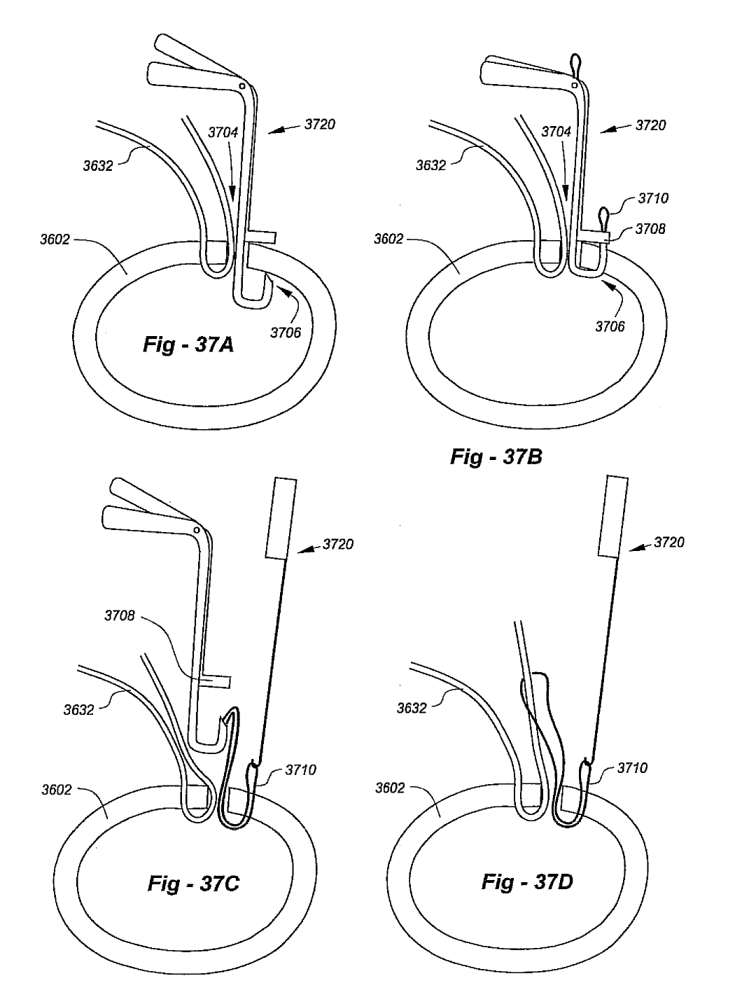

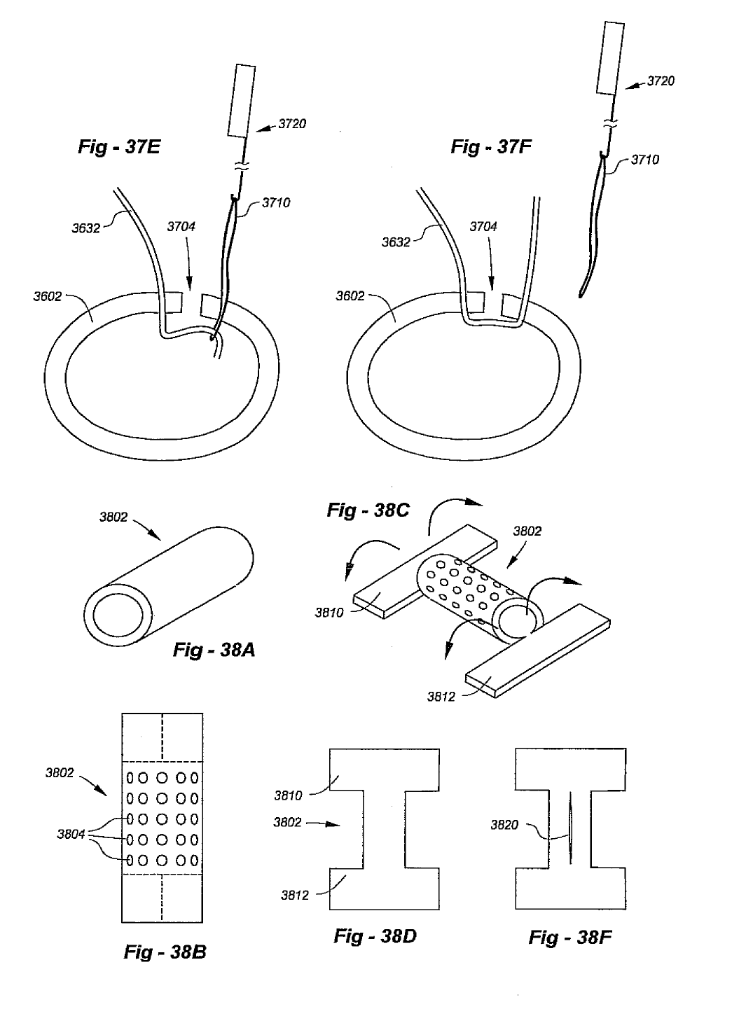

FIG. 32A is a posterior view of a partial coronal cross section through a spinal segment and an alternative embodiment of the invention drawn in FIG. 29W, wherein the ends of the arms of the flexible longitudinal fixation component 3202 were welded together. FIG. 32B is a lateral view of a partial sagittal cross section of the spinal segment and the embodiment of the invention drawn in FIG. 32A. A threaded vertebral fixation component 3208 was screwed into the vertebra followed by passing one arm of the flexible longitudinal fixation component through the intra-aperture component 3210 then through the AF tissue cranial to the aperture. The invention taught in FIGS. 37A-F could be used to pass the end of the flexible longitudinal fixation component through the AF tissue. The device could be manufactured with the materials listed in the text of FIG. 29C-W and the components could be similar in size to the size of the components listed in FIGS. 29C-W.

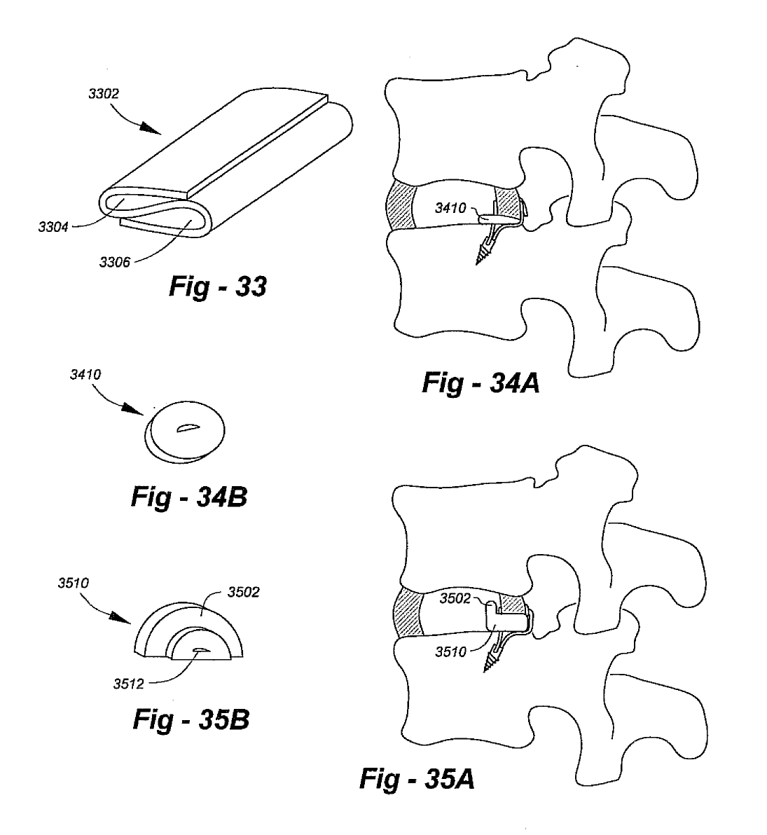

FIG. 33 is an oblique view of an alternative embodiment of an intra-aperture component. The component 3302 was manufactured by folding allograft tissue, synthetic mesh, or other material and stitching or otherwise fastening the layers of material together in a manner that creates one or more transverse passageways 3204, 3206. Other shapes or folding arrangements could be used in alternative embodiments of the invention.

FIG. 34A is a lateral view of a partial sagittal cross section of a spinal segment and an alternative embodiment of the invention wherein the intra-aperture component 3410 does not extend completely through the aperture and does not contain transverse passageways, and may not contain pores. The intra-aperture component could be made of metal such as titanium, plastic, polyethylene or other similar material. The device could be manufactured with the materials listed in the text of FIG. 29C-W and the components could be similar in size to the size of the components listed in FIGS. 29C-W. FIG. 34B is an oblique view of the embodiment of the intra-aperture component 3410 drawn in FIG. 34B.

FIG. 35A is a lateral view of a partial sagittal cross section of a spinal segment and an alternative embodiment of the invention wherein the distal end 3502 of the intra-aperture component 3510 is enlarged. A transverse passageway is evident at 3512. The device could be manufactured with the materials listed in the text of FIG. 29C-29W and the components could be similar in size to the size of the components listed in FIGS. 29C-29W.

FIG. 35B is an oblique view of the embodiment of the intra-aperture component 3510. The device could be made of composite materials. For example, the enlarged distal end of the device could be made of expanded polytetrafluoroethylene (ePTFE), polyester, polypropylene, fascia, dermis, or other synthetic material or tissue and the smaller portion of the device be made of allograft AF or other synthetic material or tissue. The components could be fastened together by a welded suture loop that passes through both components. The components could be connected with alternative methods in other embodiments of the invention.

FIG. 36A is a transverse cross section of an IVD and an instrument according to the invention that can be used to safely pass the arms of flexible longitudinal fixation components, of the embodiments of the invention drawn in FIGS. 29C-48E, through the AF 3602. The distal end of the device, foot plate 3604, was placed through an aperture in the AF and rests against the inner portion of the AF. The foot plate is preferably 3 to 8 millimeters in length, 2 to 4 millimeters wide and 1 to 3 millimeters tall. Alternative sizes of the foot plate could be used in alternative embodiments of the invention.

FIG. 36B is a transverse cross section of the IVD, the embodiment of the invention drawn in FIG. 36B, and the second step to pass an arm of the flexible longitudinal fixation component through the AF. The cannula 3610 and taper-tipped stylet 3612 were advanced through the AF followed by partial withdraw of the stylet. The stylet is preferably 0.5 to 2.0 millimeters in diameter. The cannula is preferably 1 to 3 millimeters larger in diameter than the stylet. The assembled tool is preferably 15 to 40 millimeters long or longer.

FIG. 36C is a transverse cross section of the IVD, the embodiment of the invention drawn in FIG. 36B, and the third step to pass an arm of a flexible longitudinal component through the AF. The arm of the flexible longitudinal fixation component was inserted into the cannula 3610, after removing the stylet. The distal portion of the arm of the flexible longitudinal fixation component could be coated with plastic or other material to stiffen the component. Stiffening the component facilitates advancement of the component through the cannula. The handle 3620 of the instrument was compressed to advance a vertical component that slides along the shaft of the instrument into a transverse component that slides along the foot-plate of the distal end of the tool. The transverse sliding component 3622 presses against the side of the portion of the flexible longitudinal fixation component that was passed through the AF and the foot-plate of the tool. The feature 3630 grasps the distal end of the flexible longitudinal fixation component 3632.

FIG. 36D is a lateral view of a sagittal cross section of the distal portion of the instrument drawn in FIG. 36C. The instrument was drawn in its flexible longitudinal fixation component-grasping position.

FIG. 36E is an exploded transverse cross section of the IVD, the embodiment of the invention drawn in FIG. 36C, and the fourth step in the method of passing an arm of the flexible longitudinal fixation component through the AF. The cannula 3610 was removed from the insertion instrument. The flexible longitudinal fixation component 3632 was passed through openings 3634, 3636 in the side of the tool.

FIG. 36F is a view of the top of the insertion tool drawn in FIG. 36E. Similar to the invention drawn in FIG. 29S, the opening in the side of the tool captures the cannula but allows the flexible longitudinal fixation component to escape the tool once the cannula is removed from the tool.

FIG. 36G is a transverse cross section of the IVD, the embodiment of the invention drawn in FIG. 36E, and the final step to pass the arm of a flexible longitudinal fixation component 3632 through the AF. The distal end of the flexible longitudinal fixation component is pulled through the aperture of the AF as the tool is extracted from the IVD. A second flexible longitudinal fixation component could be passed through the AF tissue on the opposite side of the aperture, the proximal ends of the flexible longitudinal fixation components could be welded together and welded area of the components pushed through the aperture. A sleeve or sleeves could be placed over the welded area of the flexible longitudinal fixation components to help protect the weld from forces that peel the weld apart. Tension could be applied to the distal ends of the flexible longitudinal fixation components closing the aperture followed by welding the distal ends of the components to each other. The tool could be used to pass a wire loop through the AF rather than a flexible longitudinal fixation component, similar to the method taught in FIGS. 37A-F, in alternative embodiments of the invention.

FIG. 37A is a transverse cross section of the IVD, a flexible longitudinal fixation component 3632 that was passed through the AF 3602 using the embodiment of the invention drawn in FIGS. 36A-G and an alternative embodiment of the invention drawn in FIGS. 36A-G. The distal end of a wire-passing tool 3702 was inserted through the aperture 3704 in the AF and rests against the inner portion of the AF 3602. The dimensions of the tool are similar to the dimensions of the tool drawn in FIG. 36A.

FIG. 37B is a transverse cross section of the IVD, the embodiment of the invention drawn in FIG. 37A, and the second step in the method of passing a flexible longitudinal fixation component through the AF. The handle of the tool was compressed to drive the sharp distal tip 3706 of the tool through the AF and into an opening on a second foot-like component 3708 of the tool. The second foot-like component that rests against the outer portion of the AF provides counter pressure on the AF while the tip of the tool is forced through the AF and shields the nerves within the spinal canal from the sharp tip of the instrument. The distal end of a wire loop 3710 was passed through the cannulated shaft of the instrument, through the AF, and through an opening in the outer foot-plate portion of the tool.

FIG. 37C is a transverse cross section of the IVD, the embodiment of the invention drawn in FIG. 37B, and the third step in the method of passing a flexible longitudinal fixation component through the AF. The distal end of the wire loop 3710 was captured by a hook shaped instrument 3720, the wire passing tool was removed from the IVD, and the wire loop was pulled through a slot-like opening in the side of the outer foot-plate 3708 of the tool 3702.

FIG. 37D is a transverse cross section of the IVD, the embodiment of the invention drawn in FIG. 37C, and the fourth step in the method of passing a flexible longitudinal fixation component through the AF. The distal end of the previously passed flexible longitudinal fixation component 3632 was passed through the opening in the proximal end of the wire loop 3710.

FIG. 37E is a transverse cross section of the IVD, the embodiment of the invention drawn in FIG. 37D, and the fifth step in the method of passing a flexible longitudinal fixation component through the A-F Tension on the proximal end of the wire loop 3710 pulls the distal end of the wire loop and the distal end of the flexible longitudinal fixation component 3632 through the aperture 3704 in the AF 3602. The distal end of the flexible longitudinal fixation component 3632 could be welded to the side of the component after passing the distal end of the component through the wire loop 3710 to prevent premature dissociation of the component from the wire loop. The distal end of the flexible longitudinal fixation component 3632 could be passed through devices such as a sheet of mesh before it is passed through AF tissue a second time. Such invention fastens the device to the inner portion of the AF as taught in FIG. 10G.

FIG. 37F is an exploded transverse cross section of the IVD, the embodiment of the invention drawn in FIG. 37E, and the sixth step in the method of passing a flexible longitudinal fixation component through the AF. The ends of the component 3632 were passed through the AF 3602 on either side of the aperture 3704.

FIG. 38A is an oblique view of a tube 3802 used to create an alternative embodiment of the invention drawn in FIG. 26A. The device is preferable made of expanded polyfluoroethylene (ePTFE). Alternatively, the device could be made of other biocompatible materials. FIG. 38B is a posterior view of the tube 3802. The dotted lines indicate areas to cut the posterior wall of the tube. Circular or other shaped openings 3804 were created in the central portion of the posterior side of the wall. Such openings are preferably 0.1 to 2.0 mm in diameter. The device is preferably 8 to 45 millimeters long, 4 to 20 millimeters wide and 0.6 to 2.0 millimeters thick.

FIG. 38C is a posterior view of the embodiment of the invention drawn in FIG. 38B. The flaps 3810, 3812 cut into the posterior wall of the ends of the tube were unfolded as shown in the drawing. FIG. 38D is an anterior view of the embodiment of the invention drawn in FIG. 38C.

FIG. 38E is an anterior view of the embodiment of the invention drawn in FIG. 3D. A slit 3820 was cut through the anterior wall of the device. The tip of a welding instrument can be placed through such opening to weld the ends of sutures that were passed through the lumen of the device. One or more slits may be created in the side walls of the device in alternative embodiments of the invention. The slits are preferably 3 to 15 millimeters long.

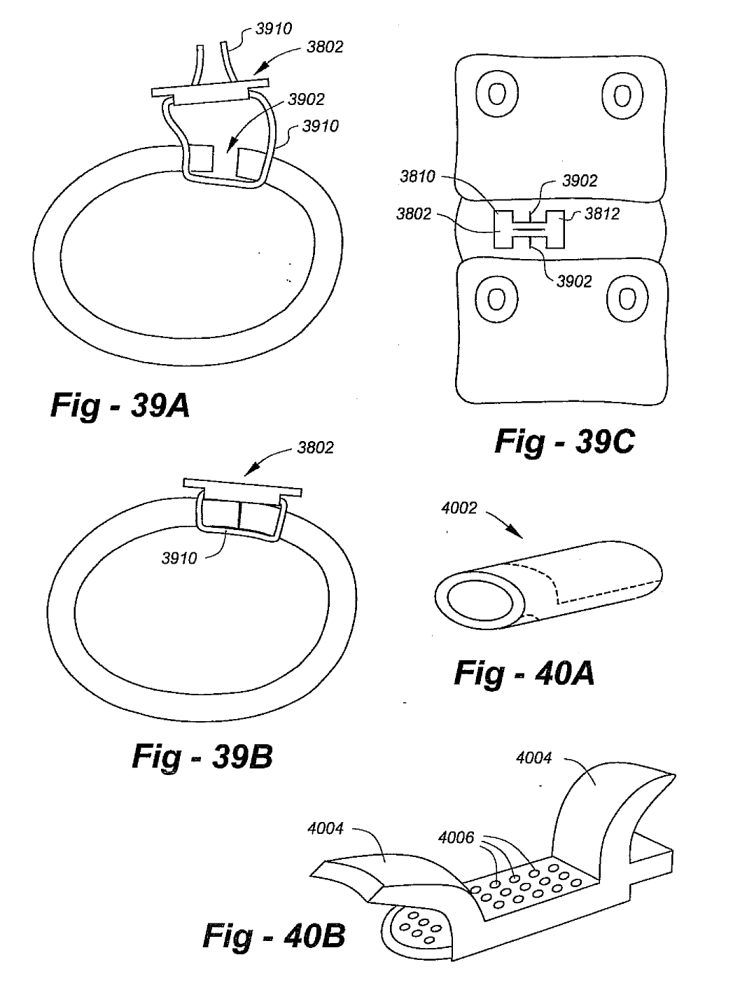

FIG. 39A is a transverse cross section of the IVD drawn in FIG. 37F and the embodiment of the invention drawn in FIG. 38E. The ends of the flexible longitudinal fixation component 3910 were passed through the lumen and the anterior opening of the device 3802.

FIG. 39B is a transverse cross section of the IVD and the embodiment of the invention drawn in FIG. 39A. The aperture 3902 was closed by applying tension on the ends of the flexible longitudinal fixation component 3910 and followed by welding the ends of the flexible longitudinal fixation component. The welded area of the flexible longitudinal component lies in the embodiment of the invention drawn in FIG. 38E. FIG. 39C is a posterior view of a coronal cross section of a spinal segment and the embodiment of the invention drawn in FIG. 39B.

FIG. 40A is an oblique view of an alternative tube 4002 according to the invention. The dotted lines indicate places to cut the posterior and side walls of the device. The device is preferably manufactured with the material listed in the text of FIG. 38A. The dimensions of the device are similar to the dimensions of the embodiment of the invention listed in the text of FIG. 38A.

FIG. 40B is an oblique view of the embodiment of the invention drawn in FIG. 40A. A flap 4004 of the anterior wall of the device was raised to expose the holes 4006 in the posterior wall of the device 4002. Raising the door-like flap facilitates welding the ends of the flexible longitudinal fixation component. The flap is preferably 3 to 15 millimeters long.

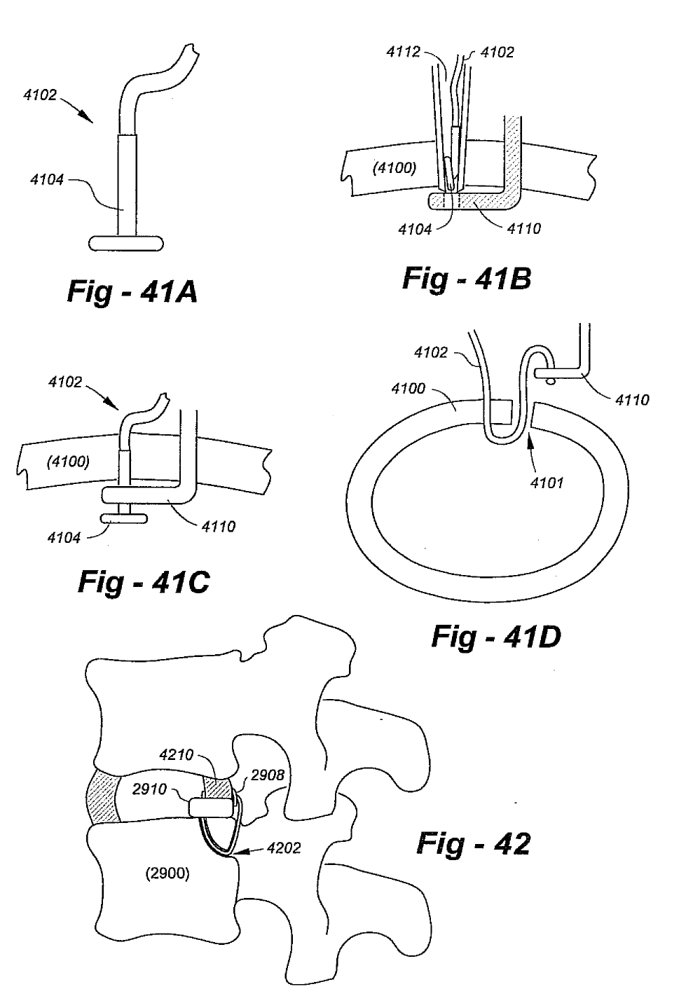

FIG. 41A is a lateral view of the distal end of a flexible longitudinal fixation component 4102. The T-shaped end 4104 of the component is made of plastic. FIG. 41B is a view of a partial transverse cross section of a portion of an IVD, the foot-plate 4110 of an insertion tool, a cannula 4112 and the end of the flexible longitudinal fixation component drawn in FIG. 41A. The T-shaped end was folded and forced through a cannula that was passed through the AF 4100. The dimensions of the tool are similar to the dimensions of the tool drawn in FIG. 36A.

FIG. 41C is view of a partial transverse cross section of the portion of the IVD and embodiment of the invention drawn in FIG. 41B. The folded T-shaped component returned to the T-shape after it was passed through and opening in the foot-plate of the tool. The feature increases the force required to pull the end of the flexible longitudinal fixation component from the tool.

FIG. 41D is a view of transverse cross section of the IVD and embodiment of the invention drawn in FIG. 41C. The end of the flexible longitudinal fixation component 4102 and foot-plate 4110 of the tool were pulled through the aperture 4101 in the AF 4100. The T-shaped end of the flexible longitudinal fixation component 4102 is cut and removed after passing the component through the AF 4100.

FIG. 42 is a lateral view of a partial sagittal cross section of a spinal segment and an alternative embodiment of the invention drawn in FIG. 29W. The inferior arm of the flexible longitudinal component was passed through a hole 4202 in the vertebra 2900 and welded to the superior arm of the flexible longitudinal fixation component after passing the superior arm of the flexible longitudinal component through the AF. The device could also be manufactured with the materials listed in the text of FIG. 29C-W. The embodiment of the invention is also similar in size to the embodiment of the invention drawn in FIGS. 29C-W.

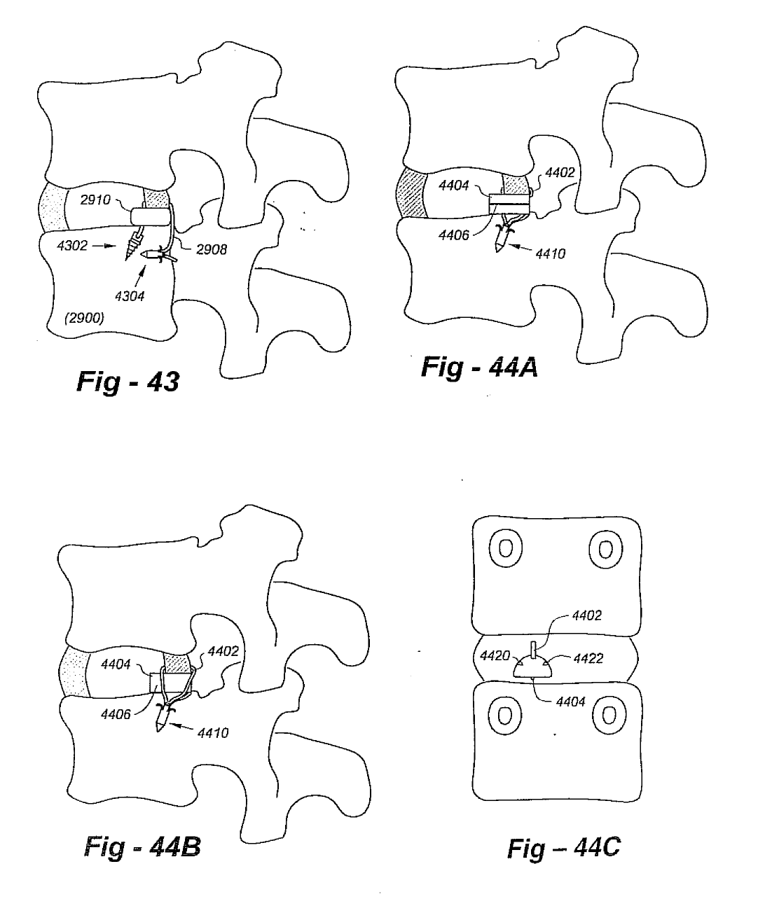

FIG. 43 is a lateral view of a partial sagittal cross section of a spinal segment and an alternative embodiment wherein the flexible longitudinal fixation component 2908 was passed through a threaded vertebral fixation component 4302 that was screwed through the VEP and into the vertebra 2900. Both arms of the flexible longitudinal fixation component were passed through the intra-aperture component 2910, through the AF 4210, and through a locking mechanism 4304 in a vertebral fixation component that was impacted into the posterior portion of vertebral body 2900. The device could similar to the size of the embodiment of the invention drawn in FIGS. 29C-W and be manufactured with the materials listed in the text of FIG. 29C-W.

FIG. 44A is a lateral view of a partial sagittal cross section of a spinal segment and an alternative embodiment of the invention drawn in FIG. 29V. The arms of the flexible longitudinal fixation component 4402 passes through two converging vertical passageways in the inter-aperture component 4404. Grooves 4406 along the sides of the inter-aperture component allow escape of NP tissue. The grooves are preferably 1.1 to 3 millimeters deep. Alternatively, the grooves may be deeper than 3 millimeters. As a further alternative, one or more passageways may be used instead of or in combination with the groove(s) 4406. This embodiment of the invention and other embodiments of the invention taught in FIGS. 29C-48E could be rotated 180 degrees to treat apertures of the AF near The vertebra cranial to the IVD. The device could similar to the size of the embodiment of the invention drawn in FIGS. 29C-W and be manufactured with the materials listed in the text of FIG. 29C-W.

FIG. 44B is a lateral view of a partial sagittal cross section of a spinal segment and embodiment of the invention drawn in FIG. 44A. The guide taught in FIG. 29R enables precise placement of the vertebral fixation component 4410 relative to portions of the flexible longitudinal fixation element 4402 that extend from the intra-aperture component 4404. Vertebral fixation components placed too posterior through the VEP relative to the location of the portions of the flexible longitudinal fixation elements that extend from the intra-aperture component 4404, could cause the intra-aperture component 4404 to project into the spinal canal, thus compressing the nerves.

FIG. 44C is a posterior view of a coronal cross section of the spinal segment and embodiment of the invention drawn in FIG. 44B. The superior arm of the flexible longitudinal fixation component 4402 preferably passes through the AF tissue within 1 to 3 millimeters of the VEP. The AF tissue in such location, is less damaged and stronger than the AF tissue within 1 to 3 millimeters of the VEP. The invention drawn in FIG. 26B could be applied over the portion of the flexible longitudinal fixation component that sits outside the AF, in this and other embodiments of the invention taught FIGS. 29C-48E in this application.

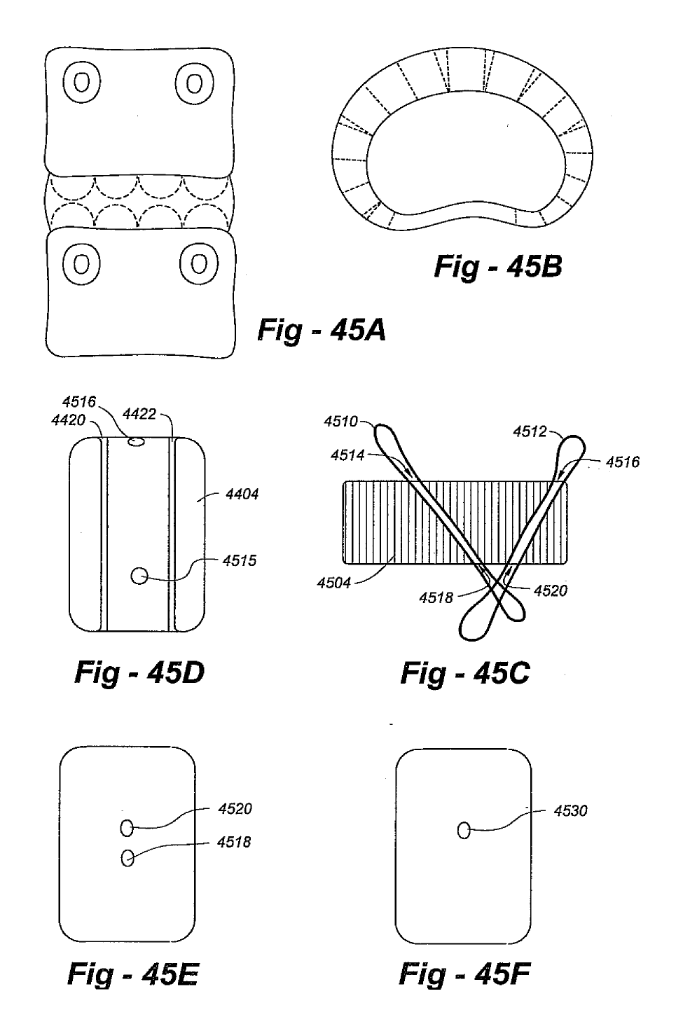

FIG. 45A is an anterior view of an allograft or xenograft spinal segment. The dotted lines indicate where to cut the IVD to manufacture the inter-aperture components drawn in FIG. 29C-48E. FIG. 45B is transverse cross section of the IVD drawn in FIG. 45A. The dotted lines indicate where to cut the IVD to manufacture the inter-aperture component drawn in FIGS. 29C-48E.

FIG. 45C is a lateral view of a sagittal cross section of the inter-aperture invention drawn in FIG. 44C. Wire loops 4510, 4512 were placed into the converging vertical passageways in the component 4404 to facilitate passage of the ends of the flexible longitudinal fixation element 4402 through the intra-aperture component 4404. The oblique course of the passageways enables the ends of flexible longitudinal fixation element to pass through multiple lamellae of the AF. The vertical lines in the drawing represent the lamellae. Our previously described suture pullout study indicates sutures that pass through multiple lamellae have a high resistance to pullout (average 36N/mm of AF tissue). The lamellae of the graft are aligned with the native lamellae of the damaged disc to maximize the strength of the healed IVD. Each lamellae of the healed allograft component provides maximal resistance to NP tissue extrusion. However, as previously noted the transverse passageways, grooves in this embodiment of the invention, enable NP particle extrusion until the connective tissue grows into the graft.

FIG. 45D is view of the top of the embodiment of the intra-aperture component drawn in FIG. 44C. The drawing shows grooves 4420, 4422 along the sides of the device 4404. The entrance 4516 to the vertical passageway closest to the proximal end of the component preferably enters the proximal end of the device or within 1 to 2 millimeters of the proximal end of the component.

FIG. 45E is a view of the bottom of the embodiment of the invention drawn in FIG. 45D. The hole 4520 closest to the proximal end of the component preferably exits the bottom of the component within 2 to 8 millimeters of the proximal end of the component. Alternatively, the hole may exit within 1 millimeter or less of the proximal end of the component or more than 8 millimeters from the proximal end of the component. FIG. 45F is a view of the bottom of an intra-aperture component wherein the vertical passageways exit through a single hole 4530 in the bottom of the component.

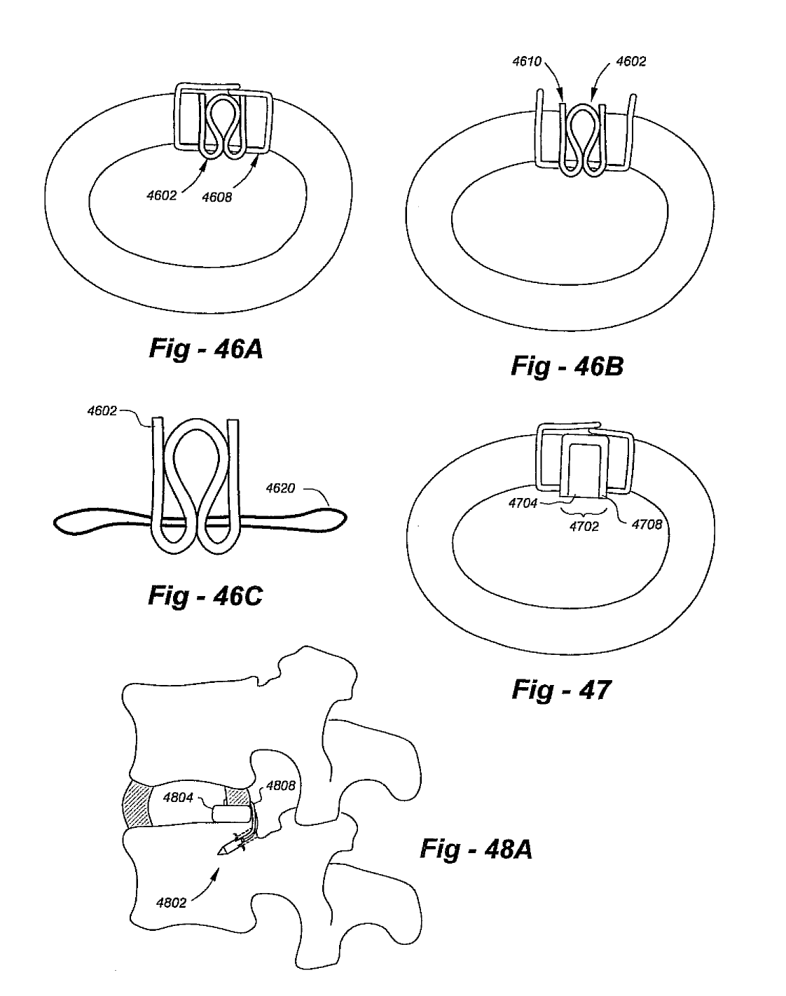

FIG. 46A is a transverse cross section of an IVD and an alternative embodiment of the invention drawn in FIG. 30B A piece 4602 of allograft or xenograft fascia, dermis, or other tissue, or a piece of synthetic material such as polyester mesh was folded and fastened to the AF using the method taught in FIG. 30B. The distal end of the flexible longitudinal fixation component 4608 was passed through holes near the distal end of the folds of the intra-aperture component 4602. The fold or folds at the proximal end of the component have passageways through the fold to enable the escape of NP tissue. The device could similar to the size of the embodiment of the invention drawn in FIGS. 29C-W and be manufactured with the materials listed in the text of FIG. 29C-W.

In all embodiments of the invention utilizing an intra-aperture component, the proximal surface of the intra-aperture component is preferably flush with or recessed by a few millimeters relative to the outer surface of the AF to prevent pressure applied to the spinal column. FIG. 46B is a view of a transverse cross section of the IVD and the embodiment of the invention drawn in FIG. 46A. The drawing demonstrates the importance of the length of the intra-aperture component, the position of the holes for the flexible longitudinal fixation component 4608 and the relationship of such dimensions to the width of the AF tissue surrounding the aperture. The intra-aperture component in the drawing is too long or the holes are positioned too near the distal end of the component. The suboptimal dimensions of the intra-aperture component cause it to project beyond the surface of the IVD at 4610, which may lead to nerve compression.

FIG. 46C is a view of the top of the embodiment of the intra-aperture component drawn in FIG. 46A. A wire loop 4620 was inserted through the holes of the device to facilitate passage of an end of the flexible longitudinal fixation component through the intra-aperture component. The intra-aperture component is preferably supplied to surgeons in packaging the notes the width, length, height of the component and the distance from the transverse passageway to the proximal end of the component. Surgeons preferably measure the size of the aperture in the AF with a sizing tool such as drawn in FIG. 29F and the thickness of the AF with calipers to avoid inserting intra-aperture components that extend into the spinal canal.

The intra-aperture component is preferably provided to surgeons in a variety of sizes including devices: 1) with distances of 2 to 8 millimeters between the transverse passageway and the proximal end of the device, 2) heights of 3 to 8 millimeters, 3) widths of 3 to 8 millimeters and 4) lengths of 3 to 9 millimeters. Larger or smaller components may be used in other embodiments of the invention. The embodiments of the invention taught in FIGS. 29C-35B, 39A-C, 42-44C, & 46A-48E could be supplied hilly assembled I various sizes or shapes or supplied as separate components of various sizes and shapes to enable surgeons to customize the assembled device to each patient.

FIG. 47 is a transverse cross section of an IVD and an alternative embodiment of the invention wherein a composite intra-aperture component 4702 was fastened to the AF. For example, a piece of allograft or xenograft AF or other tissue 4704 may be covered with a sleeve or sling 4708 made of an alternative material such as fascia, dermis, polyester, nylon, or polypropylene mesh. The sleeve could increase the tensile strength of the composite component to help prevent the flexible longitudinal fixation component from tearing through the intra-aperture component. The device could similar to the size of the embodiment of the invention drawn in FIGS. 29C-W and be manufactured with the materials listed in the text of FIG. 29C-W.

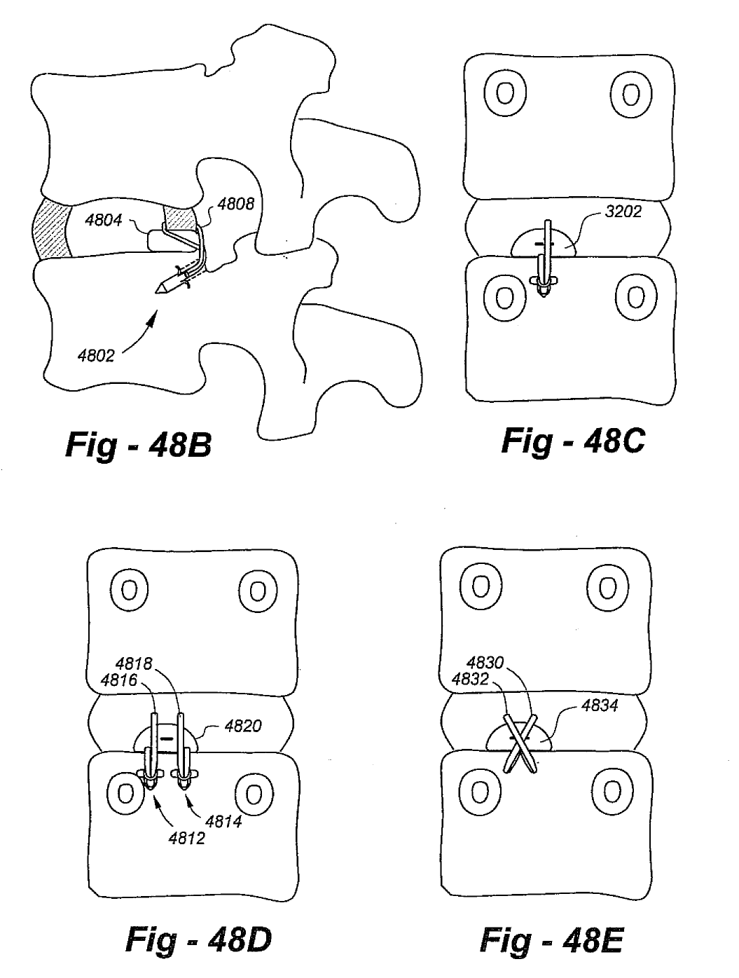

FIG. 48A is a lateral view of a partial sagittal cross section of a spinal segment and an alternative embodiment of the invention drawn in FIG. 44A. The vertebral fixation component 4802 was inserted into the posterior portion of the vertebral body. Such component is preferably inserted into the vertebra within 1 to 6 millimeters of the VEP. Alternatively, the anchor may be inserted 7, 8, 9, 10, or more millimeters of the VEP or placed through the junction of the VEP and the posterior vertebral body in alternative embodiments of the invention. The vertebral fixation component is preferably recessed 3 to 15 millimeters anterior to the posterior surface of the vertebral body. The device could similar to the size of the embodiment of the invention drawn in FIGS. 29C-W and be manufactured with the materials listed in the text of FIG. 29C-W.

FIG. 48B is a lateral view of a partial sagittal cross section of the spinal segment and the embodiment of the invention drawn in FIG. 48A. The flexible longitudinal fixation component 4808 was passed through a diagonal passageway through the intra-aperture component 4804. The diagonal passageway preferably exits the proximal end of the intra-aperture component within 1 to 3 millimeters of the bottom component. Alternatively, the diagonal passageway could preferably exit through the bottom of the intra-aperture component within 1 to 3 millimeters of the proximal end of the component. Alternatively, the diagonal passageway may exit at the junction of the proximal end and bottom of the intra-aperture component or within 4, 5, 6, millimeters of such area. The superior arm of the flexible longitudinal component preferably passes over the proximal end of the intra-aperture component but may pass through the proximal 1 to 3 millimeters of the intra-aperture component.

FIG. 48C is a posterior view of a coronal cross section of the spinal segment and the embodiment of the invention drawn in FIG. 48B. The superior arm of the flexible longitudinal fixation component could be covered with a sleeve similar to the embodiment of the invention drawn in FIG. 38D.

FIG. 48D is a posterior view of a coronal cross section of a spinal segment and an alternative embodiment of the invention drawn in FIG. 48C. The device has two vertebral fixation components 4812, 4814, two flexible longitudinal fixation components 4816, 4818, and one intra-aperture component 4820. Three four or more vertebral fixation components, three or more flexible longitudinal fixation components, and two or more intra-aperture components could be used in alternative embodiments of the invention. The vertebral fixation components could be made of resorbable materials such as polylactic acid (PLA) and/or polyglycolic acid (PGA) in this and other embodiments of the invention taught in this application. The device could similar to the size of the embodiment of the invention drawn in FIGS. 29C-W and be manufactured with the materials listed in the text of FIG. 29C-W.

FIG. 48E is a posterior view of a coronal cross section of a spinal segment and an alternative configuration wherein the flexible longitudinal fixation components 4830, 4832 cross one another over the proximal end of the intra-aperture component 4834 and may cross one another within the intra-aperture component in this embodiment of the invention. The device could similar to the size of the embodiment of the invention drawn in FIGS. 29C-29W and be manufactured with the materials listed in the text of FIG. 48D.

1. Apparatus for occluding a defect in the anulus fibrosis (AF) of an intervertebral disc (IVD) between upper and lower vertebral bodies, the AF having an inner surface and an outer surface, the inner surface of the AF defining an intervertebral space including nucleus pulposus (NP) tissue, the apparatus comprising:
an intra-aperture component dimensioned for positioning within the defect, the intra-aperture component having a length, an outer wall between a proximal surface and a distal surface, and a cross-section with vertical and horizontal orientations;
one or more components for maintaining the intra-aperture component in position within the defect; and
one or more lengthwise passageways through the intra-aperture component, one or more lengthwise grooves on the outer surface of the intra-aperture component, or a combination thereof to intentionally facilitate the escape of nucleus pulposus tissue through or around the intra-aperture component in response to pressure applied by the upper and lower vertebral bodies.

2. The apparatus of claim 1, wherein the intra-aperture component is porous

3. The apparatus of claim 1, wherein the intra-aperture component is flexible.

4. The apparatus of claim 1, wherein the intra-aperture component is intentionally non-expandable at least in cross section following its positioning within the defect

5. The apparatus of claim 1, wherein the components used to maintain the intra-aperture component within the defect includes a flexible longitudinal fixation component that passes through the intra-aperture component and a region of the AF apart from the defect.

6. The apparatus of claim 1, wherein the components used to maintain the intra-aperture component within the defect includes a flexible longitudinal fixation component that passes through a generally vertical passageway in the intra-aperture component and a region of the AF apart from the defect.

7. The apparatus of claim 1, wherein the components used to maintain the intra-aperture component within the defect includes a flexible longitudinal fixation component that passes through a generally vertical passageway in the intra-aperture component and a region of the AF having overlapping layers with intact fibers in different directions.

8. The apparatus of claim 1, wherein:
the components used to maintain the intra-aperture component within the defect includes a flexible longitudinal fixation component that passes through a generally vertical passageway in the intra-aperture component and a region of the AF apart from the defect; and
the vertical passageway does not intersect with any lengthwise passageway.

9. The apparatus of claim 1, wherein:
the components used to maintain the intra-aperture component within the defect includes a flexible longitudinal fixation component that passes through the intra-aperture component; and
the flexible longitudinal fixation component is anchored to one of the upper and lower vertebral bodies.

10. The apparatus of claim 1, wherein:
the components used to maintain the intra-aperture component within the defect includes a flexible longitudinal fixation component that passes through the intra-aperture component; and
the flexible longitudinal fixation component is anchored to one of the upper and lower vertebral bodies with an anchor with arms that expand following implantation.

11. The apparatus of claim 1, wherein the components used to maintain the intra-aperture component within the defect includes a flexible longitudinal fixation component that passes twice through the intra-aperture component and is anchored to one of the upper and lower vertebral bodies.

12. The apparatus of claim 1, wherein the components used to maintain the intra-aperture component within the defect includes a flexible longitudinal fixation component anchored to one of the upper and lower vertebral bodies, flexible longitudinal fixation component forming one or more loop or loops, each passing once through the AF and twice through the intra-aperture component.

13. The apparatus of claim 1, wherein the proximal surface of the intra-aperture component is flush with or recessed relative to the outer surface of the AF.

14. The apparatus of claim 1, wherein the flexible longitudinal fixation component is composed of suture material.
